(19) United States
(12) Patent Application Publication
(10) Pub. No.: US 2024/0379207 A1
(43) Pub. Date: Nov. 14, 2024

(54) WEARABLE ELECTRONIC DEVICE FOR COOPERATION WITH OTHER DEVICE FOR MANAGING HEALTH, AND OPERATION METHOD OF THE SAME

(71) Applicant: INHANDPLUS INC., Pohang-si, Gyeongsangbuk-do (KR)

(72) Inventor: Hwiwon LEE, Seoul (KR)

(73) Assignee: INHANDPLUS INC., Pohang-si, Gyeongsangbuk-do (KR)

(21) Appl. No.: 18/586,665

(22) Filed: Feb. 26, 2024

(30) Foreign Application Priority Data
May 12, 2023 (KR) 10-2023-0061787
Jun. 21, 2023 (KR) 10-2023-0079486
Jul. 11, 2023 (KR) 10-2023-0089855
Jul. 21, 2023 (KR) 10-2023-0095144
Jan. 10, 2024 (KR) 10-2024-0003964
Jan. 10, 2024 (KR) 10-2024-0003965
Jan. 10, 2024 (KR) 10-2024-0003966
Jan. 10, 2024 (KR) 10-2024-0003967
Jan. 10, 2024 (KR) 10-2024-0003968
Jan. 10, 2024 (KR) 10-2024-0003969

(51) Int. Cl. G16H 20/10; G16H 40/67

(52) U.S. Cl. CPC G16H 20/10; G16H 40/67

(57) ABSTRACT

According to various embodiments, a device is provided for medication determination, including: a sub device including a first fastening structure configured to be detachable from a main body, the sub device being configured to be operated independently from the main body, wherein the sub device includes: at least one processor; a short range communication circuit; and a camera disposed to an external surface of the sub device; wherein, while the sub device is coupled to the main body, the at least one processor is configured to: establish, through the short range communication circuit, a communication connection with the main body, capture an image related to a medication performed by a user using the camera, and provide the image cause the main body to obtain a result information of the medication determination.

### CROSS-REFERENCE TO RELATED APPLICATIONS

The present application claims priority to Korean Patent Applications No. 10-2023-0061787, filed May 12, 2023, No. 10-2023-0079486, filed Jun. 21, 2023, No. 10-2023-0089855, filed Jul. 11, 2023, No. 10-2023-0095144, filed Jul. 21, 2023, No. 10-2024-0003964, filed Jan. 10, 2024, No. 10-2024-0003965, filed Jan. 10, 2024, No. 10-2024-0003966, filed Jan. 10, 2024, No. 10-2024-0003967, filed Jan. 10, 2024, No. 10-2024-0003968, filed Jan. 10, 2024 and No. 10-2024-0003969, filed Jan. 10, 2024, the entire contents of which are incorporated herein for all purposes by this reference.

### BACKGROUND

### 1. Field

The present disclosure relates to a wearable electronic device for collaboration with another device for managing health, and an operation method of the same.

### 2. Description of Related Art

Health management based on medical and medical studies has been steadily attracted since now, and as expected lifespan has increased due to recent advances in medical technology, their attention has increased. Such health management should be regularly performed by the user by itself in daily life, such as taking drugs prescribed in daily life, periodically injecting drugs into the body, or measuring health indicators using medical devices.

In order to improve the above-described effects of health management, it is necessary to induce or impose forcibilities of users related to health management, and for this, a monitoring system is required to check whether health management is well performed by detecting users related to health management behaviors in daily life.

Meanwhile, in the conventional monitoring of the user's health care-related behavior, the user's behavior was required, such as driving the monitoring device before the drug implementation or recording their own behavior by itself, so that there was a problem in that when the user dismissed their own behavior, the information about the health care was not collected.

Therefore, in consideration of the user's convenience and the accuracy of the health care, there is a need to develop a system that automatically monitors the health care-related behavior even without the user's behavior in the daily life. Meanwhile, as a patent for sensing a medication status by using a camera, there is a Korean Patent Publication No. 2019-0078752, “Intelligent Information Management System for Storage and Medication Management for Smart House Drugs”, published date 2019.07.05.

### SUMMARY

An objective of the present invention is to provide a monitoring system that automatically monitors behaviors related to health management of users in daily life.

An objective of the present invention is to provide a monitoring system that senses the movement of the user when the user medicates, captures the medication image of the user, analyzes the captured image, and determines whether the medication is performed.

An objective of the present invention is to provide a wireless communication device that senses a user's behavior and generates a photographing start signal for photographing a user's medicine performance-related image.

An objective of the present invention is to provide a wearable device that can be worn on at least a part of a user's body and, when receiving the photographing start signal, opens a closed camera to photograph a user's medicine performance-related image.

An objective of the present invention is to provide a server that analyzes a user's medicine dose-related image using an artificial neural network to determine whether the user correctly takes the medicine.

An objective of the present invention is to provide a sub-wearable device that can support a health management service in collaboration with a wearable device manufactured by a manufacturer of a third party.

An objective of the present invention is to provide a server that enhances the quality of the user's life log by using a correlation between the medicine dose and the life log obtained by using a sensor.

An objective of the present invention is to provide a server that enables adjustment of the amount of desire for disease improvement by using a correlation between the medicine dose and the value of disease symptoms obtained by using a sensor.

An objective of the present invention is to provide a server that determines whether the user correctly takes the medicine based on the value sensed by various sensors as well as the image.

The objective of the present invention is not limited to the above technical problems, and the technical problems not mentioned may be clearly understood by those skilled in the art from the specification and the accompanying drawings.

According to various embodiments, a device may be provided for medication determination, including: a sub device including a first fastening structure configured to be detachable from a main body, the sub device being configured to be operated independently from the main body, wherein the sub device includes: at least one processor; a short range communication circuit; and a camera disposed to an external surface of the sub device; wherein, while the sub device is coupled to the main body, the at least one processor is configured to: establish, through the short range communication circuit, a communication connection with the main body, capture an image related to a medication performed by a user using the camera, and provide the image cause the main body to obtain a result information of the medication determination.

According to various embodiments, an operation method of device may be provided for medication determination including a main device and a sub device configured to be detachable from the main body, comprising: establishing, through the short range communication circuit, a communication connection with the main body; capturing an image related to a medication performed by a user using the camera, and providing the image cause the main body to obtain a result information of the medication determination.

According to various embodiments, the technical solutions are not limited to the above-described solutions, and the unmentioned solutions can be clearly understood by those skilled in the art from the present specification and the accompanying drawings.

According to an example of the present specification, the user's convenience can be improved by automatically monitoring the health management of the user and minimizing the user's intervention.

According to an example of the present specification, a wearable device worn on the user and photographing an image related to the medication of the user can be provided to casily monitor the health management of the user in everyday life, thereby increasing usability.

According to an example of the present specification, a wireless communication device that senses the operation of the user and generates a signal for photographing the image related to the medication can be provided to more accurately and regularly monitor whether the user has a medication.

According to an example of the present specification, the accuracy of the determination of whether the medication can be improved by analyzing the image related to the medication using an artificial neural network.

According to an example of the present specification, a sub-wearable device that can support a health management service in collaboration with a wearable device manufactured by a manufacturer of a third party can be provided to improve the convenience for the health management of the user.

According to an example of the present specification, a server that improves the quality of the user's life log using a correlation between the amount of drug consumption and the life log obtained using the sensor can be provided to accurately perform the medications for health improvement.

According to an example of the present specification, a server that uses a correlation between the amount of drug consumption and the value of disease symptoms obtained using the sensor can be provided to adjust the amount of desire for disease improvement.

According to an example of the present specification, a server that determines whether the user has correctly taken the drug based not only on the image but also on the value sensed from various sensors can be provided to improve the convenience for the health management of the user.

The effects according to the present specification are not limited to the above-described effects, and the effects that are not mentioned can be clearly understood by those skilled in the art from the present specification and the accompanying drawings.

### BRIEF DESCRIPTION OF THE DRAWINGS

FIG. 1 is a block diagram for explaining an example of a configuration of a health management system according to various embodiments.

FIG. 2 is a block diagram for explaining another example of a configuration of a health management system according to various embodiments.

FIG. 3 is a diagram for explaining an example of a health management behavior according to various embodiments.

FIG. 4 is a diagram for explaining another example of a health management behavior according to various embodiments.

FIG. 5 is a block diagram showing an example of a component of a server according to various embodiments.

FIG. 6 is a diagram for explaining an example of at least one artificial intelligence model for obtaining health management data for a medication behavior according to various embodiments.

FIG. 7 is a diagram for explaining an example of a medication behavior according to various embodiments.

FIG. 8 is a diagram for explaining an example of an image obtained by photographing a medication behavior as training data for at least one artificial intelligence model according to various embodiments.

FIG. 9 is a block diagram showing an example of a component of a wearable device according to various embodiments.

FIG. 10 is a block diagram showing an example of a component of a user terminal according to various embodiments.

FIG. 11 is a block diagram showing an example of a component of a wireless communication device according to various embodiments.

FIG. 12 is a diagram illustrating an example of a wireless communication device attached to a target object according to various embodiments.

FIG. 13 is a flowchart illustrating an example of a basic operation for a medication action, which is an example of a health management behavior of a health management system according to various embodiments.

FIG. 14A is a perspective view of a wearable device according to some embodiments of the present disclosure.

FIG. 14B is a perspective view of a camera and a protective cover in a state in which a housing of a main strap is removed in some embodiments of the present disclosure.

FIG. 15A is a diagram illustrating an automatic shielding structure in some embodiments of the present disclosure.

FIG. 15B is a diagram illustrating an operation sequence of an automatic shielding protective cover and a camera in some embodiments of the present disclosure.

FIG. 16A is a diagram illustrating a structure of a manual shielding protective cover in some embodiments of the present disclosure.

FIG. 16B is a diagram illustrating an operation of a wearable device according to a closed state of a manual shielding protective cover in some embodiments of the present disclosure.

FIG. 17 is a diagram illustrating an operation of a wearable device according to an intermediate opening of a manual shielding protective cover in some embodiments of the present disclosure.

FIG. 18 is a diagram illustrating an AI learning method of a missing section according to an intermediate opening in some embodiments of the present disclosure.

FIG. 19A is a diagram illustrating an operation flow of a wearable device according to an intermediate opening of a manual shielding protective cover in some embodiments of the present disclosure.

FIG. 19B is a diagram illustrating an operation flow of a server according to an intermediate opening of a manual shielding protective cover in some embodiments of the present disclosure.

FIG. 20 is a block diagram illustrating an example of a health management system further including an add-on wearable device (e.g., a first wearable device) according to various embodiments.

FIG. 21 is a block diagram illustrating an example of a configuration of a first wearable device and a second wearable device according to various embodiments.

FIG. 22 is a diagram illustrating an example of a first wearable device and a second wearable device according to various embodiments.

FIG. 23 is a diagram illustrating an example of implementing the first wearable device in a single strap form according to various embodiments.

FIG. 24 is a diagram illustrating various examples of an add-on wearable device (e.g., the first wearable device) according to various embodiments.

FIG. 25 is a flowchart illustrating a basic operation of a health management system further including an add-on wearable device (e.g., the first wearable device) according to various embodiments.

FIG. 26 is a diagram illustrating an example of providing a health management service for taking a medication based on the first wearable device according to various embodiments.

FIG. 27 is a diagram illustrating an example of a communication connection method of the first wearable device according to various embodiments.

FIG. 28 is a flowchart illustrating an example of a communication connection setting operation of the first wearable device and the second wearable device according to various embodiments.

FIG. 29 is a flowchart illustrating a remote control operation of the first wearable device according to various embodiments.

FIG. 30 is a diagram illustrating an example of a remote control of the first wearable device according to various embodiments.

FIG. 31 is a flowchart illustrating an example of an operation for each of a plurality of modes of an add-on wearable device (e.g., the first wearable device) according to various embodiments.

FIG. 32 is a diagram illustrating an example of a plurality of modes of an add-on wearable device (e.g., the first wearable device) according to various embodiments.

FIG. 33 is a flowchart illustrating an example of an operation of another wearable device when a power of one of the first wearable device and the second wearable device is turned off according to various embodiments.

FIG. 34 is a flowchart illustrating an example of an operation of a health management system when the first wearable device is lost according to various embodiments.

FIG. 35 is a block diagram of a charging device according to various embodiments.

FIG. 36 is a diagram illustrating an example of a charging device according to various embodiments.

FIG. 37 is a flowchart illustrating operations of a charging device according to various embodiments.

FIG. 38 is a block diagram illustrating operations of performing medication determination further using at least one sensor of a wearable device, according to various embodiments.

FIG. 39 is a diagram illustrating activation signal reference conditions for each wearable sensor in some embodiments of the present disclosure.

FIG. 40 is a flowchart illustrating a process of generating an activation signal using a wearable sensor in some embodiments of the present disclosure.

FIG. 41 is a flowchart illustrating a process of generating an activation signal using a wearable sensor in some embodiments of the present disclosure.

FIG. 42 is a diagram illustrating medication targets and activation signal reference conditions corresponding to various types of wearable sensors and respective wearable sensors in some embodiments of the present disclosure.

FIG. 43 is a flowchart illustrating a process of identifying medication targets and generating an activation signal through a wearable sensor and a server in some embodiments of the present disclosure.

FIG. 44 is a flowchart illustrating a process of identifying medication targets and generating an activation signal through a wearable sensor and a server in some embodiments of the present disclosure.

FIG. 45 is a flowchart illustrating an example of operations for providing medication determination results based on at least one sensor of a wearable device according to various embodiments.

FIGS. 46 to 47 are diagrams illustrating operations of obtaining medication determination results based on sensor values of a health management system according to various embodiments.

FIGS. 48 to 50 are diagrams illustrating examples of learning an artificial intelligence model for one sensor-based medication determination and a medication determination operation based on a learned artificial intelligence model according to various embodiments.

FIG. 51 is a diagram illustrating examples of learning an artificial intelligence model for a plurality of sensors-based medication determination and a medication determination operation based on a learned artificial intelligence model according to various embodiments.

FIG. 52 is a flowchart illustrating an example of a complex medication determination operation of a wearable device according to various embodiments.

FIG. 53 is a diagram illustrating an example of a complex medication determination operation of a server according to various embodiments.

FIG. 54 is a flowchart illustrating an example of a medication determination scenario of a wearable device according to various embodiments.

FIG. 55 is a flowchart illustrating an example of a medication record and an alarm operation of a health management system according to various embodiments.

FIG. 56 is a diagram illustrating an example of an execution screen including medication determination result information according to various embodiments.

FIG. 57 is a diagram illustrating an example of handwriting input when medication is not performed according to various embodiments.

FIG. 58 is a diagram illustrating an example of medication un-authentication when medication is not performed and handwriting input is received according to various embodiments.

FIG. 59 is a flowchart illustrating an example of a medication remind operation of a health management system according to various embodiments.

FIG. 60 is a diagram illustrating an example of generating activation data for medication remind of a health management system according to various embodiments.

FIG. 61 is a flowchart illustrating an example of a multiple medication alarm operation of a health management system according to various embodiments.

FIG. 62 is a flowchart illustrating an example of a dangerous medication alarm operation of a health management system according to various embodiments.

FIG. 63 is a diagram illustrating a life log linkage operation of a health management system according to various embodiments.

FIG. 64 is a diagram illustrating an example of a life log that can be identified using a sensor of a wearable device according to various embodiments.

FIG. 65 is a flowchart illustrating an example of a medication correlation provision operation of a health management system according to various embodiments.

FIG. 66 is a diagram illustrating an example of a medication correlation identification operation of a health management system according to various embodiments.

FIG. 67 is a flowchart illustrating an example of a connection correlation providing operation of a health management system according to various embodiments.

FIG. 68 is a diagram illustrating an example of an operation for identifying a connection correlation of a health management system according to various embodiments.

FIG. 69 is a diagram illustrating an example of a connection correlation according to various embodiments.

FIG. 70 is a flowchart illustrating an example of a connection correlation providing operation of a health management system according to various embodiments.

FIG. 71 is a diagram illustrating an example of an operation of providing a service for managing a user's life log based on a correlation of a health management system according to various embodiments.

FIG. 72 is a diagram illustrating another example of an operation of providing a service for managing a user's life log based on a correlation of a health management system according to various embodiments.

FIG. 73 is a flowchart illustrating an operation method of a health management system as a digital therapeutic agent according to some embodiments of the present disclosure.

FIG. 74 is a diagram illustrating a method of providing a medication amount according to a medication after the symptom degree in some embodiments of the present disclosure.

FIG. 75 is a diagram illustrating a method of providing a medication amount according to a medication after the symptom degree in other embodiments of the present disclosure.

FIG. 76 is a diagram illustrating a method of providing a dose according to side effects after a dose according to some embodiments of the present disclosure.

FIG. 77 is a flowchart illustrating an example of an operation of providing an execution screen for a manager for managing a user's dose of a health management system according to various embodiments.

FIG. 78 is a diagram illustrating an example of an execution screen for a manager for managing a user's dose provided from a health management system according to various embodiments.

FIG. 79 is a diagram illustrating an example of an execution screen for a manager for managing a user's dose provided from a health management system according to various embodiments.

FIG. 80 is a flowchart illustrating an example of an operation for managing information of a health management system and providing information to another external server according to various embodiments.

FIG. 81 is a diagram illustrating an example of a data mining and providing method of a health management system according to various embodiments.

FIG. 82 is a flowchart illustrating an example of an operation for providing medication authentication and reward for a user of a health management system according to various embodiments.

FIG. 83 is a diagram illustrating an example of medication authentication and reward authorization of a health management system according to various embodiments.

FIG. 84 is a flowchart illustrating an example of an operation for providing medication authentication and reward for a user of a health management system according to various embodiments.

FIG. 85 is a flowchart illustrating an example of an operation for providing medication determination and reward based on medical medication of a health management system according to various embodiments.

FIG. 86 is a diagram illustrating an example of medical medication determination and reward authorization of a health management system according to various embodiments.

### DETAILED DESCRIPTION

The electronic device according to various embodiments disclosed in this document may be various types of devices. The electronic device may include, for example, a portable communication device (e.g., a smartphone), a computer device, a portable multimedia device, a portable medical device, a camera, a wearable device, or a home appliance. The electronic device according to the embodiment of this document is not limited to the above devices.

It should be understood that the various embodiments of the present disclosure and the terms used herein are not intended to limit the technical features disclosed in the present disclosure to specific embodiments, but to include various changes, equivalents, or alternatives to the corresponding embodiments. In connection with the description of the drawings, similar reference numerals may be used for similar or related elements. The singular form of a noun corresponding to an item may include one or more of the items unless the relevant context clearly indicates otherwise. In the present document, each of the phrases such as “A or B,” “at least one of A and B,” “at least one of A or B,” “A, B or C,” “at least one of A, B and C,” and “at least one of A, B, or C” may include any one of the items listed together in the corresponding phrase, or any combination of them. The terms “first,” “second,” or “first” or “second” may be used to simply distinguish the corresponding element from other corresponding elements, and the corresponding elements are not limited in other aspects (e.g., importance or order). When an element (e.g., first element) is referred to as “coupled” or “connected” together with or without the term “functionally” or “communicatively” to another element (e.g., second element), it means that the element may be connected to the other element directly (e.g., wired), wirelessly, or through a third element.

The term “module” used in various embodiments of the present disclosure may include a unit implemented in hardware, software, or firmware, and may be used interchangeably with terms such as logic, logical block, components, or circuit. The module may be an integrated component or a minimum unit of the component or a part thereof that performs one or more functions. For example, according to an embodiment, the module may be implemented in the form of an application-specific integrated circuit (ASIC).

Various embodiments of the present disclosure may be implemented as software (e.g., a program) including one or more instructions stored in a storage medium (e.g., an internal memory or an external memory) readable by a machine (e.g., an electronic device). For example, the processor (e.g., processor) of the machine (e.g., the electronic device) may invoke at least one instruction among the one or more instructions stored from the storage medium and execute it. This enables the machine to be operated to perform at least one function according to the at least one invoked instruction. The one or more instructions may include code generated by a compiler or code that can be executed by an interpreter. The storage medium readable by the machine may be provided in the form of a non-transitory storage medium. Here, “non-transitory” means that the storage medium is a tangible device and does not include a signal (e.g., electromagnetic wave), and this term does not distinguish between a case where data is semi-permanently stored in the storage medium and a case where data is temporarily stored.

According to an embodiment, the methods according to various embodiments disclosed in the present disclosure may be included and provided in a computer program product. The computer program product may be traded between a seller and a buyer as a product. The computer program product may be distributed in the form of a machine-readable storage medium (e.g., compact disc read only memory (CD-ROM)), or may be distributed (e.g., downloaded or uploaded) online via an application store (e.g., Play Store™), or directly between two user devices (e.g., smart phones). In the case of online distribution, at least a part of the computer program product may be temporarily stored or generated in a machine-readable storage medium such as a server of a manufacturer, a server of an application store, or a memory of a relay server. According to various embodiments, each component (e.g., module or program) of the above-described components may include a single entity or a plurality of entities, and some of the plurality of entities may be separately arranged in other components.

According to various embodiments, one or more components or operations among the above-described corresponding components may be omitted, or one or more other components or operations may be added. Alternatively or additionally, a plurality of components (e.g., modules or programs) may be integrated into one component. In this case, the integrated component may perform one or more functions of each component of the plurality of components in the same or similar manner as that performed by the corresponding component among the plurality of components before the integration. According to various embodiments, operations performed by a module, a program, or another component may be sequentially, in parallel, repeatedly, or heuristically executed, one or more of the operations may be executed in a different order, omitted, or one or more other operations may be added.

According to various embodiments, a device may be provided for medication determination, including: a sub device including a first fastening structure configured to be detachable from a main body, the sub device being configured to be operated independently from the main body, wherein the sub device includes: at least one processor; a short range communication circuit; and a camera disposed to an external surface of the sub device; wherein, while the sub device is coupled to the main body, the at least one processor is configured to: establish, through the short range communication circuit, a communication connection with the main body, capture an image related to a medication performed by a user using the camera, and provide the image cause the main body to obtain a result information of the medication determination.

According to various embodiments, wherein the sub device further includes housing, and a flexible substrate disposed in the housing, wherein the at least one processor, the short range communication circuit, and the camera is electrically connected through the flexible substrate.

According to various embodiments, wherein, when the sub device and the main device are combined and worn on the user's wrist, the camera is directed to photograph the user's hand,

According to various embodiments, wherein the at least one processor is configured to: receive a request for a communication connection from the main device through the short-range communication circuit, and establish a communication connection with the main device based on the received request.

According to various embodiments, wherein the at least one processor is configured to: obtain identification information about a wireless communication device physically connected to a target object for medication from the main device through the short-range communication circuit, and based on the identification information, obtain a signal containing activation data broadcast from the wireless communication device, set to activate the camera based on the activation data.

According to various embodiments, wherein the at least one processor is configured to: establish a communication connection with the wireless communication device through the short range communication circuit, based on the identification information, and obtain the signal including the activation data based on an established communication connection with the wireless communication device.

According to various embodiments, wherein the at least one processor is configured to: through the short-range communication circuit, scan a signal containing activation data broadcast from the wireless communication device based on the identification information, and based on the scan, obtain the signal including the activation data.

According to various embodiments, wherein the at least one processor is configured to: transmit the captured image to the outside based on activation of the camera through the short range communication circuit, wherein, the main device is configured to to obtain result information of the medication determination, based on the transmitted image being analyzed by at least one artificial intelligence model of the server.

According to various embodiments, an operation method of device may be provided for medication determination including a main device and a sub device configured to be detachable from the main body, comprising: establishing, through the short range communication circuit, a communication connection with the main body; capturing an image related to a medication performed by a user using the camera, and providing the image cause the main body to obtain a result information of the medication determination.

According to various embodiments, wherein the sub device further includes housing, and a flexible substrate disposed in the housing, wherein the at least one processor, the short range communication circuit, and the camera is electrically connected through the flexible substrate.

According to various embodiments, wherein, when the sub device and the main device are combined and worn on the user's wrist, the camera is directed to photograph the user's hand, According to various embodiments, wherein the method further comprises: receiving a request for a communication connection from the main device through the short-range communication circuit, and establishing a communication connection with the main device based on the received request.

According to various embodiments, wherein the method further comprises: obtaining identification information about a wireless communication device physically connected to a target object for medication from the main device through the short-range communication circuit, and based on the identification information, obtaining a signal containing activation data broadcast from the wireless communication device, set to activate the camera based on the activation data.

According to various embodiments, wherein the method further comprises: establishing a communication connection with the wireless communication device through the short range communication circuit, based on the identification information; and obtaining the signal including the activation data based on an established communication connection with the wireless communication device.

According to various embodiments, wherein the method further comprises: through the short-range communication circuit, scanning a signal containing activation data broadcast from the wireless communication device based on the identification information, and based on the scan, obtaining the signal including the activation data.

### 1. An Overview of Health Management System 1

According to various embodiments, the Health Management System 1 may be a system implemented to provide health management data collected by automatically recording and analyzing various kinds of health management behaviors of the user by using the wearable device 20 that can be worn by the user. The Health Management System 1 may be implemented to contribute to promoting human health, by conveniently recording the health management behavior even in unconsciousness without requiring the user to consciously record the health management behavior, and by providing health management data to the user that can provide insights to health management based on analyzing the health management behavior using an artificial intelligence (AI) model.

### 1.1 Components of the Health Management System 1

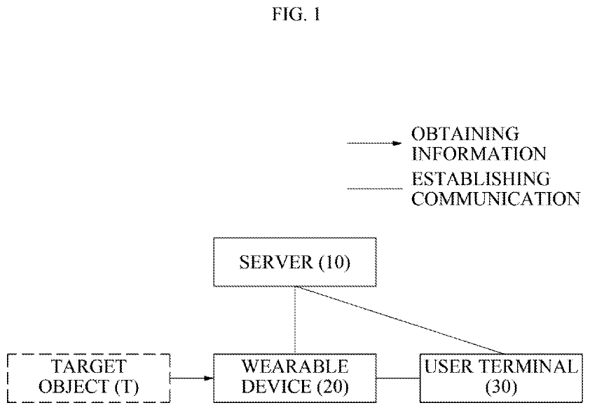

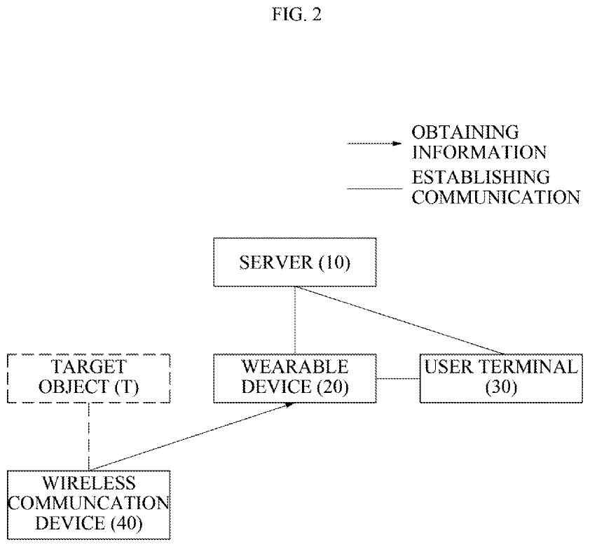

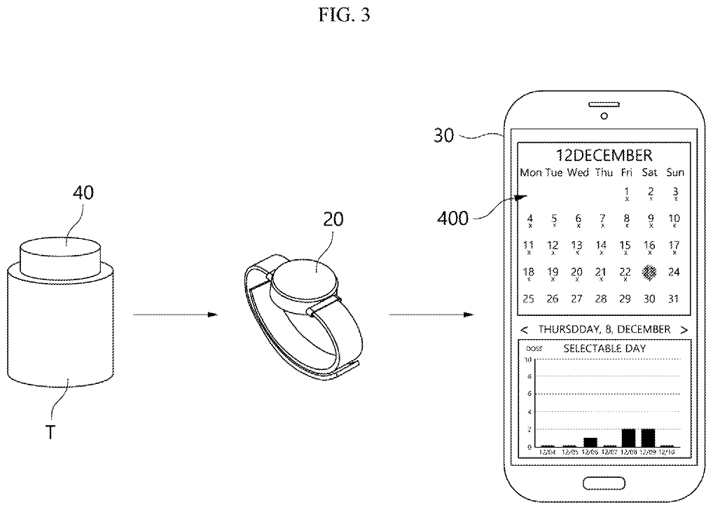

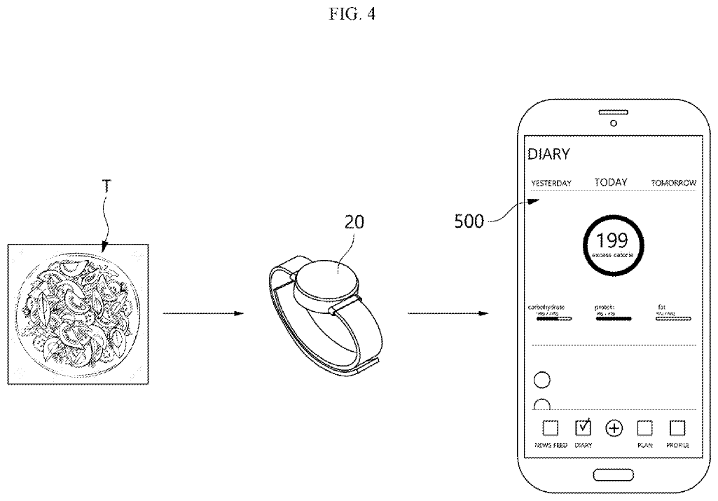

FIG. 1 is a block diagram illustrating an example of a configuration of the health management system 1 according to various embodiments. FIG. 2 is a block diagram illustrating another example of a configuration of the health management system 1 according to various embodiments. Hereinafter, FIGS. 1 to 2 will be further described with reference to FIGS. 3 to 4.

FIG. 3 is a diagram illustrating an example of a health management behavior according to various embodiments. FIG. 4 is a diagram illustrating another example of a health management behavior according to various embodiments.

According to various embodiments, referring to FIG. 1, the health management system 1 may include a server 10, a wearable device 20, a user terminal 30, and a wireless communication device 40 disposed (or provided) in a form related to a target object T associated with the health management behavior (e.g., attached, coupled, etc.). For example, referring to FIGS. 3 to 4, the health management behavior analyzed by the health management system 1 may include a medication behavior and a diet (or diet) management behavior, and is not limited to the illustrated and/or described examples, and may include various types of health management behaviors for promoting the health of a user such as exercise and sleep. Meanwhile, the health management system 1 may be implemented to further include more components and/or include fewer components, without being limited to the illustrated and/or described examples of FIG. 1. For example, referring to FIG. 2, the health management system 1 may be implemented to obtain information on the health management behavior of the user of the health management system I using only the wearable device 20, without implementing the wireless communication device 40. For example, although not shown, the health management system 1 may be implemented to provide health management data to the user through another device (e.g., the wearable device 20) without implementing the user terminal 30.

According to various embodiments, the server 10 may be implemented to analyze information on health management behavior and provide a service for health management. For example, the server 10 may obtain information on health management behavior received from the wearable device 20 and provide health management data generated based on analyzing the information on health management behavior to the user. For example, the health management data may be a broad concept including the contents of health management behavior, the quality of health management behavior, and the like, as well as whether the user performs health management behavior. Based on the health management data provided from the server 10, the user may perform the health management behavior more efficiently. For example, referring to FIG. 3, the server 10 may obtain information on medication behavior from the wearable device and provide information on whether the medication behavior is performed (medication determination result information) to the user through the user terminal 30 as the health management data, so that the user may manage the medication management behavior. Information on reliability along with the medication determination result information may also be provided, which will be described later.

According to various embodiments, the wearable device 20 may be implemented to automatically (e.g., regardless of the control of the user) acquire information about health management behavior. For example, the information about health management behavior includes an image (or image) obtained by photographing a health management behavior, a sensor value generated during the health management behavior, and the like, but is not limited to the examples described, and may include various types of information for recording the health management behavior when the user performs the health management behavior. For example, referring to FIGS. 3 to 4, the wearable device 20 may acquire an image of the medication behavior performed by the user and/or an image of the diet taken by the user. Referring to FIG. 3, the wearable device 20 may be implemented to initiate an operation (e.g., image photographing using a camera) for acquiring the information about health management behavior based on activation data received through the wireless communication device 40 attached to the target object T for the health management behavior. The target object T means an object associated with the health management behavior, and may be different depending on the type of the health management behavior. For example, in the case of the medication behavior, the target object T may include a medication target (e.g., a drug bottle), a hand of the user, and the like, but is not limited to the examples described. For example, in the case of the diet, the target object T may include a dish, a hand of the user, and the like, but is not limited to the examples described. As the operation of acquiring the information about health management behavior of the wearable device 30 is automatically triggered based on the wireless communication device 40 attached to the target object T, the burden of the operation of the wearable device 30 for the health management behavior may be reduced, and the acquisition of all health management behaviors may be automatically enabled without the user's intervention. On the other hand, the wearable device 20 may be implemented to acquire activation data based on a user's input (e.g., a touch input for photographing), and initiate an operation (e.g., image photographing using a camera) for acquiring information on the health management behavior without receiving activation data from the wireless communication device 40.

According to various embodiments, the user terminal 30 may provide the health management data to the user. For example, based on the execution of the application installed in the user terminal 30, the user terminal 30 may display the execution screens 300 and 400 including the health management data to provide information to the user. For example, referring to FIGS. 3 to 4, the execution screens 300 and 400 may include information on whether to perform the medication action and/or information on the constituent components of the diet, but are not limited to the illustrated and/or described examples, and may include various types of information based on the analysis results of the health management behavior. However, the present invention is not limited to the illustrated and/or described examples, and the health management data may be provided to the user in various forms such as audible, tactile, and other forms as well as visual content.

According to various embodiments, the wireless communication device 40 may be implemented to activate the operation of obtaining the health management behavior information of the wearable device 20 by providing a wireless signal to the wearable device 20. For example, referring to FIG. 3, the wireless communication device 40 may be attached to the target object T for the health management behavior, and may be implemented to generate an activation signal for inducing the operation of obtaining the health management behavior information of the wearable device 20 based on the sensor value naturally generated according to the change of the target object T when the user performs the health management behavior, and transmit the activation signal to the wearable device 20.

Meanwhile, for convenience of explanation, the types of health management behaviors managed by the health management system I are specified and described as “medication actions”, but it is obvious to those skilled in the art that the contents described below may be applied to other types of health management behaviors unless otherwise noted.

### 1.1.1 the Implementation Type of the Health Management System 1

According to various embodiments, the health management system 1 may be implemented as a server type implemented such that analysis of information on health management behaviors is intensively performed at the server 10 for a health management service, or an on-device type that may be performed at a local device (e.g., the wearable device and/or the user terminal 30) other than the server 10.

In an embodiment, when the health management system 1 is implemented as a server type, the health management system 1 may be implemented such that information on health management behaviors collected by the wearable device 20 at the server 10 is analyzed, as described above with reference to FIGS. 1 to 2.

Unlike the illustrated and/or described embodiments, when the health management system 1 is implemented as an on-device type, operations performed at the server 10 described below may all be performed at the local device (e.g., the wearable device 20 and/or the user terminal 30). For example, as the medication determination module 500 of the server 10 is stored at the local device (e.g., the wearable device 20 and/or the user terminal 30), analysis of information on medication behaviors and determination of whether to perform medication may be performed at the local device (e.g., the wearable device 20 and/or the user terminal 30).

### 1.2. Components of the Health Management System 1

Hereinafter, an example of components of the Health Management System 1 according to various embodiments will be described.

### 1.2.1. Components of the Server 10

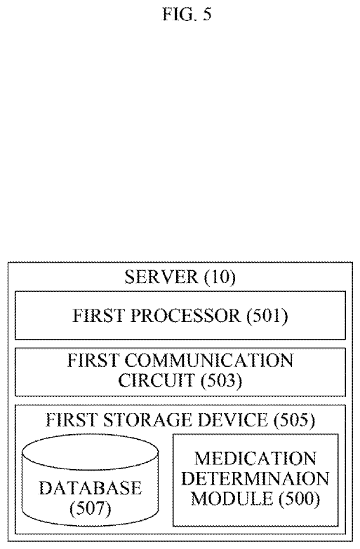

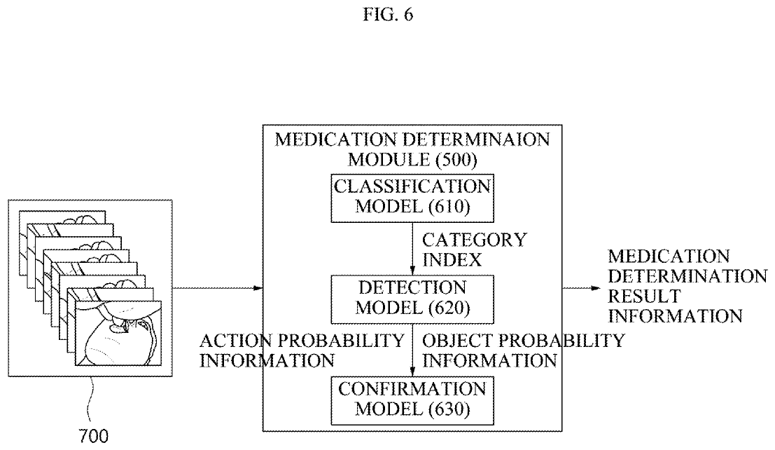

FIG. 5 is a block diagram illustrating an example of a component of the server 10 according to various embodiments. Hereinafter, FIG. 5 will be further described with reference to FIGS. 6 to 8.

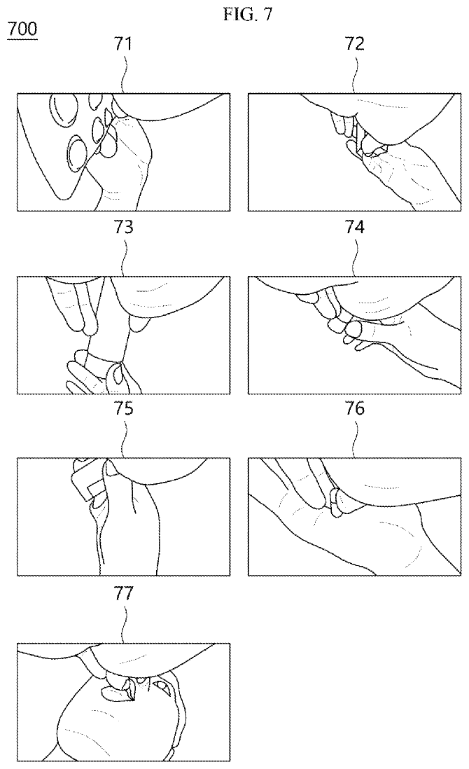

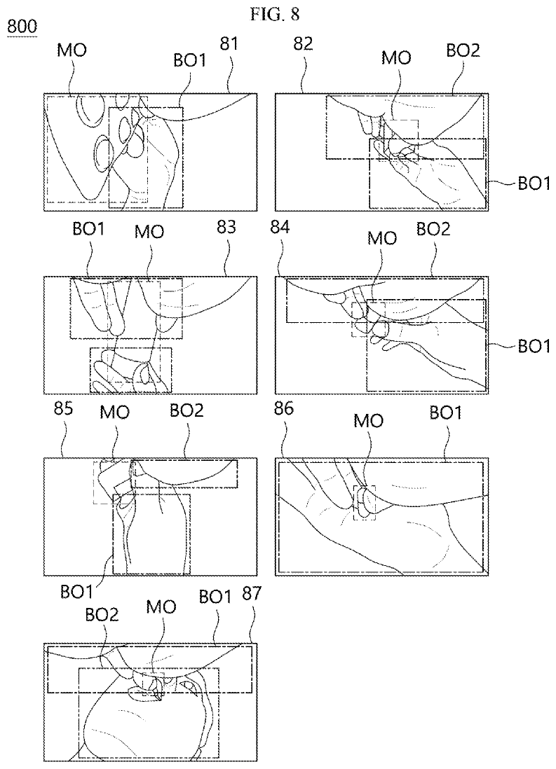

FIG. 6 is a diagram for explaining an example of at least one artificial intelligence model for obtaining health management data for a medication behavior according to various embodiments. FIG. 7 is a diagram for explaining an example of a medication behavior according to various embodiments. FIG. 8 is a diagram for explaining an example of an image obtained by photographing a medication behavior as training data for at least one artificial intelligence model according to various embodiments.

According to various embodiments, referring to FIG. 5, the server 10 may include a first processor 501, a first communication circuit 503, and a first storage device 505 that stores a database 507 and a medication determination module 500. However, the server 10 may be implemented to include more components and/or fewer components, without being limited to the illustrated and/or described examples.

According to various embodiments, the first processor 501 may control at least one other component (e.g., hardware or software component) of the server 10 connected to the first processor 501 by executing software (e.g., the medication determination module 500), and may perform various data processing or calculations. According to an embodiment, as at least a part of the data processing or calculation, the first processor 501 may store the command or data received from the other component (e.g., the first communication circuit 503) in a volatile memory, process the command or data stored in the volatile memory, and store the resultant data in a non-volatile memory. According to an embodiment, the first processor 501 may include a main processor (not shown) (e.g., a central processing unit or an application processor) or an auxiliary processor (not shown) (e.g., a graphic processing unit, a neural processing unit (NPU), an image signal processor, a sensor hub processor, or a communication processor) that can operate independently or together with the main processor (not shown). For example, when the server 10 includes the main processor (not shown) and the auxiliary processor (not shown), the auxiliary processor (not shown) may be set to use lower power than the main processor (not shown) or to be specific to a specified function. The auxiliary processor (not shown) may be implemented separately from the main processor (not shown) or as a part thereof.

For example, the auxiliary processor (not shown) may control at least a part of a function or state related to at least one component (e.g., the first communication circuit 503) of the components of the server 10 instead of the main processor (not shown) while the main processor (not shown) is in an inactive (e.g., sleep) state, or together with the main processor (not shown) while the main processor (not shown) is in an active (e.g., application execution) state. According to an embodiment, the auxiliary processor (not shown) (e.g., an image signal processor or a communication processor) may be implemented as a part of another component (e.g., the first communication circuit 503) that is functionally related. According to an embodiment, the auxiliary processor (not shown) (e.g., a neural network processing device) may include a hardware structure specialized for processing an artificial intelligence model. The artificial intelligence model may be generated through machine learning. Such learning may be performed, for example, in the server 10 itself in which artificial intelligence is performed, or may be performed through a separate server (e.g., a learning server). The learning algorithm may include, for example, supervised learning, unsupervised learning, semi-supervised learning, or reinforcement learning, but is not limited to the above example. The artificial intelligence model may include a plurality of artificial neural network layers. The artificial neural network may be one of a deep neural network (DNN), a convolutional neural network (CNN), a recurrent neural network (RNN), a restricted boltzmann machine (RBM), a deep belief network (DBN), a bidirectional recurrent deep neural network (BRDNN), a deep Q-network, or a combination of two or more of the above, but is not limited to the above examples. The artificial intelligence model may additionally or alternatively include a software structure in addition to the hardware structure.

According to various embodiments, the first communication circuit 503 may support the establishment of a direct (e.g., wired) communication channel or a wireless communication channel between the server 10 and the external electronic device (e.g., the wearable device 20, the user terminal 30) and communication performance through the established communication channel. For example, the server 10 may receive the medication image captured by the wearable device 20 through the first communication circuit 503. The first communication circuit 503 may include one or more communication processors that are operated independently from the first processor 501 (e.g., the application processor) and support direct (e.g., wired) communication or wireless communication. According to an embodiment, the first communication circuit 503 may include a wireless communication module (not shown) (e.g., a cellular communication module, a short-range wireless communication module, or a global navigation satellite system (GNSS) communication module) or a wired communication module (e.g., a local area network (LAN) communication module, or a power line communication module). Among these communication modules, a corresponding communication module may communicate with the external electronic device (e.g., the wearable device 20, the user terminal 30) through a first network (e.g., a short-range communication network such as Bluetooth, wireless fidelity (WiFi) direct, or infrared data association (IrDA)) or a second network (e.g., a long-range communication network such as a legacy cellular network, a 5G network, a next-generation communication network, the Internet, or a computer network (e.g., LAN or WAN)). These various types of communication modules may be integrated into one component (e.g., a single chip), or may be implemented as a plurality of separate components (e.g., a plurality of chips). The wireless communication module may identify or authenticate the server 10 in a communication network such as the first network or the second network using subscriber information (e.g., an international mobile subscriber identifier (IMSI)) stored in the subscriber identification module.

According to various embodiments, the first storage device 505 may store various data used by at least one component (e.g., the processor 110 or the sensor module 176) of the server 10. The data may include, for example, software (e.g., programs and input data or output data for commands related thereto). The first storage device 505 may include a volatile memory or a non-volatile memory.

According to various embodiments, the first storage device 505 may be implemented to store the medication determination module 500 and the database 507. However, the first storage device 505 may be implemented to further store various data, information, and/or software necessary for the operation of the server 10, without being limited to the illustrated and/or described examples.

According to various embodiments, the medication determination module 500 may be software (or computer program, computer code, or instructions) implemented to provide health management data based on analyzing information related to medication. Referring to FIG. 7, the medication determination module 500 may include at least one artificial intelligence model. The at least one artificial intelligence model may include at least one of a classification model 610, a detection model 620, or a confirmation model 630, and at least one of the artificial intelligence models shown may be omitted without being limited to the example shown. The at least one artificial intelligence model (e.g., at least one of the classification model 610, the detection model 620, or the confirmation model 630) may be trained to output specific information based on at least one image (or image) obtained by capturing an operation 700 performed sequentially (or time seriesly) when medication is performed, as shown in FIG. 7. Illustratively referring to FIG. 8, the sequentially performed operation 700 may include an operation of holding a medication-related object such as a blister 71, an operation of holding an inhaler 72, an operation of holding a turbohaler 73, an operation of holding a pill bottle 74, an operation of holding a nasal spray 75, an operation of holding a pill 76, an operation of taking a drug such as a pill 77, an operation of inhaling a drug using a medication-related object such as an inhaler, a turbohaler, a nasal spray, an operation of administering a pill using a medication-related object such as a pill bottle, an operation of injecting a drug using a medication-related object such as a syringe, and an operation of opening a medication-related object such as a pill. Since the learning algorithm for learning is the same as described above, redundant descriptions will be omitted.

According to various embodiments, the classification model 610 may be implemented to identify a category of at least one image (or image) 600 (hereinafter referred to as a medication image) associated with the medication captured by the wearable device 20. The category of the medication image 600 represents the type of medication, the medication target, etc., and examples thereof include, but are not limited to, a drug dose category, an inhaler use category, a turbohaler use category, a nasal spray use category, an eye drop administration category, a drug injection category, etc. The classification model 610 may be implemented to output a category index indieating the category in response to receiving the medication image 600. Since the learning algorithm for learning is the same as described above, redundant descriptions will be omitted.

According to various embodiments, result information output from the detection model 620 described below may be different based on the category index. In an embodiment, as different detection models 620 are implemented for each category index, information associated with the target object T acquired by inputting the medication image to the detection model 620 selected by the category index may vary. In another embodiment, as the detection model 620 is implemented so that the category index can be input together with the medication image 600 to the detection model 620, information associated with the target object T acquired from the detection model 620 selected by the category index may vary.

According to various embodiments, the detection model 620 may be implemented to detect information associated with the target object T of the medication image 600. The information associated with the target object T may include at least one of probability information on the object associated with the medication or probability information on at least one of the operations associated with the medication. The object associated with the medication means a product containing pill packaging material such as a pain, a press through package (PTP) and the like, an inhaler, a turbohaler, an eye drop, and the like, and is not limited to the examples described. In addition, the operations associated with the medication may mean the operation 700 sequentially performed during the medication described above and/or may mean the body part associated with the operation 700 sequentially performed during the medication. For example, when the operation is the action of holding the pill (76), as shown in FIG. 8, the body part associated with the operation 700 sequentially performed during the medication may mean the palm (B01). The detection model 620 may be implemented to output at least one of probability information on the sequential (or time series) or the at least one of the operations associated with the medication or the probability information on the object associated with the medication in response to receiving the medication image 600 or the at least one of the category indexes.

According to various embodiments, the detection model 620 may be trained based on training data 800 including operations 700 that are sequentially performed and images 800 captured for each operation 700 that are sequentially performed, as shown in FIG. 8. For example, the images 81, 82, 83, 84, 85, 86, 87 for each operation include objects MO that are subject to medication included in the images for each operation and/or parts B01 and B02 of the body for medication, and the objects MO and parts B01 and B02 of the body may be labeled for each of the images 81, 82, 83, 84, 85, 86, 87. The server 10 may set the operations 700 as output data, and set the labeled images 800 corresponding to each operation 700 as input data to acquire the detection model 620 as the learning is performed.

According to various embodiments, the confirmation model 630 may be implemented to acquire information on whether to perform medication. For example, the confirmation model 630 may provide result information indieating whether to perform medication (e.g., medication, non-medication) in response to receiving the information associated with the target object T output from the detection model 620. The confirmation model 630 may be implemented to additionally provide reliability information on the result information, and the reliability may be provided as a reliability value of 0% or more and 100% or less of the result information, but is not limited to the examples described.

Various embodiments of the present disclosure may be implemented as software (e.g., a program) including one or more instructions stored in a storage medium (e.g., an internal memory or an external memory) readable by a machine. For example, the processor (e.g., the first processor 501) of the machine (e.g., the server 10) may invoke at least one instruction among the one or more instructions stored from the storage medium and execute it. This enables the machine to be operated to perform at least one function according to the at least one invoked instruction. The one or more instructions may include code generated by a compiler or code that can be executed by an interpreter. The storage medium readable by the machine may be provided in the form of a non-transitory storage medium. Here, the term “non-transitory” merely means that the storage medium is a tangible device and does not include a signal (e.g., electromagnetic wave), and this term does not distinguish between the case where data is semi-permanently stored in the storage medium and the case where data is temporarily stored.

According to an embodiment, the methods according to various embodiments disclosed in the present disclosure may be included and provided in a computer program product. The computer program product may be traded between a seller and a buyer as a product. The computer program product may be distributed in the form of a machine-readable storage medium (e.g., compact disc read only memory (CD-ROM)), or may be distributed (e.g., downloaded or uploaded) online through an application store (e.g., Play Store™), or directly between two user devices (e.g., smart phones). In the case of online distribution, at least a part of the computer program product may be temporarily stored or generated in a machine-readable storage medium such as a server of a manufacturer, a server of an application store, or a memory of a relay server. According to various embodiments, each component (e.g., module or program) of the above-described components may include a single entity or a plurality of entities, and some of the plurality of entities may be separately arranged in other components.

According to various embodiments, one or more components or operations among the above-described corresponding components may be omitted, or one or more other components or operations may be added. Alternatively or additionally, a plurality of components (e.g., modules or programs) may be integrated into one component. In this case, the integrated component may perform one or more functions of each component of the plurality of components in the same or similar manner as that performed by the corresponding component among the plurality of components before the integration. According to various embodiments, operations performed by a module, a program, or another component may be executed sequentially, in parallel, repeatedly, or heuristically, one or more of the operations may be executed in a different order, omitted, or one or more other operations may be added.

### 1.2.2 Components of the Wearable Device 20

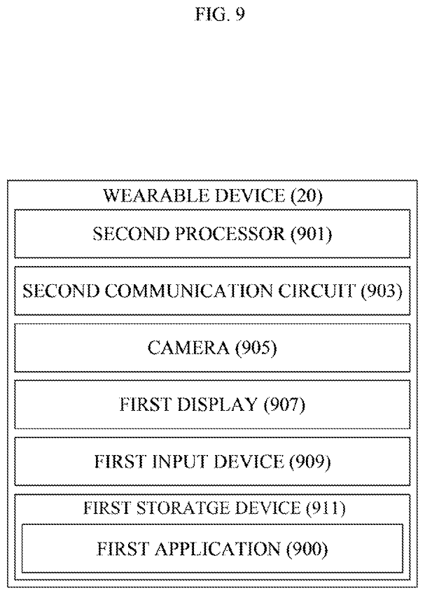

FIG. 9 is a block diagram illustrating an example of components of the wearable device according to various embodiments.

According to various embodiments, referring to FIG. 9, the wearable device 20 may include a second processor 901, a second communication circuit 903, a camera 905, a first display 907, a first input device 909, and a second storage device 911. The wearable device 20 may be implemented to include more components and/or fewer components, without being limited to the illustrated and/or described examples. Since the second processor 901 may be implemented as the first processor 501, the second communication circuit 903 may be implemented as the first communication circuit 503, and the second storage device 911 may be implemented as the first storage device 505, redundant descriptions will be omitted.

According to various embodiments, the camera 905 may be implemented to capture at least one image (or image). For example, the camera 905 may be implemented to capture a health management behavior. The camera 905 may be activated based on the control of the second processor 901. The second processor 901 may be implemented to activate the camera 905 based on an activation signal received from the wireless communication device 40, as described above. At this time, the second processor 901 may be further set to activate the camera 905 when a specific condition is satisfied when the activation signal is received, but is not limited thereto. For example, the second processor 901 may be set to determine that the specific condition is satisfied when the received signal strength indication (RSSI) of the received activation signal is equal to or greater than a specific value, and activate the camera 905.

According to various embodiments, the first display 907 may visually provide information to the outside (e.g., a user) of the wearable device 20. The display module 160 may include, for example, a display, a hologram device, or a projector, and a control circuit for controlling the corresponding device. According to an embodiment, the first display 907 may include a touch sensor configured to sense a touch or a pressure sensor configured to measure the intensity of force generated by the touch (e.g., including a touch screen).

According to various embodiments, the first input device 909 may be implemented to acquire a user input received by the wearable device 20. For example, the user input may include a tactile input (e.g., touch, press), an audible input (e.g., a user's utterance), and the like. Accordingly, the first input device 909 may include a physical button for acquiring a tactile input (e.g., a user's press), a microphone for acquiring an audible input (e.g., a user's utterance), and the like, but is not limited to the described example.

According to various embodiments, the second storage device 911 may store the first application 900. The first application 900 may control to perform at least one function to provide a medication management service through the wearable device 20. For example, the at least one function may include at least one of a function of activating the camera 905 of the wearable device 20 to photograph a medication action, a function of setting a communication connection with an external device (e.g., the user terminal 30 or the wireless communication device 40) different from the wearable device 20, a function of providing the photographed medication image 600 to another external device (e.g., the user terminal 30 or the wireless communication device 40), or a function interworking with the second application 1000 installed in the user terminal 30, which will be described later. The first application 900 may be downloaded from the server 10 (or a separate app distribution server).

### 1.2.3. Components of the User Terminal 30

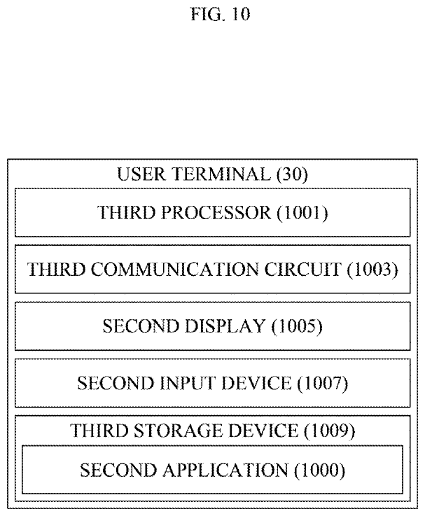

FIG. 10 is a block diagram illustrating an example of components of the user terminal according to various embodiments.

According to various embodiments, referring to FIG. 10, the user terminal 30 may include a third processor 1001, a third communication circuit 1003, a second display 1005, a second input device 1007, and a third storage device 1009. The user terminal 30 may be implemented to include more components and/or fewer components, without being limited to the illustrated and/or described examples. Since the third processor 1001 is implemented as the first processor 501, the third communication circuit 1003 is implemented as the first communication circuit 503, the second display 1005 is implemented as the first display 907, the second input device 1007 is implemented as the first input device 909, and the third storage device 1009 is implemented as the first storage device 505, redundant descriptions are omitted.

According to various embodiments, the third storage device 1009 may store the second application 1000. The second application 1000 may be implemented such that the user terminal provides health management data to the user. For example, the execution screen of the second application 1000 may include information on the health management data. For example, the execution screen of the second application 1000 may be implemented to include the result of the user's medication determination. The second application 1000 may be downloaded from the server 10 (or a separate app distribution server).

### 1.2.4. Components of a Wireless Communication Device 40

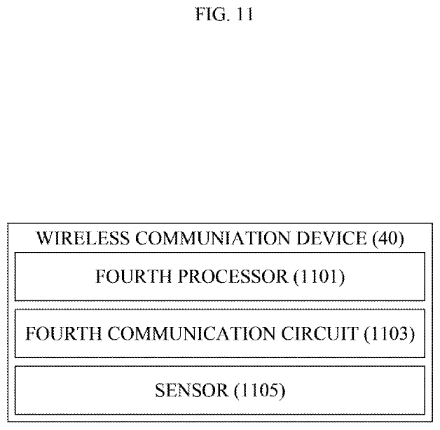

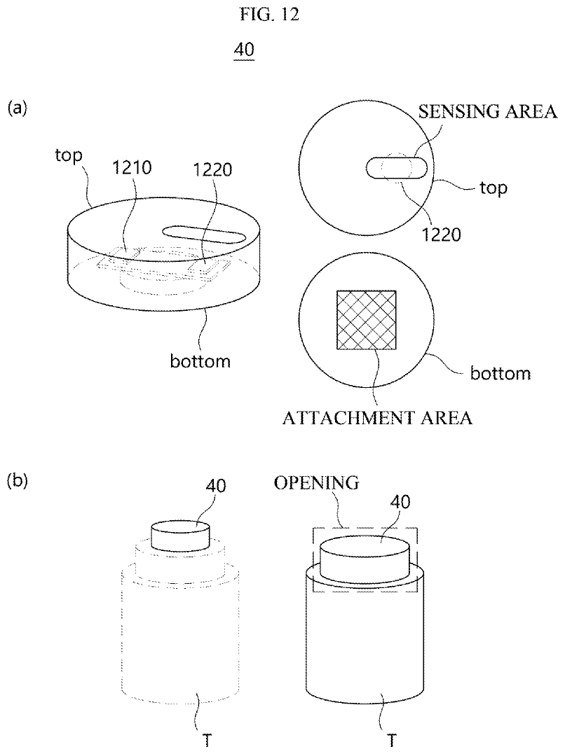

FIG. 11 is a block diagram illustrating an example of components of a wireless communication device 40 according to various embodiments. FIG. 12 is a diagram for explaining an example of a wireless communication device 40 attached to a target object T according to various embodiments.

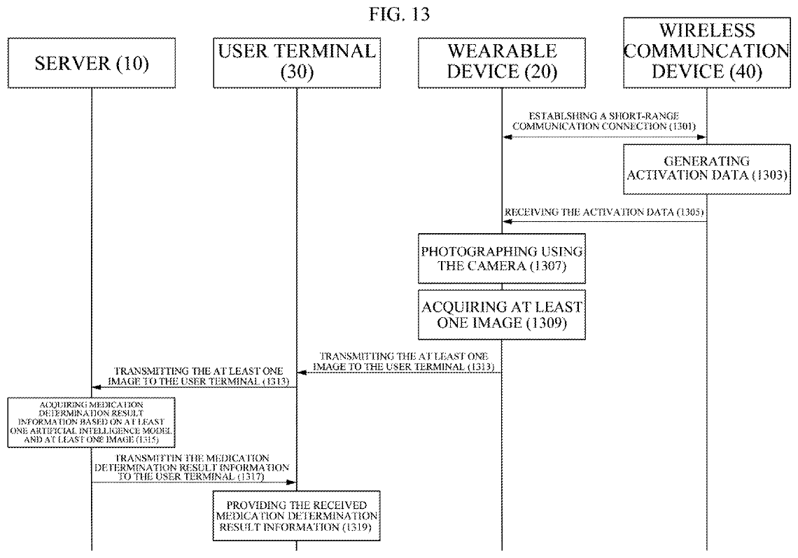

According to various embodiments, referring to FIG. 11, the wireless communication device 40 may include a fourth processor 1101, a fourth communication circuit 1103, and a sensor 1105. The wireless communication device 40 may be implemented to include more components and/or fewer components, without being limited to the illustrated and/or described examples. For example, referring to FIG. 13, the wireless communication device 40 may be attached to the target object T based on an adhesive member for attachment. Meanwhile, the fourth processor 1101 may be implemented as the first processor 501 described above, and the fourth communication circuit 1103 may be implemented as the first communication circuit 503 described above, so redundant descriptions are omitted.

According to various embodiments, the wireless communication device 40 may include a housing for contacting the target object T. The housing may include an upper surface, a lower surface, a side surface connected between the upper surface and the lower surface, and an internal space defined by the upper surface, the lower surface, and the side surface. Referring to FIG. 12A, the housing may be implemented in a cylindrical shape, but is not limited to the illustrated shape, and may be implemented in various shapes. As illustrated in FIG. 12B, the above-described adhesive member may be provided on the lower surface of the housing and attached to the target object T (e.g., a medicine bottle), but is not limited to the illustrated example, and the adhesive member may be implemented in various shapes such as a pocket attached to the target object T in the form of putting the target object T.

According to various embodiments, the sensor 1105 may be implemented to sense the initiation of the performance of the medicine of the user. For example, the sensor 1105 may include at least one of a motion sensor 1210 (e.g., a tilt sensor, an angular velocity sensor) or an illuminance sensor 1220. The sensor 1105 may be provided in the internal space of the housing, and an opening may be formed in a part of the upper surface and/or the lower surface of the housing corresponding to the illuminance sensor, so that external light may be disposed in the internal space of the housing. Accordingly, the fourth processor 1101 may determine whether to generate an activation signal based on at least one of the illuminance value identified by the illuminance sensor or the motion value (e.g., the tilt value, the angular velocity value) identified by the motion sensor. For example, when at least one of the illuminance value or the motion value satisfies a specific value (or is equal to or greater than a specific value), the activation signal may be determined to be generated. The activation signal may enable the wearable device 20 to activate the camera 905.

According to various embodiments, the fourth communication circuit 1103 may establish a communication connection with the external device (e.g., the wearable device 20). The fourth processor 1101 may transmit the generated activation signal to the wearable device through the fourth communication circuit 1103.

### 2.1. Basic Operations of the Health Management System 1

FIG. 13 is a flowchart illustrating an example of basic operations for medication behavior, which is an example of health management behavior of the Health Management System 1, according to various embodiments. Operations may be performed regardless of the order of the operations shown and/or described, and more operations may be performed and/or fewer operations may be performed.

According to various embodiments, the wearable device 20 and the wireless communication device 40 may establish a short-range communication connection in operation 1301. For example, the short-range communication connection may be a communication connection based on Bluetooth, BLE, Wi-Fi direct, and/or NFC.

According to various embodiments, the wireless communication device 40 may generate activation data in operation 1303. For example, when the user holds the target object T (e.g., a medicine pain) for medication, a movement of the wireless communication device 40 attached to the target object T may occur. Based on the movement, the wireless communication device 40 may acquire at least one sensing value from the sensor 1105 (e.g., the motion sensor 1210 and the illuminance sensor 1220), generate the activation signal when the acquired at least one sensing value satisfies a predetermined condition, and transmit the generated activation signal to the wearable device 20 based on the short-range communication connection.

According to various embodiments, the wearable device 20 may receive the activation data in operation 1305, photograph using the camera 905 in operation 1307, acquire at least one image (or video) in operation 1309, and transmit the at least one image to the user terminal in operation 1311. The wearable device 20 may acquire a medication image 600 including at least one image for each of the sequentially performed medication operations by activating the camera 905 based on the activation data, and photographing the user's medication behavior for a pre-set time (hereinafter, a photographing time) based on the activated camera 905. Alternatively, the medication image 600 acquired may not include the medication operation when the user does not perform the medication behavior, but is not limited to the described example. The wearable device 20 may determine whether the RSSI of the activation data is equal to or greater than (or exceeds a pre-set value) as at least a part of the operation of activating the camera 905 based on the activation data, and activate the camera 905 when the RSSI is equal to or greater than the pre-set value, but is not limited to the described example. The transmission of the photographed medication image 600 of the wearable device 20 may be performed in real time at the same time as the photographing of the camera 905, and/or may be performed after the camera 905 is deactivated after the photographing time.

According to various embodiments, the transmission of the medication image 600 may be performed via the user terminal 30 (hereinafter, via transmission). For example, the wearable device 20 may transmit the medication image 600 to the user terminal 30 based on a short-range communication connection, and the user terminal 30 may transmit the received medication image 600 to the server 10 based on a long-range communication (e.g., Wi-Fi and/or cellular communication) in operation 1313. The via transmission may be set to be performed under a specific condition, but is not limited to the described example. For example, the via transmission may be set to be triggered when the communication connection setting between the wearable device 20 and the server 10 is released and/or when the quality of the communication connection setting is less than a threshold.

According to various embodiments, the server 10 may acquire medication determination result information based on at least one artificial intelligence model (e.g., the classification model 610, the detection model 620, and the confirmation model 630) and at least one image in operation 1315, and transmit the medication determination result information to the user terminal 30 in operation 1317. For example, in response to inputting the received at least one image to the at least one artificial intelligence model (e.g., the classification model 610, the detection model 620, and the confirmation model 630), the server 10 may acquire medication determination result information indieating whether the medication and the reliability of the medication determination result information.

According to various embodiments, in operation 1319, the user terminal 30 may provide the received medication determination result information. For example, the user terminal 30 may display an execution screen including the received medication determination result information. When the second application 1000 is executed by the user, the user terminal may display the execution screen of the above-described second application 1000 including the medication determination result information and/or display a lock screen (or pop-up panel) including the medication determination result information based on the execution of the second application 1000.

### 2.2. Camera Shielding Structure

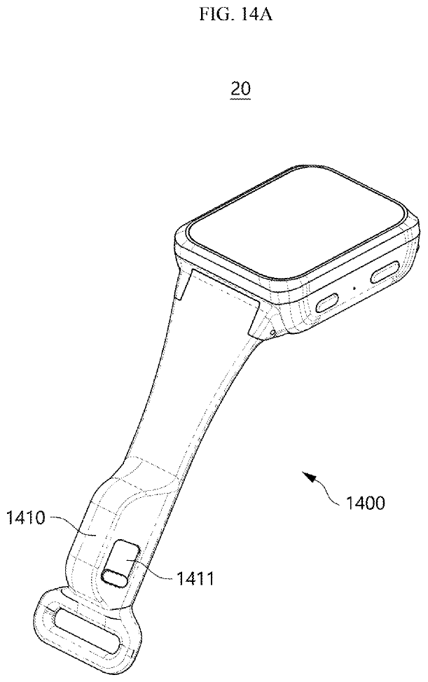

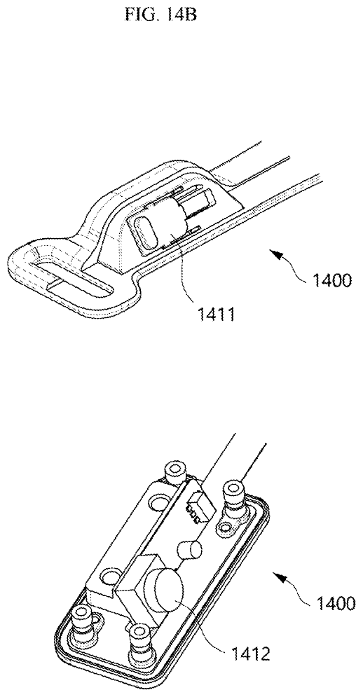

FIG. 14A is a perspective view illustrating a wearable device according to some embodiments of the present disclosure, and FIG. 14B is a perspective view illustrating a camera and a protective cover in a state in which a housing of a main strap is removed in some embodiments of the present disclosure.

Referring to FIGS. 14A and 14B, the wearable device 20 includes a main body that visually provides a user interface to the user through the first display 1007, and a strap that is worn coupled to the body of the user. In various embodiments, the strap may include a main strap 1400 that is detachably coupled to one side of the main body and includes a camera 1005 for capturing a medication action of the user, and a sub strap (not shown) that includes a length adjusting portion that is detachably coupled to the other side of the main body.

The main strap 1400 and the sub strap may be coupled to each other and worn on the body of the user, and are not limited to specific coupling methods such as gap fitting, interference fitting, or magnet coupling, and various types of coupling methods may be applied. The camera 1005 provided at one side of the main strap 1400 may form a predetermined angle so that the capturing of the medication action of the user is easy, and there is no interference with the body of the user when the wearable device 20 is worn.

Meanwhile, as shown in FIG. 14A, the main strap 1400 may include a housing 1410 that surrounds the camera 1005 to protect the camera 1005 from impact, and a protective cover 1411 that is provided at one side of the housing 1410 to prevent seating of foreign substances or the like into the camera lens.

Referring to FIG. 14B, the protective cover 1411 is illustrated as a sliding structure, but may also be implemented as a manual shielding structure that allows the user to directly open the protective cover 1411 using an external force as well as an automatic shielding structure using an electrochromic element or the like. In the case of the automatic shielding structure, the on/off of the protective cover 1411 may be controlled through a shielding device and a control module connected to the protective cover 1411.

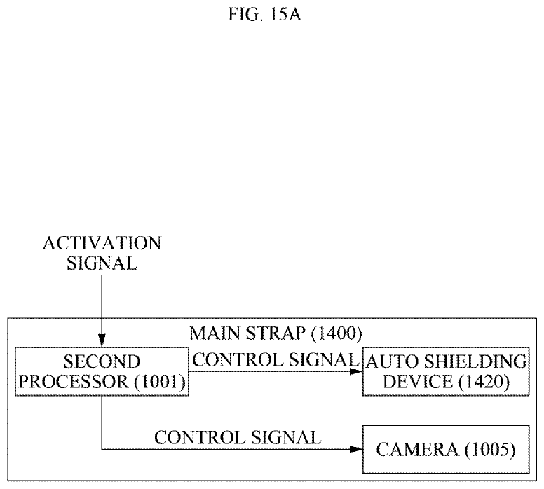

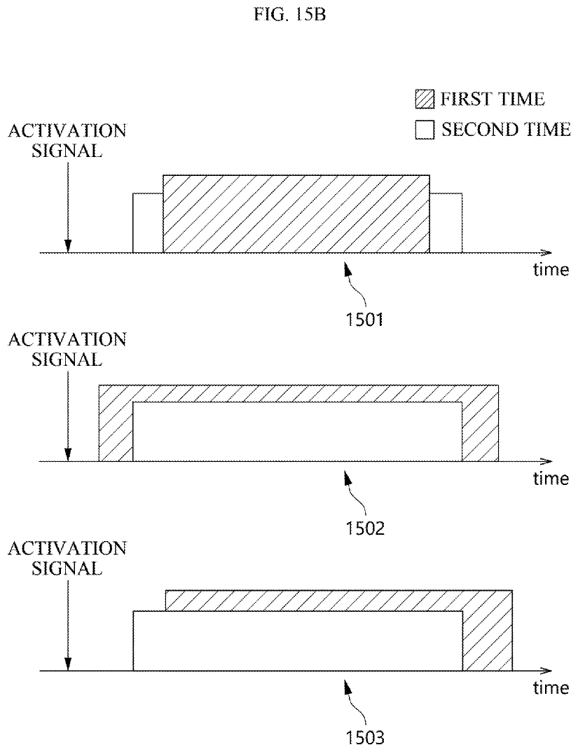

FIG. 15A is a diagram illustrating the automatic shielding structure in some embodiments of the present disclosure, and FIG. 15B is a diagram illustrating the operation order of the automatic shielding protective cover and the camera in some embodiments of the present disclosure. Hereinafter, the operation method of the protective cover will be described with reference to FIGS. 15A and 15B.

As shown in FIG. 15A, the main strap 1400 of the automatic shielding structure shields the protective cover 1411 with electrical or mechanical force, and may further include the second processor 1001 and the automatic shielding device 1420.

When the main strap 1400 receives the activation signal generated from the wearable sensor, the second processor 1001 controls the camera 1005 to be activated, and generates and transmits at least one control signal for each of the automatic shielding device 1420 and the camera 1005.

The control signal of the camera 1005 may include a photographing signal and first time information. The camera photographing may be started according to the photographing signal, and the camera photographing may be maintained for a first time corresponding to the first time information. In addition, in some embodiments, the second processor 1001 may check the opening and closing state of the camera 1005 before generating the control signal, and generate the control signal according to the opening and closing state.

The control signal of the automatic shielding device 1420 may include an opening signal, a closing signal, and second time information. The shielding device operates so that the protective cover 1411 is shielded according to the opening signal and the closing signal, and the second time information may mean a second time during a period in which the protective cover 1411 is open between the opening signal and the closing signal.

Meanwhile, the first time and the second time may be different depending on the type of the medication object, and the time length of the first time and the second time may also be different. That is, as shown in 1501 of FIG. 15B, the first time may be shorter than the second time, as shown in 1502 of FIG. 15B, the first time may be longer than the second time, or as shown in 1503 of FIG. 15B, the first time and the second time may have the same time length.

In addition, the start and end times of the first time and the second time may be different. That is, as shown in 1501 of FIG. 15B, the start time of the first time may be later than the start time of the second time, and the end time of the first time may be faster than the end time of the second time. Similarly, as shown in 1502 of FIG. 15B, the start time of the first start time may be faster than the start time of the second time, and the end time of the first time may be slower than the start time of the second time, or as shown in 1503 of FIG. 15B, the start time and the end time of the first time may be faster than the start time and the end time of the second time.

As described above, the wearable device 20 of the present disclosure may customize various photographing environments according to user needs by setting the length of the first time, which is the photographing time of the camera, and the second time, which is the shielding time of the protective cover, and the start and end times differently.

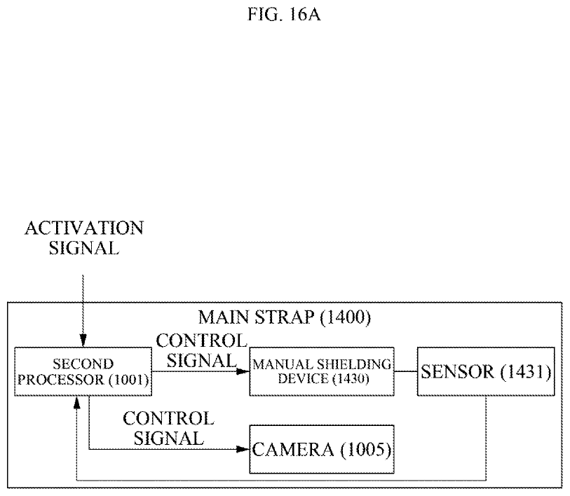

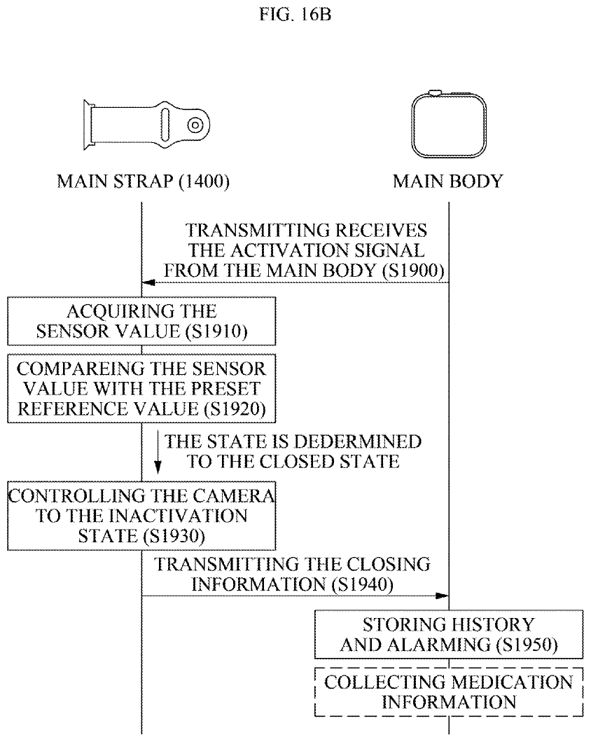

FIG. 16A is a diagram illustrating a structure of a passive shielding protective cover in some embodiments of the present disclosure. In FIG. 16A, a main strap 1400 having a passive shielding structure that shields the protective cover by applying an external force of a user is illustrated.

In the passive shielding structure, the main strap 1400 may further include a second processor 1001, a passive shielding device 1430 including the protective cover 1411, and a sensor 1431 that senses an opening and closing state of the protective cover. Similar to the automatic shielding structure, when the main strap 1400 receives an activation signal generated from a wearable sensor, the second processor 1001 activates the camera 1005.

Thereafter, the second processor 1001 generates a control signal and transmits the control signal to the camera 1005. The control signal of the camera 1005 may include a photographing signal and first time information is the same as the above-described automatic shielding structure. The camera photographing may be started according to the photographing signal, and the camera photographing may be maintained for a first time corresponding to the first time information.

The sensor 1431 senses the shielding state of the protective cover 1411, and generates an opening signal or a closing signal according to the sensing result and transmits the opening signal or the closing signal to the second processor 1001. In the case of the passive shielding structure, unlike the automatic shielding structure, the second processor 1001 does not provide the control signal to the shielding device, so the camera photographing and the protective cover 1411 may not be simultaneously opened. In other words, in the passive shielding structure, whether the protective cover 1411 is shielded by the external force may be determined regardless of whether the camera is photographed or not, so various embodiments may occur according to the gap between the opening time of the protective cover and the camera photographing time.

FIG. 16B is a diagram illustrating the operation of the wearable device 20 according to the closed state of the passive shielding protective cover in some embodiments of the present disclosure. As illustrated in FIG. 16B, when the main strap 1400 receives the activation signal from the main body (S1900), the main strap 1400 acquires the sensor value through the sensor 1431 (S1910), compares the sensor value with the preset reference value to determine the opening and closing state of the protective cover 1411 (S1920).

When the opening and closing state of the protective cover 1411 is determined to be the open state, the main strap 1400 controls the camera to the activation state, and when the opening and closing state of the protective cover 1411 is determined to be the closed state, the main strap 1400 controls the camera to the inactivation state (S1930), and transmits the closing information to the main body (S1940). The main body may store the medication history based on the closing information, and provide a notification for guiding the user that the protective cover 1411 is in the closed state through the display (S1950). Here, the medication history may be a graph related to the medication by time zone and recording information indieating that the medication operation has not been taken.

On the other hand, in various embodiments, the wearable device 20 may generate the control signal according to the input at arbitrary time points of the user regardless of the opening and closing state of the protective cover 1411, control the camera to the activation state, and perform the medication operation photography.

As described above, the wearable device 20 of the present disclosure may collect the medication information of the user individual using the wearable device itself and may collect the medication information using the wearable device itself and may generate a database.

In another embodiment, even though the opening and closing state of the protective cover 1411 is determined to be in the closed state, the wearable device 20 may control the camera 1005 to be in the activated state and transmit a control signal to perform the photographing of the medication action. However, in this case, even though the photographing of the camera is being performed, a section in which the photographing of the medication action of the user is partially missed may occur according to the closing of the protective cover 1411. Hereinafter, the operation of the wearable device 20 when the user opens the protective cover 1411 in the middle will be described in some embodiments.

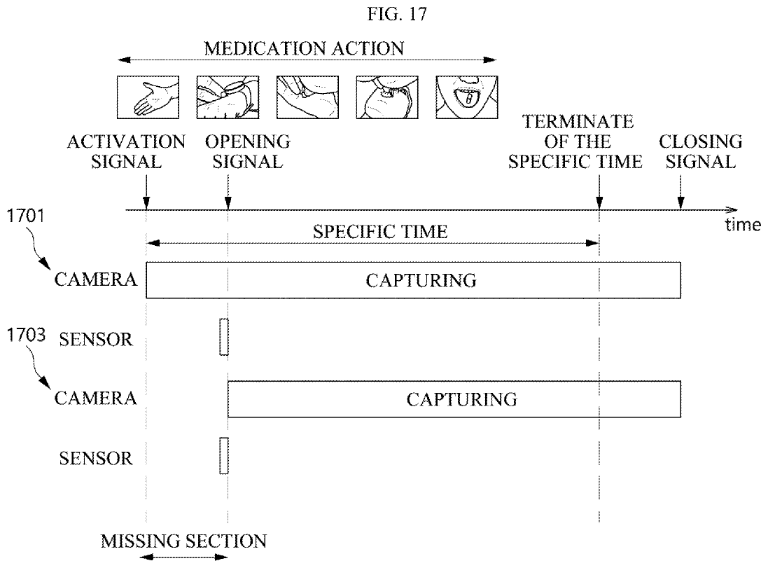

FIG. 17 is a diagram illustrating the operation of the wearable device 20 according to the middle opening of the manually shielded protective cover in some embodiments of the present disclosure. As shown in FIG. 17, when the second processor 1001 of the main strap 1400 receives the activation signal according to the sensing of the medication action of the user, the camera is activated for the first time. In this case, the first time may be a sufficiently long time for the user to complete the medication action, and the first time may be different depending on the type of the medication target as described above.

When the first time is terminated, the second processor 1001 controls the camera to be deactivated. Before the first time is terminated, when the user performs the opening operation of the protective cover 1411 by the external force, the sensor 1431 senses this and transmits the opening signal.

In various embodiments, referring to 1701 of FIG. 17, the second processor 1001 may generate a control signal immediately receiving the activation signal to photograph the camera. In this case, regardless of whether the protective cover 1411 is opened, the photographing of the camera may be performed first, so that a missing section 1710 may occur. That is, the missing section means a section in which the medication action of the user is not photographed due to the closing of the protective cover 1411, and the second processor 1001 may give an additional time by a time corresponding to the missing section to extend the photographing time of the camera.

In various embodiments, referring to 1703 of FIG. 17, the second processor 1001 may generate a control signal from the time when the opening signal is received from the sensor 1431 and capture the camera after receiving the activation signal. In this case, similarly, since the capture starts after the taking operation of the user starts, the missing section 1720 may occur.

In addition, in various embodiments, when the protective cover 1411 is opened by more than a predetermined time (e.g., 20 seconds) from the time when the activation signal is received, the taking operation of the user may have already been terminated, and thus the second processor 1001 may not generate the control signal and may not perform the camera capture.

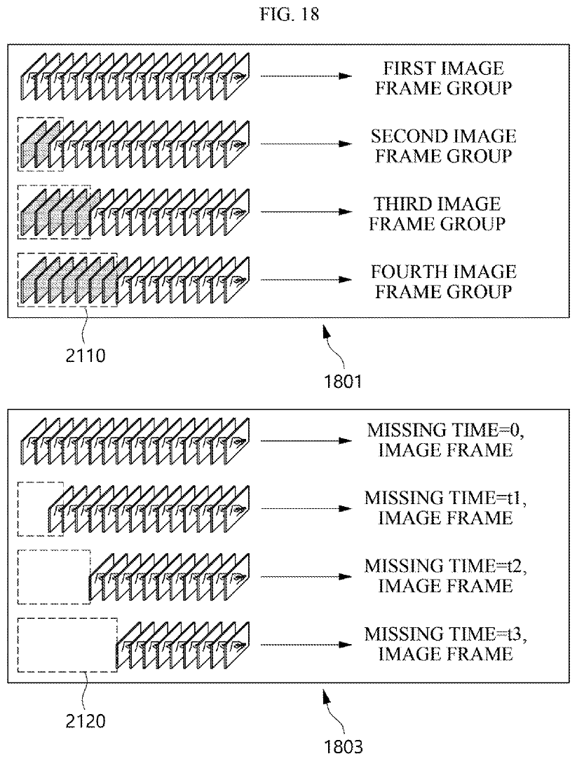

FIG. 18 is a diagram illustrating an AI learning method of the missing section according to the middle opening in some embodiments of the present disclosure. The AI model may be trained through a training data set consisting of a group of taking images for each section for the missing section.

Referring to 1801 of FIG. 18, when the second processor 1001 generates a control signal immediately after receiving the activation signal and captures the camera, the blank image is captured by the closed protective cover 1411 during the missing section, and thus the server 10 may train the AI model by inputting the training data set including the blank image 2110 during the time corresponding to the missing section in the group of taking images for each section.

Referring to 1803 of FIG. 18, when the second processor 1001 generates a control signal from the time when the opening signal is received from the sensor 1431 and captures the camera after receiving the activation signal, the capture is not performed during the missing section, and thus the server 10 may train the AI model by inputting the training data set including no image 2120 during the time corresponding to the missing section in the group of taking images for each section.

Here, as shown in FIG. 18, the second processor 1001 learns the AI model using the medication image frame group that variously sets the time corresponding to the missing section, thereby improving the accuracy of medication determination even when the missing section occurs during camera photographing.

Meanwhile, in various embodiments, the server 10 may determine the medication operation of the user using the AI model, and may calculate the reliability of the medication determination result, and guide the user to directly record the medication if the reliability is low through a nutzi alarm.

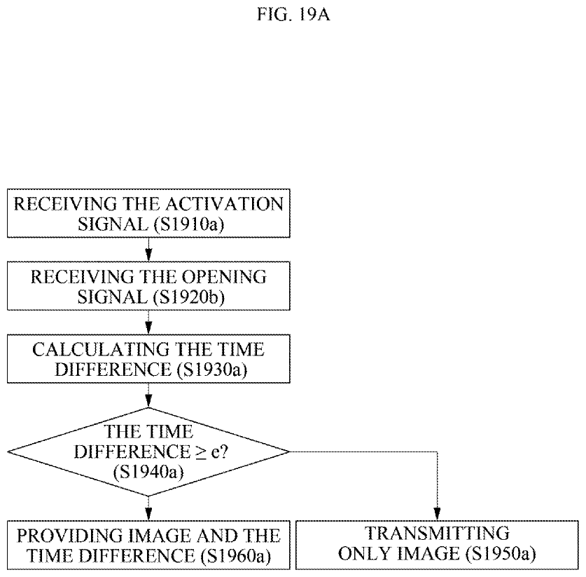

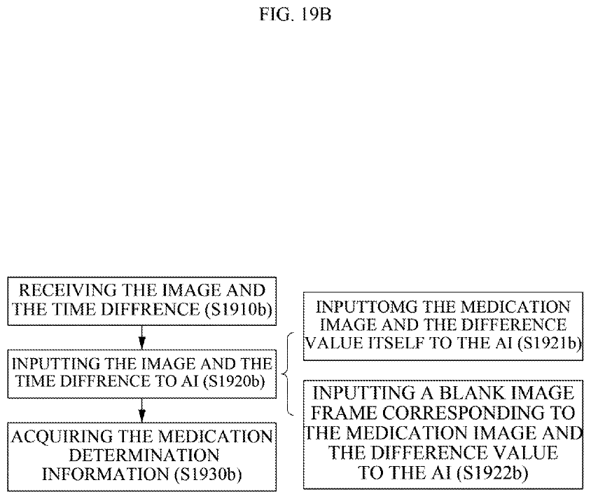

FIG. 19A is a diagram illustrating the operation flow of the wearable device 20 according to the middle opening of the manual shielding protective cover in some embodiments of the present disclosure, and FIG. 19B is a diagram illustrating the operation flow of the server according to the middle opening of the manual shielding protective cover in some embodiments of the present disclosure. Hereinafter, the operations of the wearable device 20 and the server 10 will be described in detail with reference to FIGS. 19A to 19B.

Referring to FIG. 19A, the main strap 1400 of the wearable device 20 receives the activation signal (S1910a), receives the opening signal from the sensor 1431 (S1920a), and calculates the time difference of the time received each signal (S1930a).

Thereafter, the main strap 1400 compares the calculated time difference with the preset time (S1940a), transmits only the photographed medication image to the server 10 if the calculated difference is smaller than the preset time (S1950a), and transmits the photographed medication image to the server 10 along with the difference if the calculated difference is greater than or equal to the preset time (S1960a).

Referring to FIG. 19B, when the server 10 receives the medication image and the difference value (S1910b), information about the medication image and the difference value is input to the AI model (S1920b). In this case, the server 10 may input the medication image and the difference value itself to the AI model (S1921b), or may input a blank image frame corresponding to the medication image and the difference value to the AI model (S1922b). S1922b. Thereafter, the server 10 may acquire the medication determination information as the output of the AI model (S1930b).

### 3. Add-on Wearable Device

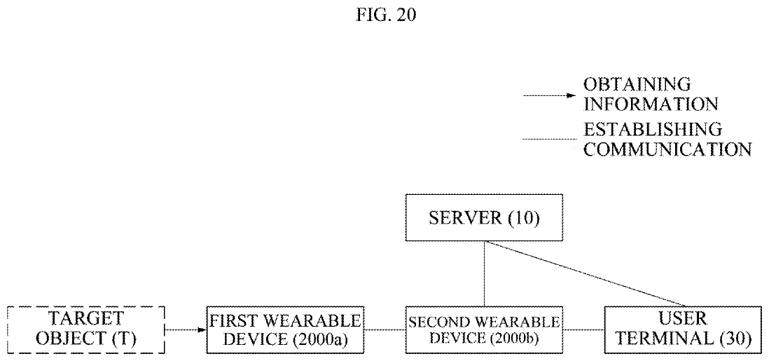

FIG. 20 is a block diagram illustrating an example of a health management system 2000 further including an add-on wearable device (e.g., first wearable device 2000a), according to various embodiments.

According to various embodiments, referring to FIG. 20, the health management system 2000 may include a first wearable device 2000a, a second wearable device 2000b operatively connected to the first wearable device 2000a, a user terminal 30, and a wireless communication device 40 disposed (or provided) in a form associated with a target object T associated with a health management behavior (e.g., attached, coupled, etc.). The health management system 2000 shown in FIG. 20 may be implemented as the health management system 1 described with reference to FIG. 1, and thus redundant descriptions will be omitted. The first wearable device 2000a may be defined as a sub device and the second wearable device 2000b may be defined as a main device, and the sub device may support a service (e.g., a medication management service) for a health management behavior in a state of being physically coupled to the main device.

According to various embodiments, each of the first wearable device 2000a and the second wearable device 2000b may be worn by a user and may be implemented to acquire and/or analyze information about taking a medication based on collaboration. For example, each of the first wearable device 2000a and the second wearable device 2000b may be implemented to perform different functions, thereby enabling collaboration between the first wearable device 2000a and the second wearable device 2000b to be performed. In an embodiment, the first wearable device 2000a may perform a function to capture sequential medicine performance operations according to the performance of the taking action of the user, and the second wearable device 2000b may perform a function to transmit the captured taking a medication performance image 600 to an external device (e.g., the server 10, and/or the user terminal 30). In another embodiment, the second wearable device 2000b may be implemented to acquire other biometric information associated with the taking a medication performance, thereby enabling a service based on the association relationship between the taking a medication performance and the other biometric information (e.g., life log), which will be described below in “5.2. Life log association”. In another embodiment, the second wearable device 2000b may perform an operation to assist the capturing operation of the first wearable device 2000a, which will be described below in “4.1 vision activation”. As collaboration between the first wearable device 2000a and the second wearable device 2000b performing the different functions is performed, the operation burden is reduced for each device, and if appropriately necessary, a health management service (e.g., a taking a medication performance determination service) may be performed, thereby improving convenience of the user.

According to various embodiments, the first wearable device 2000a and the second wearable device 2000b may be provided to be operatively connected to each other. For example, the operational connection may mean at least one of a physical connection (or a combination) or a communication connection for exchanging data.

Meanwhile, the first wearable device 2000a may perform an operation similar to the operation of exchanging data by setting a communication connection directly with the user terminal 30 and cooperating with the second wearable device 2000b described below together with the user terminal 30, without being limited to the illustrated and/or described examples.

### 3.1. Components of an Add-on Wearable Device

According to various embodiments, the add-on wearable device (e.g., the first wearable device 200a) may be implemented in the form of a strap fastened to the main body of the smart watch. The main body of the smart watch is an electronic product produced by a third party (e.g., another smart watch manufacturer), and the add-on wearable device (e.g., the first wearable device 200a) according to various embodiments may be implemented to cooperate with the electronic product of the other third party to provide a medication management service.

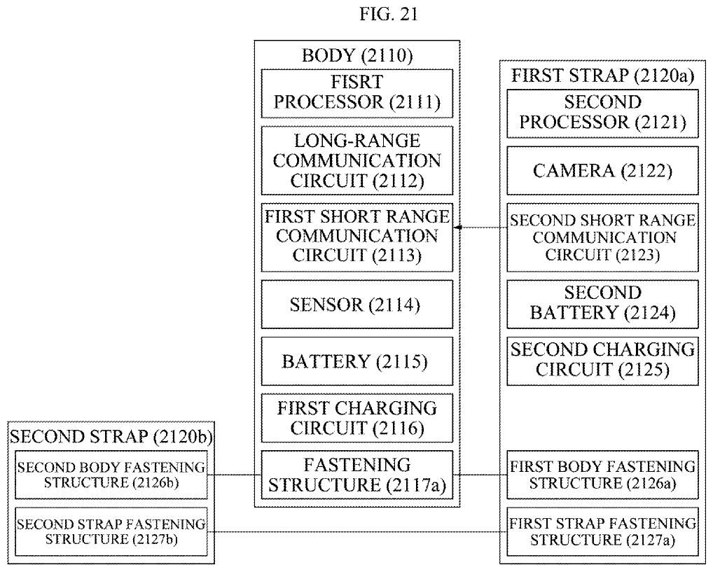

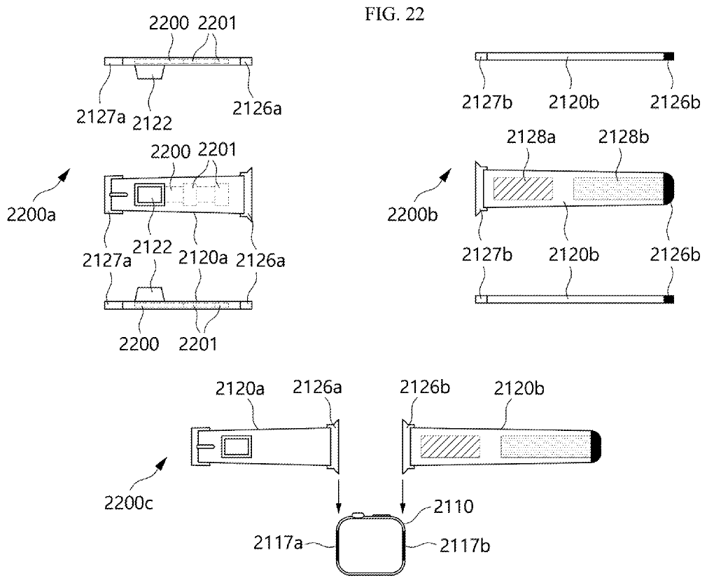

FIG. 21 is a block diagram for describing an example of a configuration of the first wearable device 200a and the second wearable device 200b according to various embodiments. Hereinafter, FIG. 21 will be further described with reference to FIG. 22.

FIG. 22 is a diagram for describing an example of the first wearable device 200a and the second wearable device 200b according to various embodiments.

According to various embodiments, referring to FIGS. 21 to 22, the first wearable device 2000a may be implemented as at least one strap (e.g., first strap 2120a and second strap 2120b), which is physically detachable from the main body (e.g., body 2110) of the smart watch. The at least one strap (e.g., first strap 2120a and second strap 2120b) may be formed to be physically detachable from the body 2111, but not include a component directly electrically connected to the body 2111, and may be implemented to be collaborative, thereby improving convenience in use. The first strap 2120a may function as a main strap for capturing a medication performance image of the user and transmitting the captured medication performance image to the body 2110, and the second strap 2120b may be a sub strap, and the degree of fastening to the first strap 2120a may be adjusted, thereby adjusting the degree of tightening by the first strap 2120a and the second strap 2120b to a part (e.g., wrist) of the user's body.

According to various embodiments, referring to FIG. 21, the first strap 2120a may include a first processor 2121, a camera 2122, a first short-range communication circuit 2123, a first battery 2124, a first charging circuit 2125, a first body fastening structure 2126a, and a first strap fastening structure 2127a, and the second strap 2120b may include a second body fastening structure 2126b and a second strap fastening structure 2127b. However, the at least one strap (e.g., first strap 2120a and 2120b) is not limited to the illustrated and/or described examples, and may be implemented to include more or fewer configurations. Some electronic components (e.g., first processor 2121, camera 2122, and first short-range communication circuit 2123) of the first strap 2120a may be implemented like the wearable device 20 described above, and thus redundant descriptions will be omitted.

According to various embodiments, referring to FIG. 22, the electronic components 2200 and 2201 (e.g., the first processor 2121, the camera 2122, the first short-range communication circuit 2123, the battery 2124, and the first charging circuit 2125) of the first strap 2120a may be disposed on a flexible substrate (e.g., an electronic circuit, an electrical circuit) 2200 (e.g., a flexible printed circuit board (FPCB)) disposed inside the first strap 2120a and electrically connected to each other. In this case, a terminal of the camera 2122 formed on the outside may be disposed on the flexible substrate 2200.

According to various embodiments, the first short-range communication circuit 2123 may be implemented to establish a short-range communication connection with an external device. The short-range communication may be a communication connection based on Bluetooth, BLE, Wi-Fi direct, and/or near field communication (NFC). The first short-range communication circuit 2123 may be implemented to support a function of establishing a short-range communication connection with each of a plurality of external devices. For example, the first short-range communication circuit 2123 may be implemented to establish a first short-range communication connection with the body 2111 (e.g., the second short-range communication circuit 2113), and to establish a second short-range communication connection with the wireless communication device 40 attached to the target object T. Accordingly, the first strap 2120a (e.g., the first processor 2121) may activate the camera 2122 based on an activation signal received from the wireless communication device 40, and transmit a medicine execution image captured using the activated camera 2122 to the body 2110. Meanwhile, without limitation to the described example, the first short-range communication circuit 2123 may be implemented to establish a communication connection directly with the user terminal 30.

According to various embodiments, referring to FIG. 22, the camera 2122 may be provided inside a camera housing formed to be drawn in a vertical direction of the first strap 2120a. The camera 2122 may be disposed inside the camera housing so as to have a photographing direction in the direction of the user's hand when the first strap 2120a is worn by the user.

According to various embodiments, the first battery 2124 may be implemented to store power for providing to the components (e.g., the first processor 2121, the camera 2122, and the first short-range communication circuit 2123) of the first strap 2120a.

According to various embodiments, the first charging circuit 2125 may be implemented to charge the first battery 2124. However, it is not limited to the illustrated and/or described examples, and the first battery 2124 may be replaced without the first charging circuit 2125.

According to various embodiments, the body fastening structure (e.g., the first body fastening structure 2126a and the second body fastening structure 2126b) may be implemented to be physically detached from the body 2110 (or main body) of the smart watch. Referring to FIG. 22, the body fastening structure (e.g., the first body fastening structure 2126a and the second body fastening structure 2126b) may be implemented on one end of the strap (e.g., the first strap 2120a and the second strap 2120b). The body fastening structure (e.g., the first body fastening structure 2126a and the second body fastening structure 2126b) may be implemented to be detachable from one end of the strap (e.g., the first strap 2120a and the second strap 2120b), and may be implemented in various types corresponding to the type of the body 2110 of the smart watch. For example, a size of the body fastening structure (e.g., the first body fastening structure 2126a and the second body fastening structure 2126b) may correspond to the type of the smart watch and/or the size of the body. Based on the specific type of the body fastening structure (e.g., the first body fastening structure 2126a and the second body fastening structure 2126b), the strap (e.g., the first strap 2120a and the second strap 2120b) may be connected to the body 2110 of the smart watch used by the user.

According to various embodiments, the strap fastening structure (e.g., the first strap fastening structure 2127a, the second strap fastening structure 2127b) may be formed at the other end of the strap (e.g., the first strap 2120a, the second strap 2120b), and the two straps (e.g., the first strap 2120a, the second strap 2120b) may be fastened to each other. For example, as the one strap fastening structure (e.g., the first strap fastening structure 2127a) is formed in a structure including a hole as shown in FIG. 22, the two straps (e.g., the first strap 2120a, the second strap 2120b) may be fastened to each other by inserting another strap fastening structure (e.g., the second strap fastening structure 2127b) into the hole, but the present disclosure is not limited thereto. In this case, referring to FIG. 22, the one strap (e.g., the second strap 2120b) may further include at least one length adjusting member 2128a, 2128b. For example, the adhesive members (e.g., the nonwoven fabric) 2128a, 2128b that are detachable from each other may be formed on the outer surface of the second strap 2120b, and the second strap 2120b may be bent so that the two adhesive members (e.g., the nonwoven fabric) 2128a, 2128b come into contact while the other end (e.g., the second strap fastening structure 2127b) of the second strap 2120b penetrates the hole (e.g., the first strap fastening structure 2127a) of the first strap 2120a and is fastened. As the position of the two adhesive members (e.g., the nonwoven fabric) 2128a, 2128b come into contact may be changed, the length of the other end (e.g., the second strap fastening structure 2127b) of the second strap 2120b penetrating the hole (e.g., the first strap fastening structure 2127a) of the first strap 2120a may be adjusted, and as a result, the degree of tightening may be adjusted by the user's body part (e.g., the wrist) by the two straps (e.g., the first strap 2120a, the second strap 2120b).

According to various embodiments, referring to FIGS. 21 to 22, the second wearable device 2000b is a main body (e.g., body 2110) of a smart watch and may be implemented to be physically detached from the at least one strap (e.g., first strap 2120 a, second strap 2120 b) described above. The body 2110 is an electronic product produced by a third party (e.g., another smart watch manufacturer), and may be implemented to provide a medication management service in cooperation with the at least one strap (e.g., first strap 2120 a, second strap 2120 b) described above, but is not limited to the examples described, and may be manufactured by a manufacturer of at least one strap (e.g., first strap 2120 a, second strap 2120 b) rather than the third party.

According to various embodiments, referring to FIG. 21, the body 2110 may include a second processor 2111, a long-range communication circuit 2112, a second short-range communication circuit 2113, a sensor 2114, a second battery 2115, a first charging circuit 2116, and a fastening structure 2117. However, the at least one strap (e.g., first strap 2120 a, second strap 2120 b) is not limited to the illustrated and/or described examples, and may be implemented to include more or fewer configurations. Some electronic components (e.g., second processor 2111, long-range communication circuit 2112, second short-range communication circuit 2113, sensor 2114, second battery 2115 and first charging circuit 2116) of the body 2110 may be implemented like the wearable device 20 described above, and thus redundant descriptions are omitted.

According to various embodiments, the long-range communication circuit 2112 may be implemented to establish a long-range communication connection with the server 10 and/or the user terminal 30. The long-range communication may include wireless LAN communication such as Wi-Fi, and/or cellular communication. The body 2110 (e.g., the second processor 2111) may transmit the received medication performance image 600 to the server 10 and/or the user terminal 30 through the long-range communication circuit 2112. According to various embodiments, the second short-range communication circuit 2113 may be implemented to establish a short-range communication connection with the first strap 2120a. The body 2110 (e.g., the second processor 2111) may receive the medication performance image 600 captured by the first strap 2120a through the short-range communication circuit 2113, and/or transmit a control signal to the first strap 2120a.

According to various embodiments, the body 2110 may be implemented to be connected to the at least one strap (e.g., the first strap 2120a and the second strap 2120b) based on the fastening structure 2117.

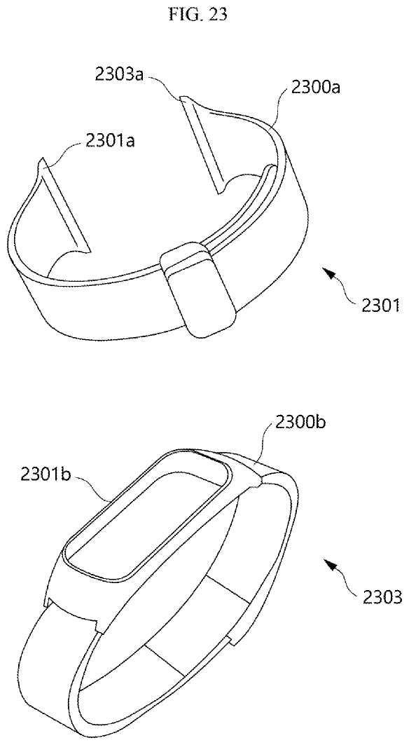

FIG. 23 is a diagram for explaining an example implemented in a single strap form of the first wearable device 2000a according to various embodiments.

Meanwhile, referring to FIG. 23, according to various embodiments, the first strap 2120a may be implemented as a single strap rather than the plurality of straps (e.g., the first strap 2120a and the second strap 2120b), and may be fastened to the body 2110. As shown in 2301 of FIG. 23, the first strap 2120a may be implemented as a single strap 2300a including a plurality of body fastening structures (2301a, 2303a) to be coupled to a fastening structure formed on both sides of the body 2110, and/or as shown in 2303 of FIG. 23, the first strap 2120a may be implemented as a single strap 2300b including a single body fastening structure (2301b) to be coupled to surround the side surface of the body 2110, but may be implemented as a single strap of various shapes not limited to the illustrated and/or described examples. The components disposed inside the single straps 2300a and 2300b are the same as the first strap 2120a, and thus detailed descriptions will be omitted.

### 3.1.2. Additional Examples of Add-on Wearable Devices

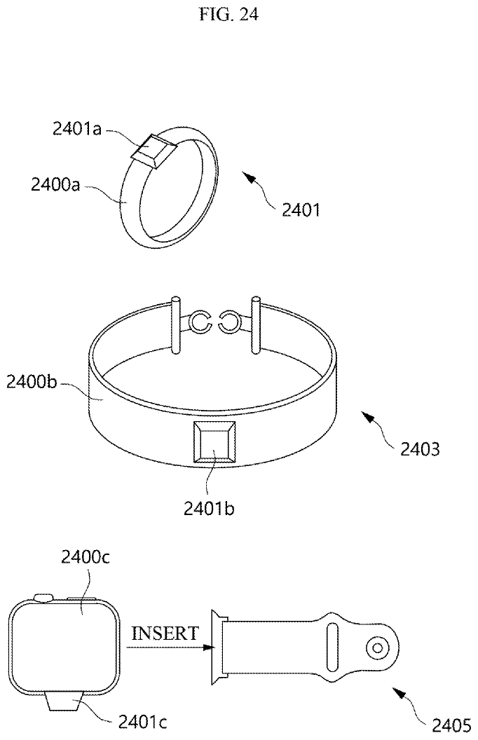

FIG. 24 is a diagram illustrating various examples of an add-on wearable device (e.g., the first wearable device 2000a) according to various embodiments.

In an embodiment, referring to 2401 of FIG. 24, the first wearable device 2000a is implemented as a wearable ring 2400a that can be worn on the user's finger, and the wearable ring 2400a may include a camera 2401a for obtaining the medication performance image 600. The configuration of the wearable ring 2400a may be implemented similarly to the first strap 2120a described above, and thus redundant descriptions will be omitted.

In an embodiment, referring to 2403 of FIG. 24, the first wearable device 2000a is implemented as a bracelet 2400b that can be worn on the user's wrist, and the bracelet 2400b may include a camera 2401b for obtaining the medication performance image 600. The configuration of the wearable ring 2400a may be implemented similarly to the first strap 2120a described above, and thus redundant descriptions will be omitted.

In an embodiment, referring to 2405 of FIG. 24, the first wearable device 2000a is implemented as an additional member 2400c (e.g., a bone dance) that can be detached from the strap of the smart watch, and the additional member 2400c may include a camera 2401c for obtaining the medication performance image 600. The configuration of the additional member 2400c may be implemented similarly to the first strap 2120a described above, and thus redundant descriptions will be omitted.

Hereinafter, for convenience of description, an example in which the first wearable device 2000a is implemented in the form of a strap will be described.

### 3.2. Operation Method of Health Management System 2000

### 3.2.1. Basic Operation of Health Management System 2000

Hereinafter, an example of a basic operation of the health management system 2000 further including an add-on wearable device (e.g., the first wearable device 200a) according to various embodiments will be described. The description overlapping with the basic operation of the health management system I described with reference to FIG. 13 will be omitted.

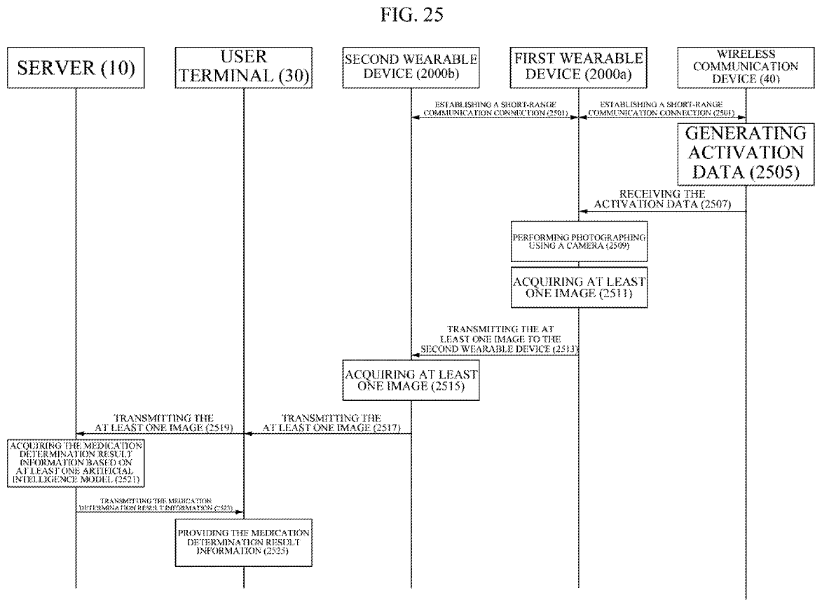

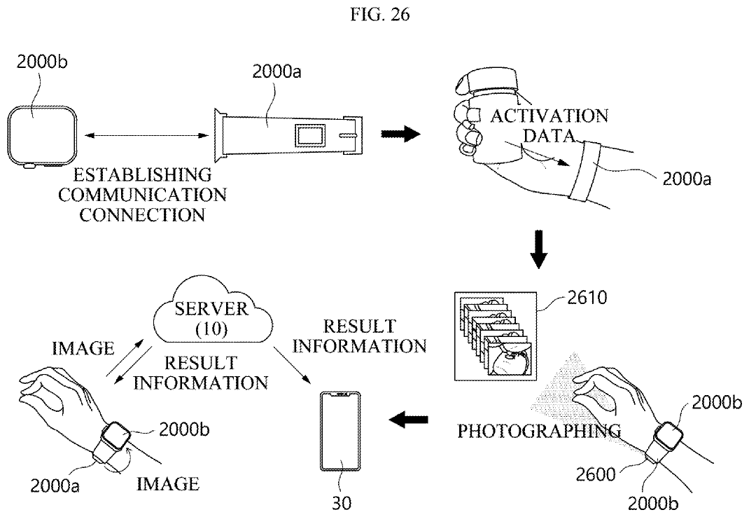

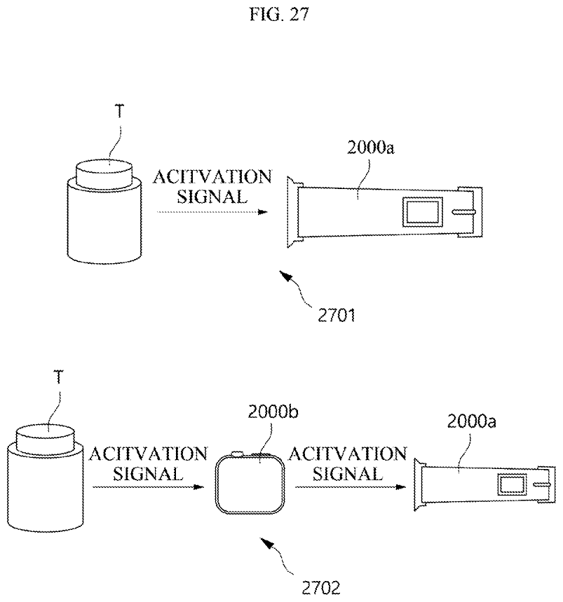

FIG. 25 is a flowchart illustrating a basic operation of the health management system 2000 further including an add-on wearable device (e.g., the first wearable device 200a) according to various embodiments. Operations may be performed regardless of the order of the operations shown and/or described, and more operations may be performed and/or fewer operations may be performed. Hereinafter, FIG. 25 will be further described with reference to FIGS. 26 to 27.

FIG. 26 is a diagram illustrating an example of providing a health management service for performing a medication based on the first wearable device 200a according to various embodiments. FIG. 27 is a diagram illustrating an example of a communication connection method of the first wearable device 200a according to various embodiments.

According to various embodiments, the first wearable device 2000a may establish a short-range communication connection based on the second wearable device 2000b in operation 2501, and establish a short-range communication connection with the wireless communication device 40 in operation 2503. For example, as illustrated in FIG. 26, the first wearable device 2000a (e.g., strap) may receive a request for a communication connection from the second wearable device 2000b (e.g., the main body of the smart watch) physically coupled to the second short-range communication circuit 2123 and establish a communication connection with the second wearable device 2000b. For example, as illustrated in 2701 or 2702 of FIG. 27, the first wearable device 2000a (e.g., strap) may be implemented to acquire an activation signal broadcast from the wireless communication device 40. The first wearable device 2000a (e.g., strap) may acquire an activation signal broadcast from the wireless communication device 40 based on identification information of the wireless communication device 40 received from the second wearable device 2000b based on the short-range communication connection. For example, the first wearable device 2000a may be implemented to establish a communication connection with the wireless communication device 40 attached to the target object T based on the identification information of the wireless communication device 40 or to scan a signal (e.g., an advertisement signal) including activation data broadcast from the wireless communication device 40 based on the identification information of the wireless communication device 40 without establishing the communication connection.

According to various embodiments, the wireless communication device 40 may generate activation data in operation 2505. According to various embodiments, the first wearable device 2000a may receive the activation data in operation 2507, perform photographing using a camera in operation 2509, acquire at least one image in operation 2511, and transmit the at least one image to the second wearable device 2000b in operation 2513. For example, referring to FIG. 26, as the movement of the target object T occurs according to the taking performance of the user, the wireless communication device 40 may generate and transmit activation data to the first wearable device 2000a. At this time, the first wearable device 2000a may scan the activation data included in the advertisement signal broadcast from the wireless communication device 40 irrespective of the communication connection setting or the activation data transmitted based on the short-range communication connection setting as described above. The first wearable device 2000a may acquire the medication image 2610 by activating the camera 2600 based on the activation data. At this time, instead of transmitting the medication image to the second wearable device 2000b as described above, the first wearable device 2000a may transmit the medication image to the user terminal 30 based on the direct communication connection setting.

According to various embodiments, the second wearable device 2000b may acquire at least one image (e.g., the medication image 2610) in operation 2515, and transmit the at least one image (e.g., the medication image 2610) to the server 10 in operation 2517. At this time, the transmission of the (e.g., the medication image 2610) to the server 10 may be performed via the user terminal 30 in operation 2519, but is not limited to the described example.

According to various embodiments, the server 10 may acquire the medication determination result information based on at least one artificial intelligence model (e.g., at least one of the classification model 610, the detection model 620, or the confirmation model 630) and at least one image in operation 2521, and transmit the medication determination result information to the user terminal 30 in operation 2523. According to various embodiments, the user terminal 30 may provide the medication determination result information in operation 2523.

### 3.2.1.1. Registration of an Add-on Wearable Device

According to various embodiments, information about the user's add-on wearable device (e.g., the first wearable device 200a) may be registered in the server 10 so that the medication management service may be performed.

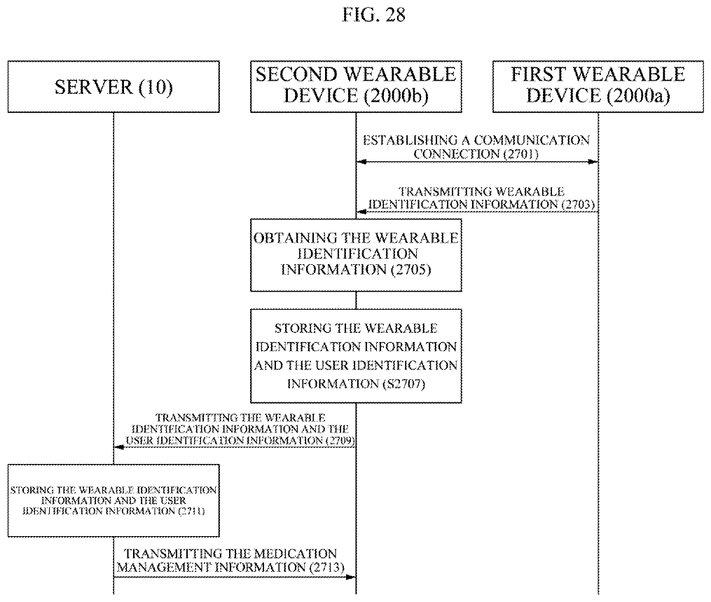

FIG. 28 is a flowchart illustrating an example of a communication connection setting operation of the first wearable device 200a and the second wearable device 200b according to various embodiments. Operations may be performed regardless of the order of the operations shown and/or described, and more operations may be performed and/or fewer operations may be performed.

According to various embodiments, the first wearable device 200a may establish a communication connection with the second wearable device 200b in operation 2701 and transmit wearable identification information to the second wearable device 200b in operation 2703.

According to various embodiments, the second wearable device 200b may obtain the wearable identification information in operation 2705, store the wearable identification information and the user identification information (e.g., the user ID) in a related form in operation 2707, transmit the wearable identification information and the user identification information (e.g., the user ID) to the server 10 in operation 2709. According to various embodiments, the server 10 may store the wearable identification information and the user identification information (e.g., the user ID) in a related form in operation 2711, and transmit the medication management information in operation 2713. The server 10 may register the wearable identification information and the user identification information, and then store and manage health management data in a related form to the wearable identification information and the user identification information. Subsequently, when a request for health management data is received from the user terminal 30 and/or the second wearable device 200b, the stored health management data corresponding to the wearable identification information or the user identification information may be provided to the user terminal 30 and/or the second wearable device 2000b.

### 3.2.1.2. Remote Control of an Add-on Wearable Device.

According to various embodiments, the add-on wearable device (e.g., the first wearable device 200a) may be controlled by another external device (e.g., the user terminal 30, or the second wearable device 200b). Accordingly, the add-on wearable device (e.g., the first wearable device 200a) may be implemented so as not to include electronic components for communieating with the user such as a display, a touch screen, etc.

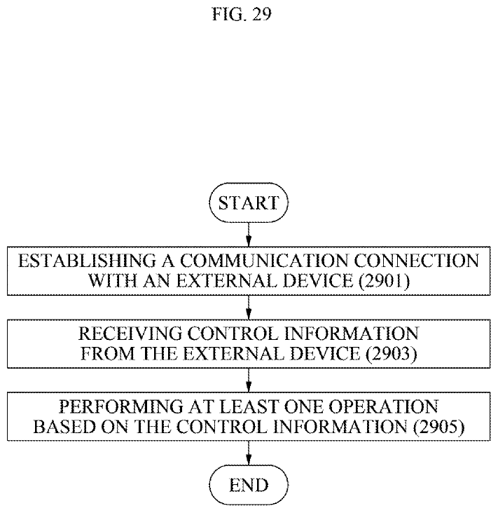

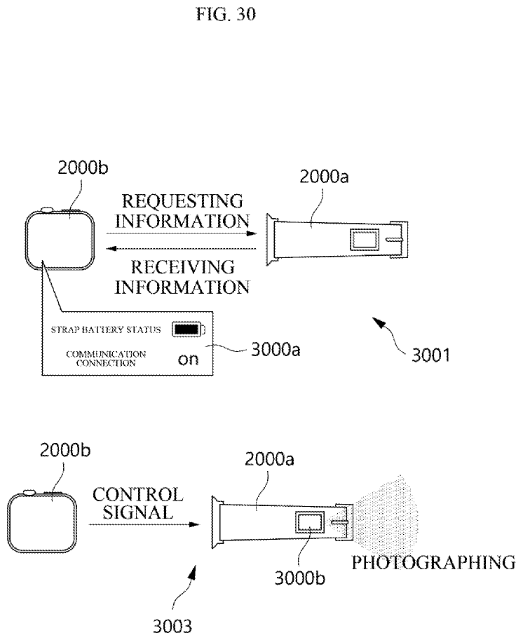

FIG. 29 is a flowchart illustrating a remote control operation of the first wearable device 200a according to various embodiments. Operations may be performed regardless of the order of operations shown and/or described, and more operations may be performed and/or fewer operations may be performed. Hereinafter, FIG. 29 will be further described with reference to FIG. 30.

FIG. 30 is a diagram illustrating an example of remote control of the first wearable device 200a according to various embodiments.

According to various embodiments, the first wearable device 200a may establish a communication connection with an external device (e.g., the second wearable device 200b, and/or the user terminal 30) in operation 2901, and receive control information from the external device (e.g., the second wearable device 200b, and/or the user terminal 30) in operation 2903.

According to various embodiments, in operation 2905, the first wearable device 2000a may perform at least one operation based on the control information. In an embodiment, the first wearable device 2000a may perform an operation of providing information on the first wearable device 2000a to the external device (e.g., the second wearable device 2000b and/or the user terminal 30). For example, as shown in 3001 of FIG. 30, based on the execution of an application (e.g., the first application 900) installed in the second wearable device 2000b, the second wearable device 2000b may provide an execution screen including information 3000a (e.g., battery information, communication connection state information) on the second wearable device 2000b. In this case, the first wearable device 2000b may provide information 3000a (e.g., battery information, communication connection state information) on the second wearable device 2000b to the second wearable device 2000b and provide the information 3000a on the second wearable device 2000b on the execution screen based on the request of the second wearable device 2000b. In another embodiment, the first wearable device 2000a may perform an operation based on a control signal of the external device (e.g., the second wearable device 2000b and/or the user terminal 30). For example, as shown in 3003 of FIG. 30, the first wearable device 2000a may perform photographing using the camera 3000b based on a camera control signal received from the second wearable device 2000b.

### 3.2.2. Mode of an Add-on Wearable Device

According to various embodiments, the add-on wearable device (e.g., the first wearable device 200a) may be implemented to be set to a plurality of modes that perform different functions. Based on the plurality of modes, the amount of power consumption of the battery (e.g., the first battery 2124) may be saved as the operation burden of the add-on wearable device (e.g., the first wearable device 200a) is reduced.

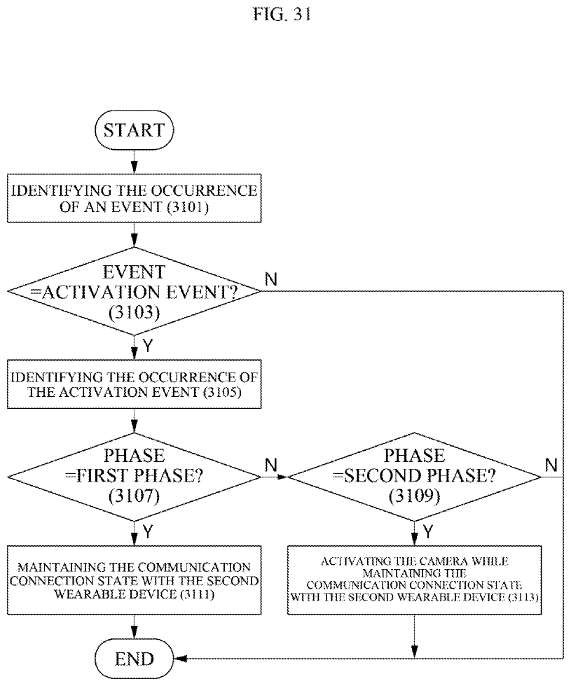

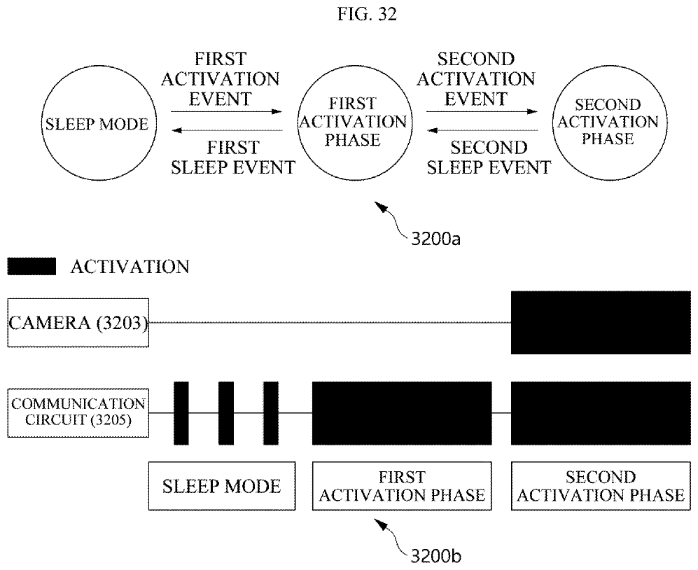

FIG. 31 is a flowchart illustrating an example of operations for each of a plurality of modes of the add-on wearable device (e.g., the first wearable device 200a), according to various embodiments. Operations may be performed regardless of the order of operations shown and/or described, and more operations may be performed and/or fewer operations may be performed. Hereinafter, FIG. 31 will be further described with reference to FIG. 32.

FIG. 32 is a diagram illustrating an example of a plurality of modes of the add-on wearable device (e.g., the first wearable device 200a), according to various embodiments.

According to various embodiments, the first wearable device 2000a may identify the occurrence of an event in operation 3101. The first wearable device 2000a may determine whether the event generated in operation 3103 corresponds to an activation event, and if the event corresponds to the activation event, the first wearable device 2000a may identify the occurrence of the activation event in operation 3105. For example, referring to 3200a of FIG. 32, the mode 3201 of the first wearable device 2000a may include a sleep mode and an activation mode, and the activation mode may include a plurality of activation steps (e.g., a first activation step and a second activation step).

According to various embodiments, the degree of operation of the first wearable device 2000a (or the degree of activation of the electronic components) may be different for each mode of the first wearable device 2000a. In an embodiment, in the activation mode, the degree of operation (or the degree of activation) may be set to be greater than in the sleep mode. For example, referring to 3200b of FIG. 32, when the mode of the first wearable device 2000a is set to the sleep mode, only the communication circuit 3205 of the first wearable device 2000a may be activated at a pre-set first period, and the first wearable device 2000a may set a communication connection with an external device (e.g., the second wearable device 2000b, the user terminal 30, and/or the wireless communication device 40) at the first period for a specific time. Accordingly, the first wearable device 2000a may intermittently exchange data (or information) with an external device (e.g., the second wearable device 2000b, the user terminal 30, and/or the wireless communication device 40) based on the communication connection set for the specific time. For example, referring to 3200b of FIG. 32, when the mode of the first wearable device 2000a is set to the first activation phase, the activation state of the communication circuit 3205 of the first wearable device 2000a may be maintained (or activated at a second period shorter than the first period), and the first wearable device 2000a may continue to maintain a communication connection with an external device (e.g., the second wearable device 2000b, the user terminal 30, and/or the wireless communication device 40). Accordingly, the first wearable device 2000a may be controlled to react in real time to data received from the external device based on the communication setting. For example, referring to (3200b of FIG. 32, when the mode of the first wearable device 2000a is set to the second activation phase, the activation state of the communication circuit 3205 and the camera 3203 of the first wearable device 2000a is maintained, and the first wearable device 2000a may transmit the image captured by the camera 3203 to an external device (e.g., the second wearable device 2000b, the user terminal 30, and/or the wireless communication device 40).

According to various embodiments, the mode of the first wearable device 2000a may be switched based on an event occurring, and when an activation event occurs, the mode of the first wearable device 2000a may be set to a first activation stage or a second activation stage, and when a deactivation event occurs, the mode of the first wearable device 2000a may be set to a sleep mode. For example, the activation event may include a first activation event for switching from the sleep mode to the first activation stage, and the first activation event may occur when an activation signal is received, but is not limited to the described example.

For example, the activation event may include a second activation event for switching from the first activation stage to the second activation stage, and the second activation event may occur when a shielding structure of the camera is opened, but is not limited to the described example.

For example, the activation event may include a first deactivation event for switching from the activation mode (e.g., the first activation stage and the second activation stage) to the sleep mode, and the first deactivation event may occur when a predetermined period elapses from a time when the activation signal is received, but is not limited to the described example.

Meanwhile, the described example is not limited, and an event for switching from the second activation stage to the first activation stage may occur.

According to various embodiments, the first wearable device 2000a may determine whether the phase of the activation event corresponds to the first phase in operation 3107, and determine whether the phase of the activation event corresponds to the second phase in operation 3109 when the phase of the activation event does not correspond to the first phase (3107-N). According to various embodiments, the first wearable device 2000a may activate the camera while maintaining the communication connection state with the second wearable device 2000b in operation 3113 when the activation phase is the first phase (3107-Y), and maintain the communication connection state with the second wearable device 2000b in operation 3111 when the activation phase is the second phase (3111-Y).

### 3.2.3. Inter-Complement Operation

According to various embodiments, when the power of one of the first wearable apparatus 200a and the second wearable apparatus 200b is off, the operation may be performed instead of the other wearable apparatus.

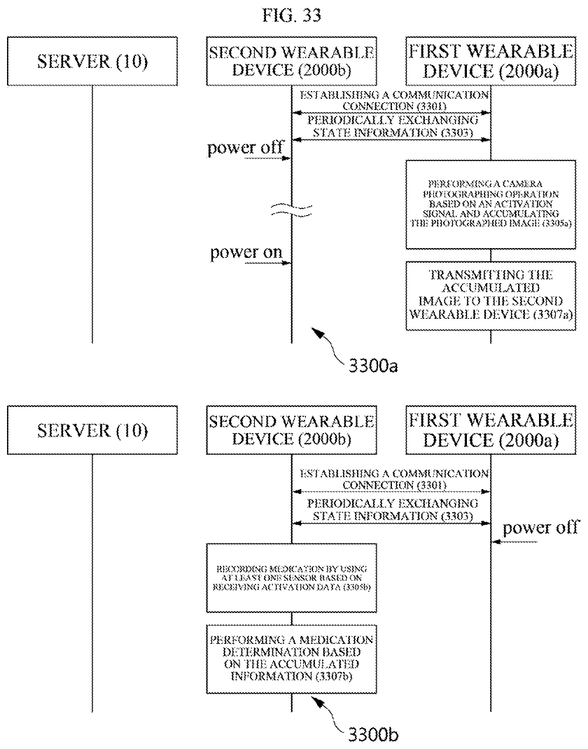

FIG. 33 is a flowchart illustrating an example of an operation of another wearable apparatus when the power of one of the first wearable apparatus 200a and the second wearable apparatus 200b is off according to various embodiments. Operations may be performed regardless of the order of the operations shown and/or described, and more operations may be performed and/or fewer operations may be performed.

According to various embodiments, referring to 3300a of FIG. 33, when the second wearable device 2000b is powered off, the first wearable device 2000a may store and accumulate the medication performance image, and when the second wearable device 2000b is powered on, the accumulated medication performance image may be transmitted to the second wearable device 2000b. The first wearable device 2000a and the second wearable device 2000b may establish a communication connection in operation 3301 and periodically exchange state information in operation 3303. Accordingly, each wearable device (e.g., the first wearable device 2000a and the second wearable device 2000b) may periodically identify the power state of another wearable device based on the state information. When the second wearable device 2000b is identified as being powered off, the first wearable device 2000a may perform a camera photographing operation based on an activation signal and accumulate the photographed image in operation 3305a, and when the second wearable device 2000b is powered on, transmit the accumulated image to the second wearable device 2000b in operation 3307a, so that the medication determination based on the photographed image is performed while the second wearable device 2000b is powered off through the server 10. Meanwhile, the first wearable device 2000a may be implemented to set a communication connection with the server 10 and/or the user terminal 30 directly and transmit the photographed image to the server and/or the user terminal 30 when the second wearable device 2000b is identified as being powered off, without being limited to the illustrated and/or described examples.

According to various embodiments, referring to 3300b of FIG. 33, when the power of the first wearable device 2000a is off, the second wearable device 2000b may acquire information about taking a medication by using at least one sensor included in the second wearable device 2000b. When the power of the first wearable device 2000a is identified as being off, the second wearable device 2000b may record medication by using at least one sensor based on receiving activation data from the wireless communication device 40 (e.g., setting a communication connection with the wireless communication device 40 or scanning a signal broadcast from the wireless communication device 40) in operation 3305b, and perform a medication determination based on the accumulated information in operation 3307b. In other words, when the power of the first wearable device 2000a is identified as being off, the second wearable device 2000b may initiate an operation of acquiring information about taking a medication by using a self-implemented sensor instead of the first wearable device 2000a. An example of an operation of acquiring information about taking a medication by using a self-implemented sensor instead of the first wearable device 2000a will be described in detail in the “4.2. Nonvision Algorithm”.

### 3.2.4. Privacy Protection

According to various embodiments, when the first wearable device 2000a is lost, the lost first wearable device 2000a may be used by another user and personal information may be abused. As described above, the second wearable device 2000b may prevent abuse problems caused by the loss by authentieating the first wearable device 2000a based on the registered wearable identification information and user identification information.

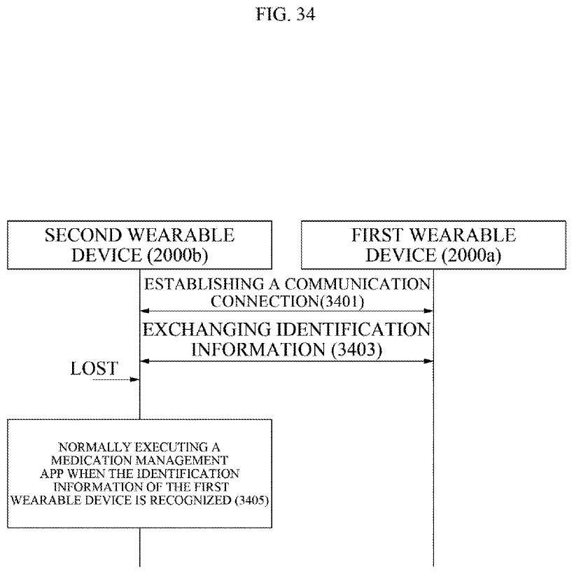

FIG. 34 is a flowchart illustrating an example of an operation of the health management system 2000 when the first wearable device 2000a is lost, according to various embodiments. Operations may be performed regardless of the order of the operations shown and/or described, and more operations may be performed and/or fewer operations may be performed.

According to various embodiments, the first wearable device 2000a and the second wearable device 2000b may establish a communication connection in operation 3401 and exchange identification information (e.g., wearable identification information and user identification information) with each other in operation 3403.

According to various embodiments, the second wearable device 2000b may normally execute a medication management app (e.g., the first application 900) when the identification information of the first wearable device 2000a (e.g., the strap) is recognized in operation 3405. For example, as described above, the second wearable device 2000b (or the server 10 or the user terminal 30) may register (or store) the identification information (e.g., wearable identification information) of the first wearable device 2000a used by the user and the user identification information in a related form. After the registration, when the identification information of the first wearable device 2000a is received, the second wearable device 2000a may be implemented to normally execute the first application 900 to provide the medication management service when the second wearable device 2000a is authenticated based on comparison with the registered information.

### 3.3. Charging Device

### 3.3.1. Components of Charging Device

According to various embodiments, a charging device for charging the above-described wearable devices (e.g., the first wearable device 2000a and the second wearable device 200b) together may be provided.

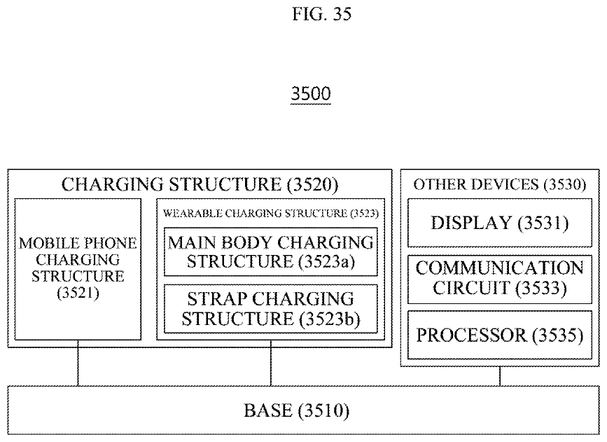

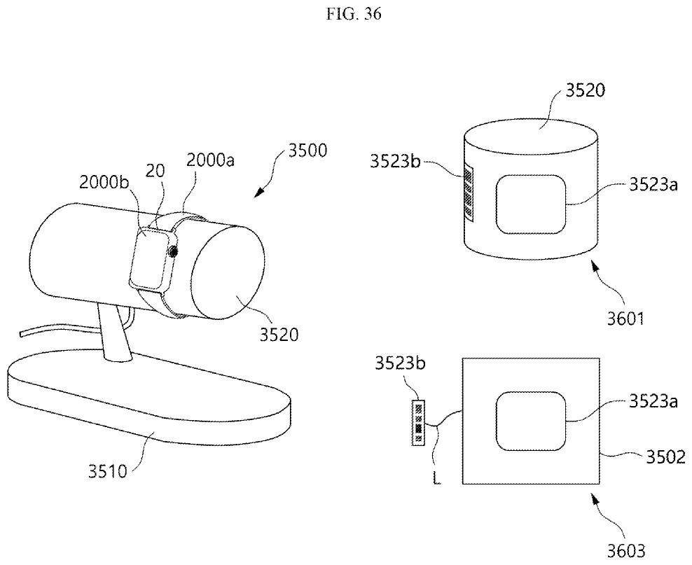

FIG. 35 is a block diagram of a charging device 3500 according to various embodiments. Hereinafter, FIG. 35 will be further described with reference to FIG. 36.

FIG. 36 is a diagram for describing an example of a charging device 3500 according to various embodiments.

According to various embodiments, referring to FIG. 35, the charging device 3500 may include a base 3510, a charging structure 3520 provided on the base 3510, and other devices 3530. However, the charging device 3500 is not limited to the described and/or illustrated examples, and the charging device 3500 may be implemented to include more and/or fewer configurations. According to various embodiments, the charging structure 3520 may include a mobile phone charging structure 3521 for charging the user terminal 30, and a wearable charging structure 3523 for charging the wearable device 20.

According to various embodiments, the wearable charging structure 3523 may include a main body charging structure 3523a and a strap charging structure 3523b to simultaneously charge the first wearable device 2000a and the second wearable device 2000b described above. For example, the strap charging structure 3523b may be implemented to charge the first wearable device 2000a (e.g., the first strap 2120a), and the main body charging structure 3523a may be implemented to charge the second wearable device 2000b (e.g., the body 2100). For example, referring to FIG. 36, the wearable charging structure 3523 may be implemented to have various shapes of a housing such as a cylinder, and may be simultaneously charged by different charging structures in a state in which the wearable devices (e.g., the first wearable device 2000a and the second wearable device 2000b) are mounted on the housing.

In an embodiment, referring to 3601 of FIG. 36, the main body charging structure 3523a may be formed in a first region of the trough structure, and the strap charging structure 3523b may be formed in a second region spaced apart from the first region. Each of the two charging structures (e.g., the main body charging structure 3523a and the strap charging structure 3523b) may be implemented to include at least one magnet, and each of the two charging structures (e.g., the main body charging structure 3523a and the strap charging structure 3523b) may be implemented to be coupled to a charging terminal of the body 2100 and the first strap 2120a based on the at least one magnet to perform charging.

In another embodiment, referring to 3603 of FIG. 36, the strap charging structure 3523b may be implemented in a form connected by a wiring L from the housing of the wearable charging structure 3523. Accordingly, the wearable charging structure 3523 may be coupled to the charging terminal of the first strap 2120a while the body 2100 is disposed on the main body charging structure 3523a, so that charging may be performed at the same time.

According to various embodiments, the other device 3530 may include a display 3531, a communication circuit 3533, and a processor 3535, and is not limited to the described example and/or the illustrated example, and may be implemented to include more electronic components and/or fewer electronic components. The charging device 3500 (e.g., the processor 3535) may set a communication connection with another external device (e.g., the server 10, the wearable device 20, and the user terminal 30) using the communication circuit 3533, and provide results of medicine performance information received from the external device through the display 3531.

### 3.3.2. Operation Method of a Charging Device

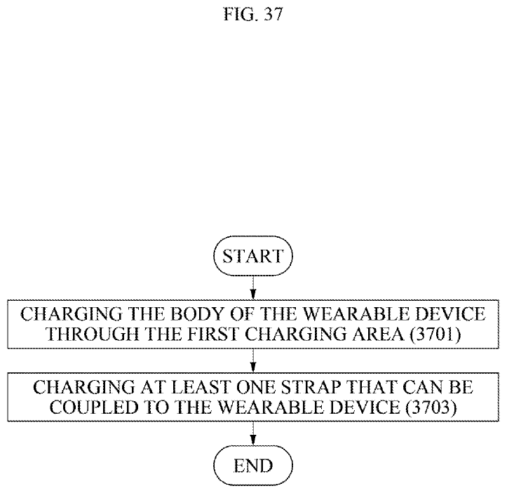

FIG. 37 is a flowchart illustrating operations of the charging device 3500 according to various embodiments. Operations may be performed regardless of the order of operations shown and/or described, and more operations may be performed and/or fewer operations may be performed.

According to various embodiments, the charging device 3500 (e.g., the processor 3535) may charge the body 2100 of the wearable device 20 through the first charging area (e.g., the main body charging structure 3523a) in operation 3701, and charge at least one strap (e.g., the first strap 2120a) that can be coupled to the wearable device 20 through the second charging area (e.g., the strap charging structure 3523b) in operation 3703.

### 4. Hybrid Medication Determination Operation

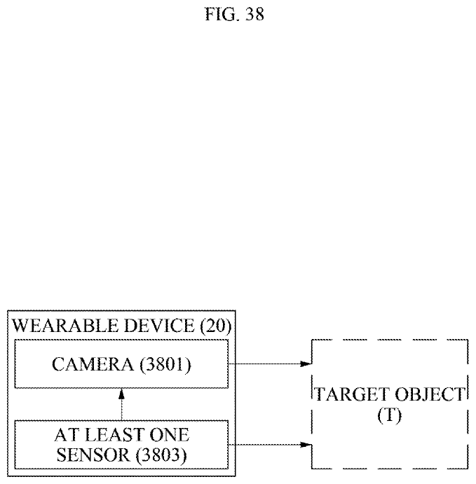

FIG. 38 is a block diagram illustrating an operation of performing a medication determination by further using at least one sensor 3803 of the wearable device 20 according to various embodiments.

According to various embodiments, referring to FIG. 38, the wearable device 20 may activate a camera 3801 for capturing a medication performance image based on a value identified by using the at least one sensor 3803 without the need for an activation signal received from the wireless communication device 40 (vision activation), and/or use the acquired information in at least a part of an operation of acquiring information about the medication performance by using the at least one sensor 3803 and acquiring information about the result of the medication determination (non vision algorithm).

According to various embodiments, the at least one sensor 3803 may include a motion sensor for sensing movement of an object, a microphone for sensing sounds generated from the outside, a vibration sensor for measuring vibrations of the object, a magnetic field sensor for measuring a magnetic field around the object, a proximity sensor for measuring a proximity to the object, a biometric information acquisition sensor such as an ultrasonic sensor, a biometric information acquisition sensor for measuring biometric information such as a electromyography sensor, and a vision limiting sensor such as a lidar, an IR sensor, and other optical sensor. When the medication performance image is captured by the camera 3801, a privacy problem may occur as sensitive objects that may leak personal information may occur due to the capturing may occur, and unlike the camera 2801, the at least one sensor 3803 has an advantage of preventing leakage of personal information.

According to various embodiments, the at least one sensor 3803 may be a sensor included in a second wearable device 200b (e.g., the body 2110) coupled with the add-on wearable device (e.g., the first wearable device 200a) described in the above “3. Class” but is not limited to the described example and may be a sensor included in a single wearable device (e.g., a smart watch).

### 4.1. Vision Activation Algorithm

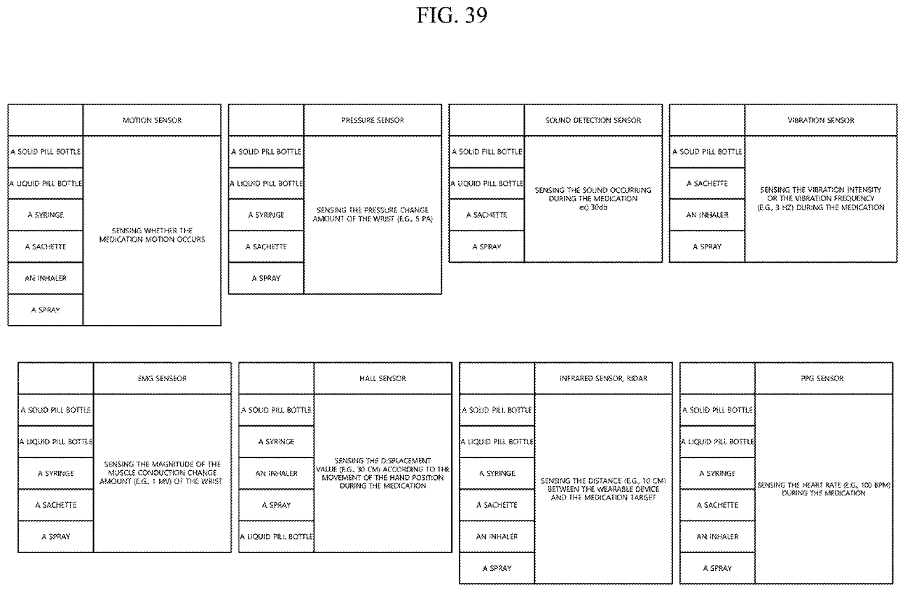

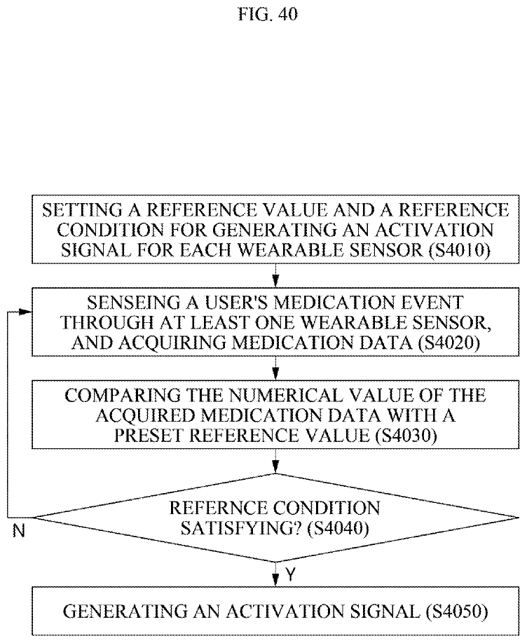

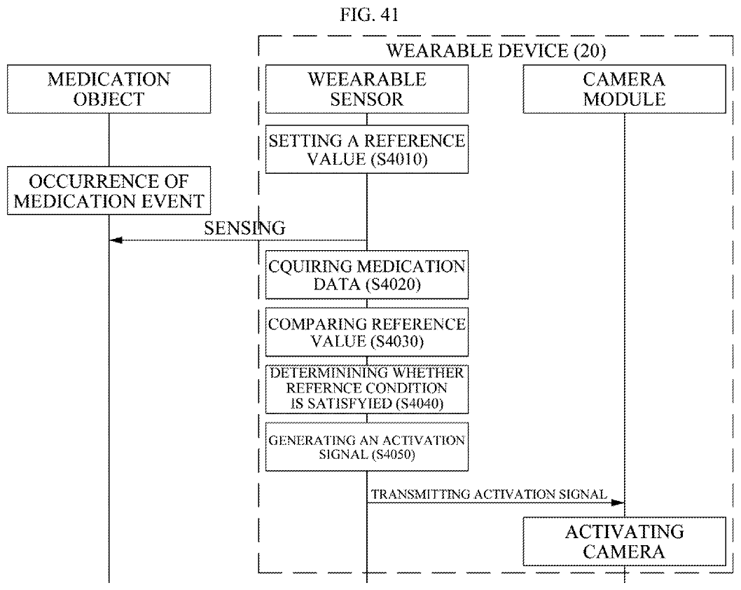

FIG. 39 is a diagram illustrating an activation signal reference condition for each wearable sensor in some embodiments of the present disclosure, FIG. 40 is a flowchart illustrating a process of generating an activation signal using a wearable sensor in some embodiments of the present disclosure, and FIG. 41 is a flowchart illustrating a process of generating an activation signal using a wearable sensor in some embodiments of the present disclosure. Hereinafter, a method of generating an activation signal will be described with reference to FIG. 39 to FIG. 41.

The wearable sensor is a sensor provided at one side of the wearable device 20 to generate an activation signal for a camera module through sensing of a medication target (e.g., a target device T), and may be various types of sensors that sense a medication target (e.g., a target device T) using a non-vision method.

Referring to FIG. 39, the wearable sensor may be a motion sensor that generates an activation signal based on a user's operation, a pressure sensor that generates an activation signal based on pressure data, a sound detection sensor that generates an activation signal based on sound data, a vibration sensor that generates an activation signal based on vibration data, an electromyography (EMG) sensor that generates an activation signal based on electromyography data, a Hall sensor that generates an activation signal based on three-dimensional magnetic data, an infrared ray (IR) sensor that generates an activation signal based on an electromagnetic wave in an infrared region, a Lidar that generates an activation signal based on a laser pulse, and a photoplethysmography (PPG) sensor that generates an activation signal based on heart rate data, and the medication target may be various types of medicinal containers such as a solid pill bottle, a liquid pill bottle, a syringe, a sachette, an inhaler, and a spray.

In some embodiments, the medication target (e.g., the target object T) may correspond to each according to a function of each wearable sensor. For example, the medication target (e.g., the target object T) sensed by the motion sensor may be at least one of a solid pill, a liquid pill, a syringe, an encapsulating agent, an inhaler, and a spray capable of motion recognition, the medication target (e.g., the target object T) sensed by the pressure sensor may be at least one of a liquid pill, an encapsulating agent, a syringe, and a spray capable of detecting a pressure change, the medication target (e.g., the target object T) sensed by the sound detection sensor may be at least one of a solid pill, an encapsulating agent, and a spray that generates noise during the medication, the medication target (e.g., the target object T) sensed by the vibration sensor may be at least one of a solid pill, an encapsulating agent, an inhaler, and a spray capable of vibration during the medication, the medication target (e.g., the target object T) sensed by the EMG sensor may be at least one of a liquid pill, an encapsulating agent, a syringe, and a spray capable of acquiring electromyographic data, the medication target (e.g., the target object T) sensed by the hall sensor may be a solid pill, a liquid pill, a syringe, an encapsulating agent, an inhaler, and a spray capable of acquiring three-dimensional magnetic data, the medication target (e.g., the target object T) sensed by the infrared sensor and the laser may be at least one of a solid pill, a liquid pill, a syringe, an encapsulating agent, an inhaler, and a spray capable of measuring distance, and the medication target (e.g., the target object T) sensed by the PPG sensor may be at least one of a solid pill, a liquid pill, a syringe, an encapsulating agent, an inhaler, and a spray capable of measuring heart rate.

Referring to FIGS. 40 and 41, in step S4010, the wearable device 20 sets a reference value and a reference condition for generating an activation signal for each wearable sensor for various medication objects (e.g., target object T) through at least one wearable sensor.

Each of the plurality of medication objects (e.g., target object T) corresponds to each other according to a function of each wearable sensor as described above, and the reference value and the reference condition set according to the function of each wearable sensor may also vary for each wearable sensor. Hereinafter, the reference value and the reference condition set for each wearable sensor will be described with an example.

Referring again to FIG. 39, since the motion sensor senses motion recognition related to the medication of the user, the wearable sensor may set whether the medication motion occurs due to the hand movement of the user as the reference condition. In the case of the pressure sensor, since the pressure change is sensed, the pressure change amount (e.g., 5 Pa) of the wrist occurring during the medication may be set as the reference condition. In the case of the sound detection sensor, the magnitude (e.g., 30 dB) of the sound occurring during the medication may be set as the reference condition, in the case of the vibration sensor, the vibration intensity or the vibration frequency (e.g., 3 Hz) during the medication may be set as the reference condition, in the case of the EMG sensor, the magnitude of the muscle conduction change amount (e.g., 1 mV) of the wrist during the medication may be set as the reference condition, in the case of the hall sensor, the displacement value (e.g., 30 cm) according to the movement of the hand position during the medication may be set as the reference condition, in the case of the infrared sensor or the lidar, the distance (e.g., 10 cm) between the wearable device 20 and the medication target (e.g., target object T) may be set as the reference condition, and in the case of the PPG sensor, the heart rate (e.g., 100 bpm) during the medication may be set as the reference condition.

In step S4020, when a medication event occurs, the wearable device 20 senses a user's medication event through at least one wearable sensor, and acquires medication data generated as a sensing result. The medication data may include different information according to the wearable sensor, and may be different types of data. For example, the wearable device 20 may acquire motion data from the motion sensor, acquire pressure data from the pressure sensor, acquire sound data from the sound detection sensor, acquire vibration data from the vibration sensor, acquire muscle conduction data from the EMG sensor, acquire 3D position data of the medication target (e.g., the target object T) from the hall sensor, acquire distance data between the wearable device 20 and the medication target (e.g., the target object T) from the infrared sensor or the lidar, and acquire heart rate data from the PPG sensor.

The wearable device 20 compares the numerical value of the acquired medication data with a preset reference value (step S4030), determines whether the comparison result satisfies the reference condition (step S4040), and generates an activation signal if the reference condition is satisfied (step S4050). For example, when the medication data is pressure data having a pressure change amount of 5 Pa, the preset reference value is 4 Pa, and the reference condition is greater than the reference value, the wearable device 20 generates the activation signal since the reference condition is satisfied when the pressure change amount of the medication data is compared with the reference value. The generated activation signal is transmitted to the camera module and is controlled to activate the camera.

As described above, according to the embodiment of FIG.S. 39 to 41, the wearable device 20 compares the medication data acquired through the non-vision wearable sensor with the reference value through the processor in the wearable device 20, and generates the activation signal immediately according to the comparison result, so that the camera can be quickly activated without a time delay according to data transmission and reception with the server 10.

On the other hand, in various embodiments, the wearable device 20 may transmit the acquired medication data to the server 10. That is, the wearable device 20 may identify medication objects (e.g., target objects T) corresponding to the medication data using an artificial intelligence model, and compare a preset reference condition for each type of the identified medication objects (e.g., target objects T) with the medication data.

In this case, rather than comparing the numerical value of the medication data and the reference value based on the type of the wearable sensor, the accuracy of the medication event determination may be improved by comparing the medication data and the reference value for each medication object (e.g., target object T) by considering both the type of the wearable sensor and the type of the medication object (e.g., target object T), and the efficiency of the activation signal generation may also be improved.

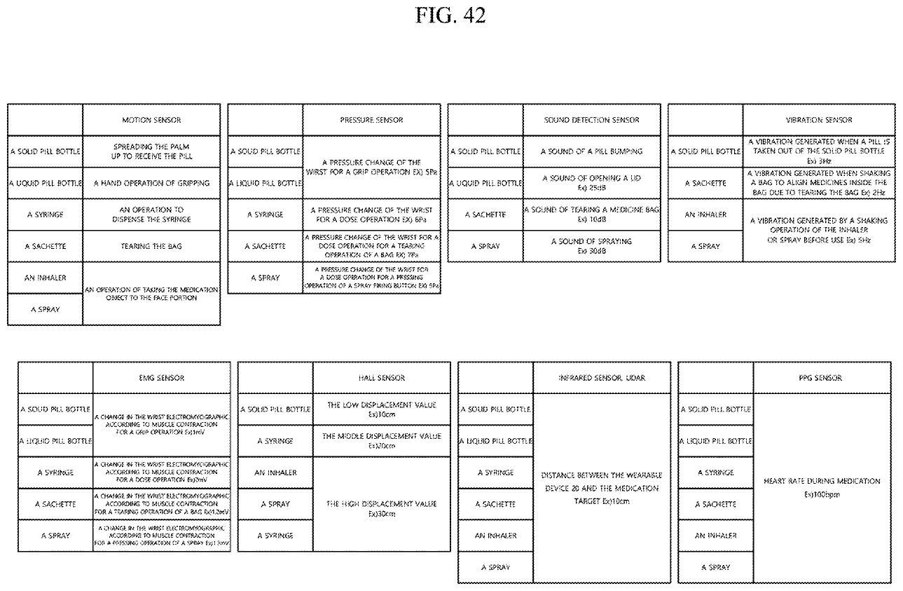

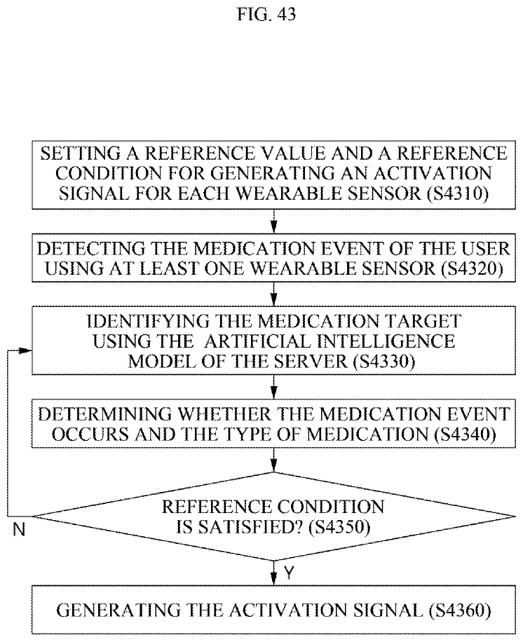

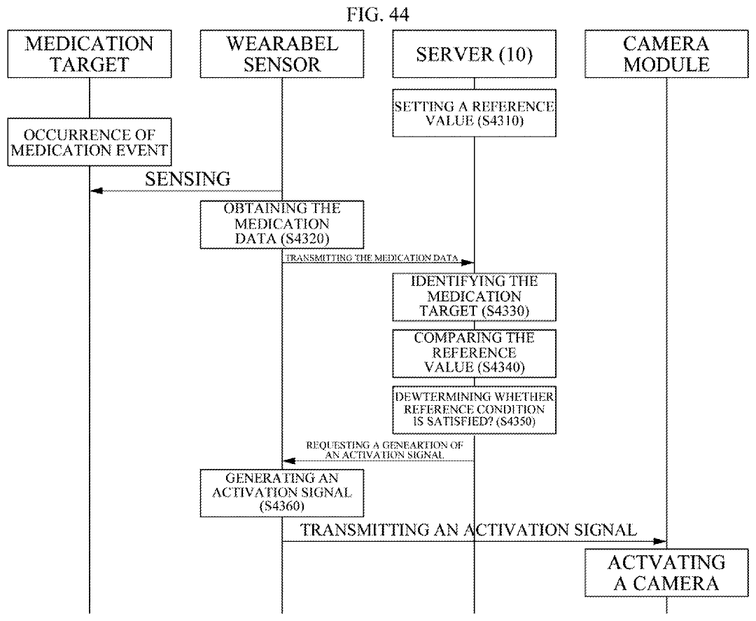

FIG. 42 is a diagram illustrating various types of wearable sensors and medication objects (e.g., target objects T) and activation signal reference conditions corresponding to each wearable sensor in some embodiments of the present disclosure, FIG. 43 is a flowchart illustrating a process of identifying medication objects (e.g., target objects T) and generating an activation signal through the wearable sensor and the server 10 in some embodiments of the present disclosure, and FIG. 44 is a flowchart illustrating a process of identifying medication objects (e.g., target objects T) and generating an activation signal through the wearable sensor and the server 10 in some embodiments of the present disclosure. A method of generating an activation signal will be described below with reference to FIGS. 42 to 44.

First, referring to FIGS. 43 and 44, in step S4310, the wearable device 20 sets a reference value and a reference condition for generating an activation signal for each wearable sensor for various medication objects (e.g., target object T) through at least one wearable sensor. In addition, the wearable device 20 may set a reference value and a reference condition for generating an activation signal for each medication object (e.g., target object T) as well as each wearable sensor based on the wearable sensor sensing different medication objects (e.g., target object T).

In various embodiments, each of the plurality of medication objects (e.g., target object T) corresponds to each other according to a function of each wearable sensor as described above, and the reference value and the reference condition set may also vary according to each wearable sensor as the functions of each wearable sensor are different. Hereinafter, the reference value and the reference condition set for each wearable sensor and medication object (e.g., target object T) will be described with an example.

Referring to FIG. 42, the medication object (e.g., target object T) sensed by the motion sensor may be a solid pill bottle, a liquid pill bottle, a syringe, a bag, an inhaler, and a spray that can recognize motion. In this case, the reference value (reference operation) for the motion sensor may be different according to the type of the medication object (e.g., target object T), such as an operation of spreading the palm up to receive the pill discharged from the solid pill bottle, a hand operation of gripping the liquid pill bottle, an operation of spreading the thumb and the index/drag finger to dispense the syringe to the body, an operation of tearing the bag, and an operation of taking the inhaler and the spray into the face portion (e.g., eye, nose, mouth) to use the medication object (e.g., target object T). The reference condition may be that each operation according to the medication object (e.g., target object T) lasts more than a specific time or that the reliability calculated in each successive operation will be more than a certain reliability.

The medication target (e.g., the target object T) sensed by the pressure sensor may be at least one of a medicine bottle, a bag, a syringe, and a spray capable of detecting a pressure change. In this case, the reference value for the pressure sensor may be a different wrist pressure change value due to muscle contraction in various operations such as a grip operation of a solid pill bottle or a liquid medicine bottle, a dose operation of a drug through a syringe, a tearing operation of a bag, and a pressing operation of a spray firing button, and the reference condition for the pressure sensor may be that the calculated wrist pressure change value is equal to or greater than the reference value for each operation.

The medication target (e.g., the target object T) sensed by the sound detection sensor may be at least one of a solid pill bottle, a bag, and a spray that generates noise during medication. In this case, the reference value for the sound detection sensor may be a magnitude, a frequency, and a waveform of various sounds commonly generated in each medication target (e.g., the target object T), such as a sound of a pill of a solid pill bottle, a sound of opening a lid of a medicine bottle, a sound of tearing a medicine bag, and a sound of spraying, and the reference condition for the sound detection sensor may be that the magnitude of the acquired noise is equal to or greater than the reference value for each medication target (e.g., the target object T).

The medication target (e.g., the target object T) sensed by the vibration sensor may be at least one of a solid pill bottle, a bag, an inhaler, and a spray that generates vibration during medication. In this case, the reference value for the vibration sensor may be a waveform, a strength, and a frequency of various vibrations generated in each medication target (e.g., the target object T), such as a vibration generated when a pill is taken out of the solid pill bottle, a vibration generated when shaking a bag to align medicines inside the bag due to tearing the bag, and a vibration generated by a shaking operation of the inhaler or spray before use, and the reference condition for the vibration sensor may be that the vibration waveform, the strength, and the frequency of the acquired vibration data are equal to or greater than the reference value for each medication target (e.g., the target object T).

The medication target (e.g., the target object (T)) sensed by the EMG sensor may be at least one of a medicine bottle, a bag, a syringe, and a spray capable of obtaining the electromyographic data. In this case, the reference value for the EMG sensor may be a change in the wrist electromyographic according to muscle contraction in various operations such as a grip operation of a solid pill bottle or a liquid medicine bottle, a dose operation of a drug through a syringe, a tearing operation of a bag from the bag, and a pressing operation of a spray firing button, and the reference condition for the EMG sensor may be that the change in the wrist electromyographic of the obtained electromyographic data is greater than or equal to the reference value for each of the medication target (e.g., the target object (T)).

The hall sensor measures a displacement value according to the positional movement of the wrist and senses the medication target (e.g., the target object (T)) according to the displacement value. The medication target (e.g., the target object (T)) sensed by the hall sensor may be a solid pill bottle, a liquid medicine bottle, a syringe, a bag, an inhaler, and a spray capable of obtaining the three-dimensional magnetic data. In this case, the reference value for the hall sensor may be a displacement value. In detail, the solid pill bottle has a small displacement value because the positional movement occurs around the hand, the syringe has a middle displacement value because the positional movement occurs as the wrist, and the like, and the inhaler, the spray, and the liquid stream are positional moved to the face portion, and thus may have a high displacement value. In addition, the reference condition for the hall sensor may be that the displacement valuel of the obtained three-dimensional magnetic data is greater than or equal to the reference value for each of the medication target (e.g., the target object (T)).

The medication target (e.g., the target object (T)) sensed by the infrared sensor or the lidar may be at least one of a solid pill bottle, a liquid medicine bottle, a syringe, a bag, an inhaler, and a spray capable of measuring the distance. In this case, the reference value for the infrared sensor and the lid may be a preset distance between the wearable device 20 and the medication target (e.g., the target object (T)), and the reference condition for the infrared sensor and the lid may be that the determined distance is greater than or equal to the reference value.

The medication target (e.g., target object T) sensed by the PPG sensor may be at least one of a solid pill bottle, a liquid bottle, a syringe, a bag, an inhaler, and a spray. In this case, the reference value for the PPG sensor may be a heart rate, and the reference range may be equal to or greater than the obtained heart rate.

In step S4320, the wearable device 20 detects the medication event of the user using at least one wearable sensor, and if the medication data is acquired, transmits the medication data to the server to identify the medication target (e.g., target object T) through the artificial intelligence (AI) model of the server in step S4330

Next, the server 10 compares the numerical value included in the medication data with the reference value corresponding to the identified medication target (e.g., target object T), determines whether the medication event occurs and the type of medication (step S4340), if the (step S4350), and generates the activation signal through the transmission of the activation signal generation request to the wearable device 20 (step S4360).

Unlike the above-described embodiment, in this embodiment, as the medication data analysis is performed through the artificial intelligence model of the server, the medication target (e.g., target object T) can be identified. Therefore, the reference value and the reference condition for the activation signal generation are not only set for each wearable sensor, but also set for each medication target (e.g., target object T), so it is possible to also confirm which kind of restrictions are medication as well as the medication timing of the user, and thus the efficiency of the activation signal generation can be improved.

### 4.2. Non-Vision Algorithm

### 4.2.1. Single Medication Determination Operation

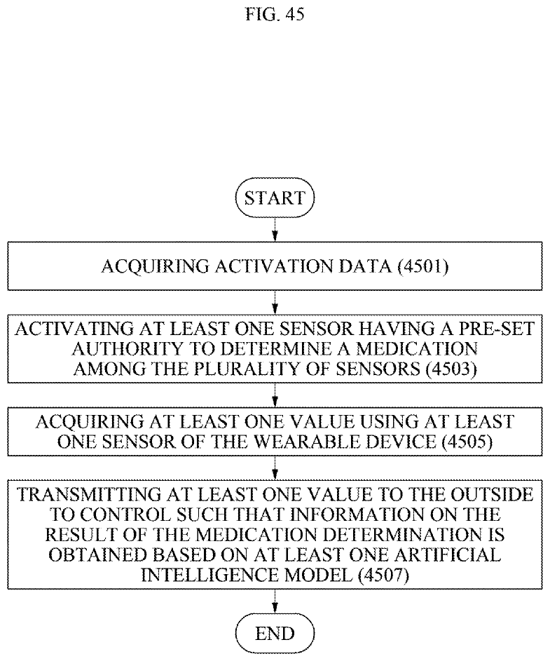

According to various embodiments, the health management system 1 may acquire information on taking performance using at least one sensor 3803 of the wearable device 20 and provide a taking determination result based on the acquired information on taking performance. The health management system 1 may acquire at least one artificial intelligence model by performing learning using information on the sensor value and the taking determination result corresponding to the sensor value as training data and perform a taking determination operation based on the at least one artificial intelligence model, which will be described below with reference to FIGS. 45 to 51.

FIG. 45 is a flowchart illustrating an example of an operation for providing a taking determination result based on at least one sensor 4620 of the wearable device 20 according to various embodiments. Operations may be performed regardless of the order of the operations shown and/or described, and more operations may be performed and/or fewer operations may be performed.

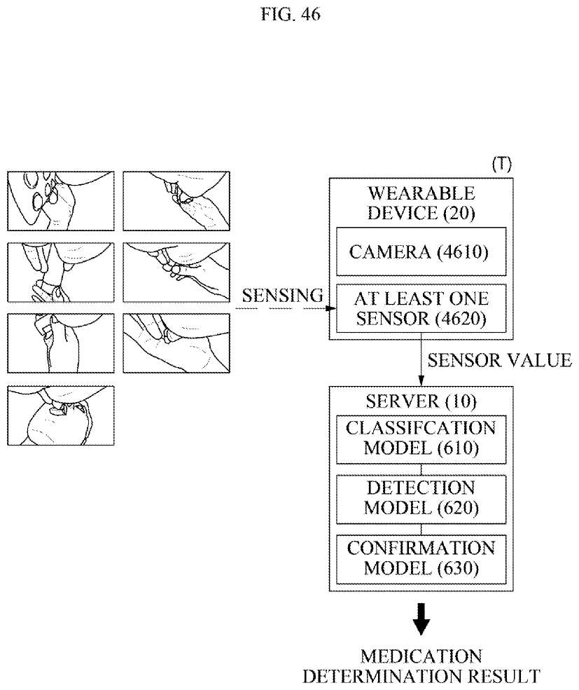

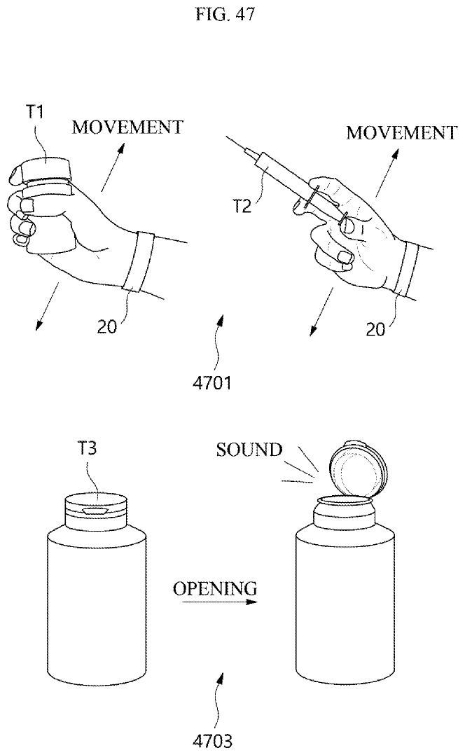

FIGS. 46 to 47 are diagrams illustrating an operation of acquiring a taking determination result based on the sensor value of the health management system 1 according to various embodiments.

According to various embodiments, the wearable device 20 may acquire activation data in operation 4501. In an embodiment, the wearable device 20 may receive activation data generated by the wireless communication device 40 generated based on the movement of the target object T when a user performs a taking. In another embodiment, the wearable device 20 may acquire activation data using at least one sensor 4620 (e.g., at least one sensor 3803), as described in “5.1 letter”.

According to various embodiments, the wearable device 20 may activate at least one sensor 4620 having a pre-set authority to determine a medication among the plurality of sensors in operation 4503, and may acquire at least one value using at least one sensor 4620 of the wearable device in operation 4505. For example, the wearable device 20 may activate at least one sensor 4620 (e.g., a physical information acquisition sensor, a biometric information acquisition sensor, and a vision information limiting sensor) that is set to acquire information on a medication performance among the plurality of sensors of the wearable device 20 based on receiving the activation data, and may acquire at least one sensor value using the activated at least one sensor 3803. The application (e.g., the first application 900) executed in the wearable device 20 may be set to have the authority to use the at least one sensor 4620 (e.g., the physical information acquisition sensor, the biometric information acquisition sensor, and the vision information limiting sensor) based on the user's consent of the wearable device 20.

According to various embodiments, as shown in FIG. 46, the wearable device 20 may be implemented to acquire non-vision information using at least one sensor 4620. For example, as shown in 4701 of FIG. 47, the wearable device 20 may acquire movements (or vibrations) generated by the drug performance of the user during the taking of the medication using physical information acquisition sensors (e.g., motion sensors, vibration sensors, and the like), and/or acquire biometric information (e.g., electromyogram information) of the user that is changed by the drug performance of the user using biometric information sensors. For example, as shown in 4703 of FIG. 47, the wearable device 20 may acquire sounds generated by the drug performance of the user during the taking of the medication through a microphone. Although not shown, for example, the wearable device 20 may acquire information about the taking of the medication using limited vision sensors (e.g., lidar, IR sensors, and the like).

According to various embodiments, as described above, the at least one sensor 4620 may be a sensor included in a second wearable device 200b (e.g., body 2110) coupled with the add-on wearable device (e.g., first wearable device 200a) described in “3. Class” described above, but is not limited to the examples described and may be a sensor included in a single wearable device 20 (e.g., a smart watch).

According to various embodiments, in operation 4507, the wearable device 20 may transmit at least one value to the outside (e.g., the server 10 or the user terminal 30) to control such that information on the result of the medication determination is obtained based on at least one artificial intelligence model (e.g., at least one of the classification model 610, the detection model 620, or the confirmation model 630). For example, the server 10 may receive at least one value obtained by the wearable device 20 and acquire the medication determination result information in response to inputting the received at least one value to at least one pre-trained artificial intelligence model (e.g., at least one of the classification model 610, the detection model 620, or the confirmation model 630) to acquire the result of the medication determination based on non-vision information. The medication determination result information may be provided through the user terminal 30 and/or the wearable device 20 as described above. Hereinafter, examples of learning and/or using at least one artificial intelligence model for providing the result of the medication determination based on non-vision information of the server 10 according to various embodiments will be described.

### 4.2.2. Medication Determination Based on a Single Sensor

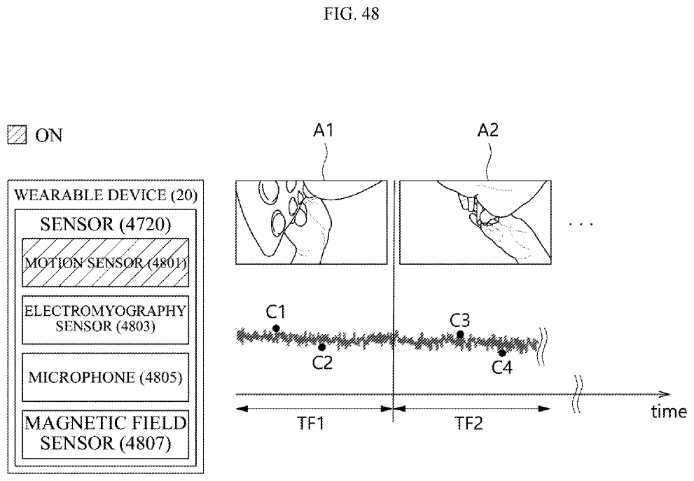

FIGS. 48 to 51 are diagrams illustrating examples of learning an artificial intelligence model for one sensor-based medication determination and determining medication based on the learned artificial intelligence model according to various embodiments.

According to various embodiments, the server 10 may learn at least one artificial intelligence model (e.g., the classification model 620, the confirmation model 630, or a single artificial intelligence model (not shown)) based on a sensor value received from sensors included in the wearable device 20 (e.g., motion sensor (e.g., acceleration sensor), electromyography sensor, microphone, magnetic field sensor, or other sensor (not shown) (e.g., vibration sensor, ultrasonic sensor). Based on the at least one artificial intelligence model, a medication determination operation may be performed.

### 4.2.2.1. Multiple Artificial Intelligence Model

Hereinafter, the description will be given with reference to FIGS. 48 to 50.

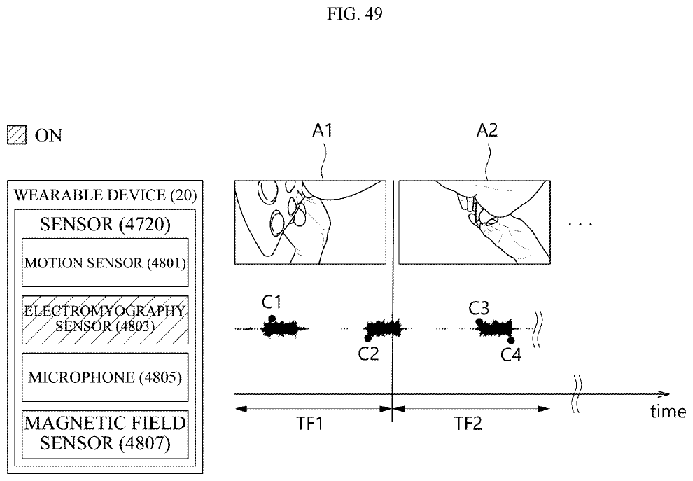

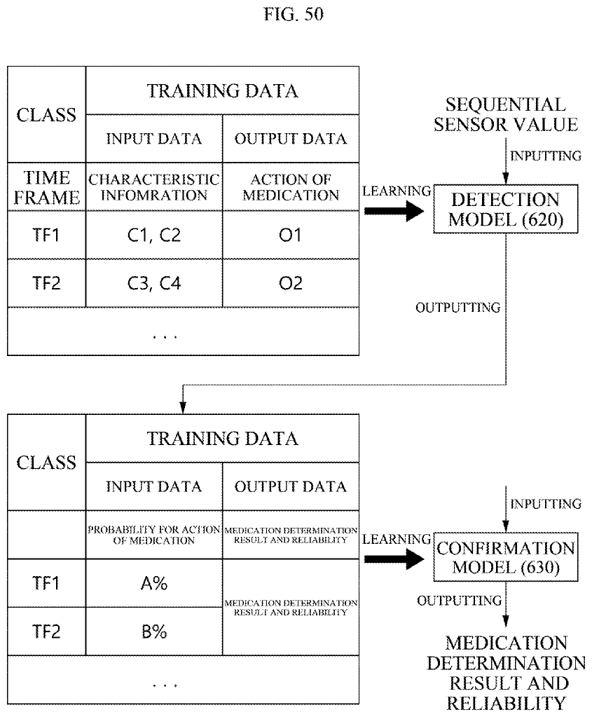

According to various embodiments, as the user performs the medication while wearing the wearable device 20, the server 10 may generate at least one artificial intelligence model based on the characteristic values of the sensor values sequentially (or time seriesly) from the sensors (e.g., motion sensors (e.g., acceleration sensors), electromyographic sensors, microphones, magnetic field sensors, or other sensors (e.g., vibration sensors, ultrasonic sensors), which are not shown) of the wearable device 20. For example, referring to FIGS. 48 to 49, as the user performs the medication while wearing the wearable device 20, the sensor values (e.g., angular velocity values, electromyographic values, sound values, and magnetic field values for each axis) may be sequentially (or time seriesly) acquired from the sensors (e.g., motion sensors (e.g., acceleration sensors), electromyographic sensors, microphones, magnetic field sensors, or other sensors (e.g., vibration sensors, ultrasonic sensors), which are not shown) of the wearable device 20. The characteristic information (C1, C2, C3, and C4) may be extracted (or labeled) for each time frame (TF1, TF2) of the acquired sequential sensor values. The characteristic information (C1, C2, C3, and C4) may indicate information that is characteristic of the sensor value, such as a characteristic value of the sensor value, an inflection point of the sensor value, and a slope of the sensor value. In an embodiment, as shown in FIG. 50, the server 10 may generate the classification model 610 by learning the extracted (or labeled) characteristic values (C1, C2, C3, and C4) of the sensor values as input data and a category index indieating a category of the medication behavior actually performed by the user as output data. The classification model 610 may be implemented to output the category index based on the input of the sequential sensor data acquired by the wearable device 20. In another embodiment, the server 10 may generate a detection model 620 by learning feature values C1, C2, C3, and C4 for each of the time frames TF1, TF2 as input data and the actions of the medication O1 and O2 performed by users corresponding to each of the time frames TF1, TF2 as output data. For example, when the category of medication performance is an act of taking and eating pills contained in a drug pain, the first time frame TF1 may correspond to the first action of the medication O1 (e.g., taking the pill), and the second time frame TF2 may correspond to the second action of the medication O2 (e.g., taking the pill day). That is, feature information C1, C2, C3, and C4 for each of the time frames TF1, TF2 may indicate the actions O1 and O2. The detection model 620 may be implemented to output probability information of the action of the medication for each of the time frames based on the input of the sequential motion data acquired by the wearable device 20, as shown in FIG. 50.

According to various embodiments, the result information output from the detection model may be different based on the category index. As described above, different detection models are implemented and selected for each category index, and/or the detection model 610 is implemented so that the category index can be input together with motion data, the probability information of the drug compliance action obtained from the detection model 620 may be different according to the category index.

According to various embodiments, as shown in FIG. 50, the server 10 may learn the confirmation model 630 implemented to output the result of determining the medication that indicates whether to perform the medication of a specific category in response to receiving the probability information of the drug compliance action.

As a result, according to various embodiments, the server 10 may obtain the result of determining the medication information based on inputting the sequential sensor value received from the wearable device 20 to at least a part of the plurality of artificial intelligence models (e.g., the classification model 610, the detection model 620, and the confirmation model 630).

### 4.2.2.2. Single Artificial Intelligence Model

Meanwhile, the server 10 is not limited to the above example, but may implement a single artificial intelligence model (not shown) that outputs the result of the medication determination in response to the input of motion data by setting characteristic information (C1, C2, C3, and C4) for each time frame (TF1, TF2) of the sensor value as input data and setting the result of the medication determination as output data to perform learning. In this case, the classification model may be separately implemented, and the artificial intelligence model (not shown) may be implemented to provide different results according to identification information (e.g., category index) for the category of the medication determination, but is not limited to the examples described.

### 4.2.3. Medication Determination Based on Multiple Sensors

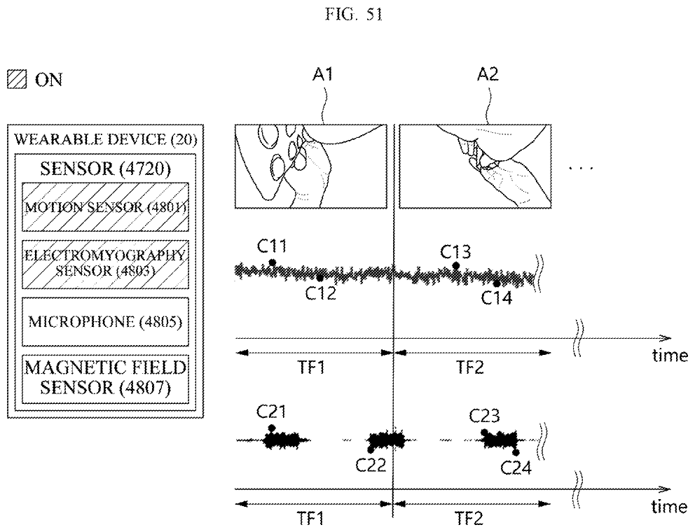

FIG. 51 is a diagram illustrating an example of learning an artificial intelligence model for determining a medication based on a plurality of sensors and determining a medication based on the learned artificial intelligence model, according to various embodiments.

According to various embodiments, as shown in FIG. 51, the server 10 may learn at least one artificial intelligence model for providing a medication determination result based on sensor values obtained from a plurality of sensors (e.g., the motion sensor 4801 and the electromyography sensor 4803) of the wearable device 20 and perform an operation of obtaining medication determination result information based on the pre-learned at least one artificial intelligence model. In this case, since the learning of the at least one artificial intelligence model may be performed like the above-described “5.2.2 single sensor-based medication determination operation”, redundant descriptions will be omitted.

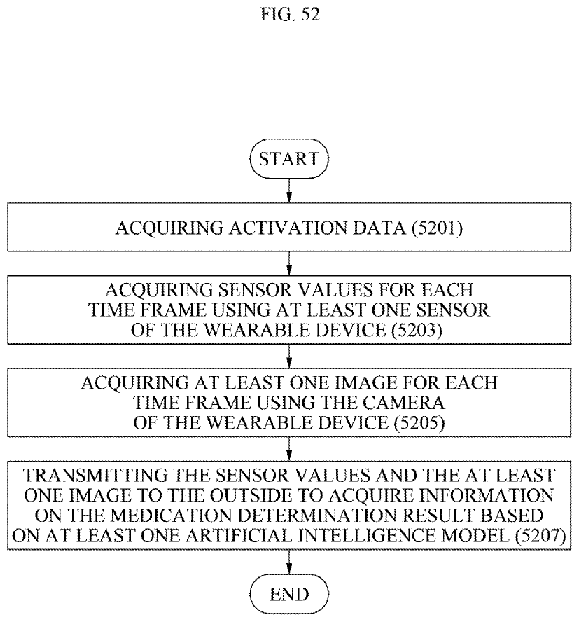

According to various embodiments, referring to FIG. 52, the server 10 may use different kinds of sensor values (e.g., motion data and electromyography data) obtained simultaneously from each of the plurality of sensors (e.g., the motion sensor 4801 and the electromyography sensor 4803) when performing a medication by the user as input data in comparison with the single sensor-based medication determination operation. Each of the sensor values may be extracted by time frames TF1, TF2, and feature information C11, C12, C21, C22, C13, C14, C23, C24 may be extracted. In addition, the learning of the classification model based on the extracting (labeling) of the feature values and the corresponding drug performance operation and/or the detection model may be performed like the “5.2.2”. Since the learning of the single sensor-based medication determination operation may be performed like the single sensor-based medication determination operation, redundant descriptions will be omitted.

### 4.2.4. Composite Medication Determination

According to various embodiments, the server 10 may acquire medication determination result information based on the medication performance image captured by the camera 4610 of the wearable device 20 and the sensor value acquired from the aforementioned sensors 4620.

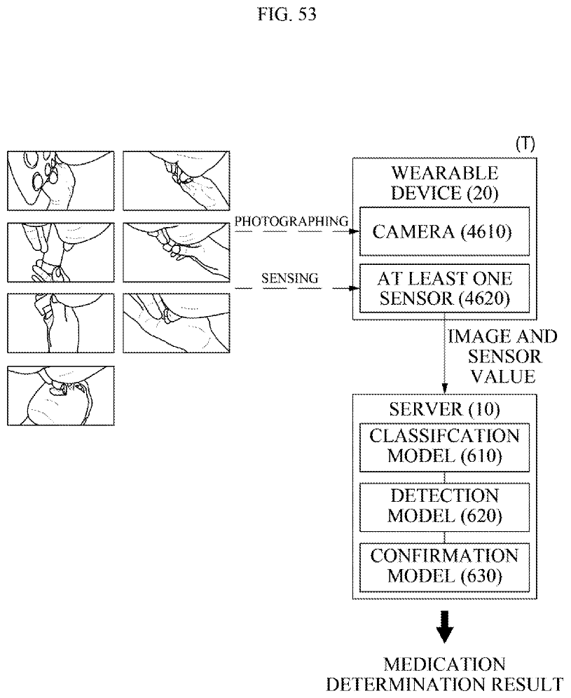

FIG. 52 is a flowchart illustrating an example of a complex medication determination operation of the wearable device 20 according to various embodiments. Operations may be performed regardless of the order of the operations shown and/or described, and more operations may be performed and/or fewer operations may be performed. Hereinafter, FIG. 52 will be further described with reference to FIG. 53.

FIG. 53 is a diagram illustrating an example of a complex medication determination operation of the server 10 according to various embodiments.

According to various embodiments, in operation 5201, the wearable device 20 may acquire activation data.

According to various embodiments, the wearable device 20 may acquire sensor values for each time frame using at least one sensor 4620 of the wearable device 20 in operation 5203, and acquire at least one image for each time frame using the camera 4610 of the wearable device 20 in operation 5205. For example, as shown in FIG. 53, the wearable device 20 may acquire medication performance images and sensor values simultaneously based on activating the camera 4610 and the at least one sensor 4620 based on receiving the activation data.

According to various embodiments, in operation 5207, the wearable device 20 may transmit the sensor values and the at least one image to the outside (e.g., the server 10, and/or the user terminal 30) to acquire information on the medication determination result based on at least one artificial intelligence model.

### 4.2.4.1. Determination at Different Times

According to various embodiments, the server 10 may be implemented to use the sensor value obtained by the wearable device 20 and the different artificial intelligence model for each of the at least one image. For example, as shown in Table 1, the server 10 may finally obtain medication determination result information based on the result information obtained in response to the input of the sensor value or the at least one image to the artificial intelligence model.

TABLE 1

| Embodiments | Classification model | Detection model |
|---|---|---|
| First embodiment | Specific sensor value | Medication image |
| Second embodiment | Medication image | Specific sensor value |

As an example, in the case of the first embodiment of Table 1, the server 10 may select a detection model based on a category index output based on the input of the specific sensor value to the classification model and input information on a target object (t) output based on the input of the medication image to the detection model to a confirmation model, thereby obtaining resultant medication determination result information. As another example, in the case of the second embodiment of Table 1, the server 10 may select a detection model based on a category index output based on the input of the medication image to the classification model and input probability information on a drug performance operation output based on the input of the sensor value to the detection model to a confirmation model, thereby obtaining resultant medication determination result information.

### 4.2.4.2. Determination at Same Time

According to various embodiments, the server 10 may be implemented to use the sensor value and the at least one image acquired by the wearable device 20 together in a specific artificial intelligence model. To this end, each of the classification model and the detection model may be used as input data, a sensor value corresponding to the medication performance image together with the medication performance image. At this time, learning of the classification model and the detection model may be performed based on labeling of the medication performance image for each time frame and feature values of the sensor value for each time frame, and learning operations for each model may be performed as described above, and thus detailed descriptions are omitted.

### 4.2.5. Medication Determination Scenario

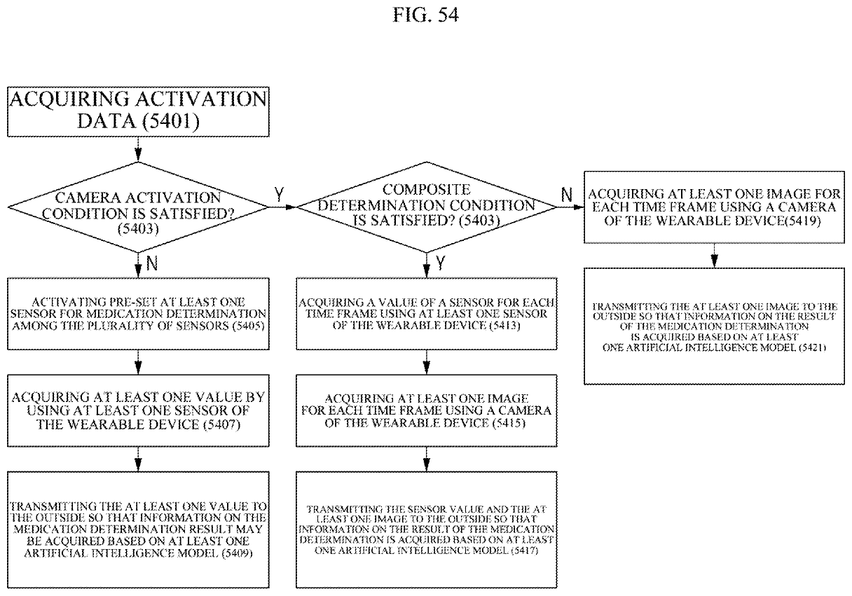

FIG. 54 is a flowchart illustrating an example of a medication determination scenario of the wearable device 20 according to various embodiments. Operations may be performed regardless of the order of the operations shown and/or described, and more operations may be performed and/or fewer operations may be performed.

According to various embodiments, the wearable device 20 may acquire activation data in operation 5401.

According to various embodiments, the wearable device 20 may determine whether the camera activation condition is satisfied in operation 5403, and when the camera activation condition is not satisfied (5403-N), activate at least one sensor pre-set (authority) for medication determination among the plurality of sensors in operation 5405, acquire at least one value by using at least one sensor of the wearable device in operation 5407, and transmit the at least one value to the outside so that information on the medication determination result may be acquired based on at least one artificial intelligence model in operation 5409. That is, in a situation where the camera 4610 is difficult to use, the wearable device 20 may be set to acquire the value of the sensor 4620, and may perform an operation so that the medication determination result information may be acquired based on the value of the sensor 4620.

According to various embodiments, when the camera activation condition is satisfied (5403-Y), the wearable device 20 may determine whether the composite determination condition is satisfied in operation 5411, when the composite determination condition is satisfied (5411-Y), acquire a value of a sensor for each time frame using at least one sensor of the wearable device in operation 5413, acquire at least one image for each time frame using a camera of the wearable device in operation 5415, and transmit the sensor value and the at least one image to the outside so that information on the result of the medication determination is acquired based on at least one artificial intelligence model in operation 5417. That is, in a situation where the wearable device 20 can use the camera 4610, but the accuracy of the medication determination is less than a threshold, so that the cooperation of the sensor 4620 is necessary, the wearable device 20 may be set to acquire the value of the sensor 4620 together with the medication performance image using the camera 4610, so that the medication determination result information is acquired based on the medication performance image and the value of the sensor 4620.

According to various embodiments, when the composite determination condition is not satisfied (5411-N), the wearable device 20 may acquire at least one image for each time frame using a camera of the wearable device in operation 5419, and transmit the at least one image to the outside so that information on the result of the medication determination is acquired based on at least one artificial intelligence model in operation 5421. That is, in a situation where the wearable device 20 can use the camera 4610, and the accuracy of the medication determination is greater than or equal to the threshold, the wearable device 20 may be set to acquire the medication performance image using the camera 4610, so that the medication determination result information is acquired based on the medication performance image.

### 5. User Scenario

According to various embodiments, the health management system 1 basically provides a service that enhances the accuracy of taking a medicine performance of a user using the health management system 1, and may provide various types of services for ultimately enhancing the overall quality of life of the user by linking taking a medicine determination result information and other biometric information.

### 5.1. Fine Medication Management

### 5.1.1. Medication Record and Alarm

According to various embodiments, the health management system 1 automatically records the results of the medication execution performed by the user, and provides a continuous alarm even when the user does not perform the medication, so that the user can perform the medication without missing.

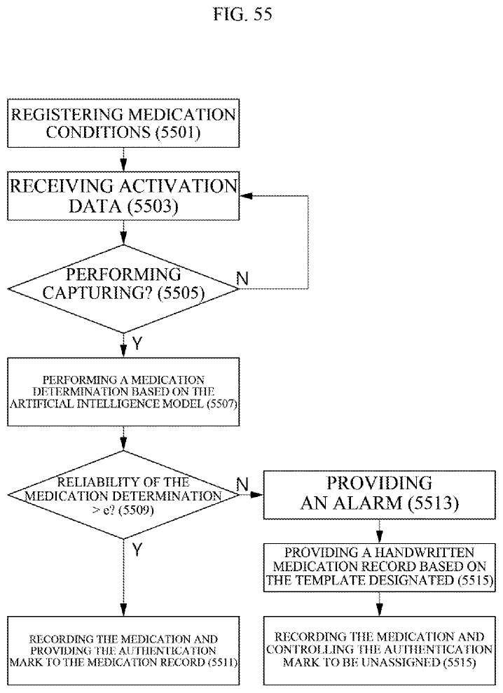

FIG. 55 is a flowchart illustrating an example of the medication record and alarm operations of the health management system 1 according to various embodiments. Operations may be performed regardless of the order of the operations shown and/or described, and more operations may be performed and/or fewer operations may be performed.

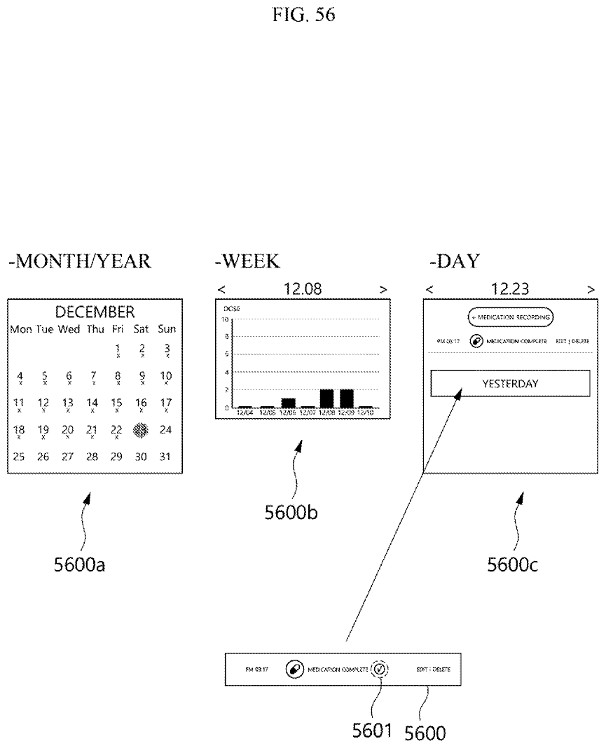

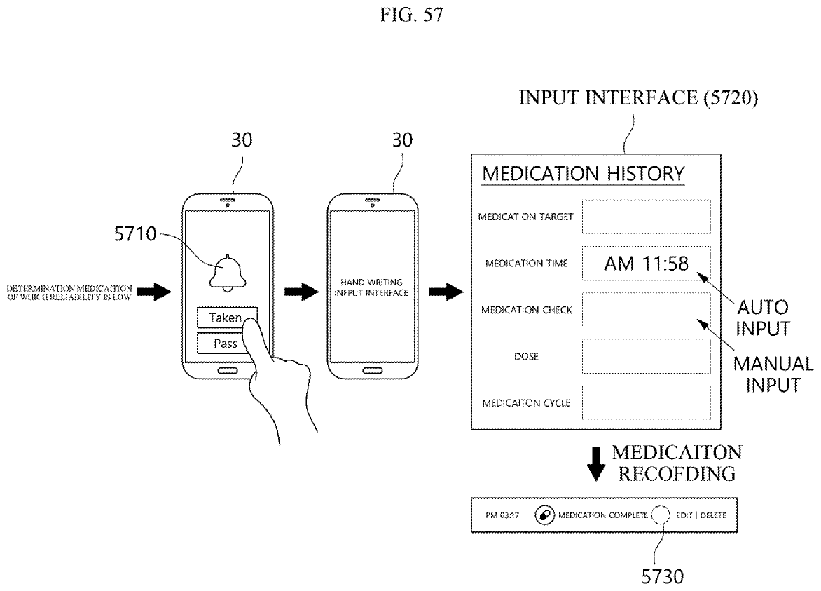

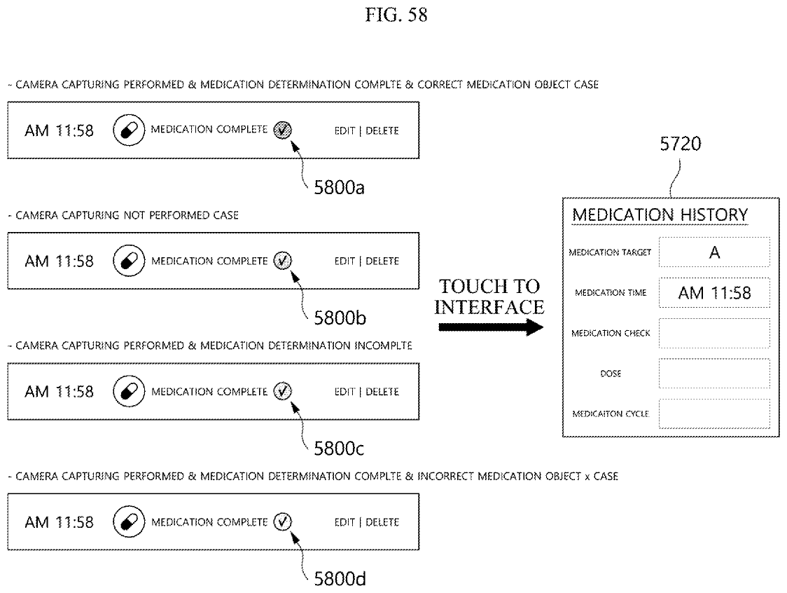

FIG. 56 is a diagram illustrating an example of an execution screen including medication determination result information according to various embodiments. FIG. 57 is a diagram illustrating an example of handwriting input when not performing medication according to various embodiments. FIG. 58 is a diagram illustrating an example of un-authentication of medication when handwriting input when not performing medication according to various embodiments.

According to various embodiments, the health management system 1 (e.g., the wearable device 20) may register medication conditions in operation 5501, receive activation data in operation 5503, and determine whether to perform photographing in operation 5503. For example, the wearable device 20 may determine whether the RSSI of the wireless signal received from the wireless communication device 40 is equal to or greater than a pre-set value as at least a part of the operation of determining whether to perform photographing, and/or determine whether the shielding structure in front of the camera of the wearable device 20 is open. The wearable device 20 may determine to perform photographing when the RSSI of the wireless signal is equal to or greater than the pre-set value and/or the shielding structure is open.

According to various embodiments, the health management system 1 (e.g., the server 10) may perform a medication determination based on the artificial intelligence model in operation 5507 when it is determined to perform photographing (5505-Y). The server 10 may obtain the reliability of the medication determination result information and the medication determination result information based on inputting the medication performance image photographed by the wearable device 20 to at least one artificial intelligence model (e.g., the classification model 610, the detection model 620, and the confirmation model 630).

According to various embodiments, the health management system 1 (e.g., the server 10) may identify whether the reliability of the medication determination exceeds the threshold (c) in operation 5509, and may control the medication record and the authentication mark to be given in operation 5511 when the reliability of the medication determination exceeds the threshold (5509-Y). For example, the server 10 may determine whether the reliability of the medication determination result information is equal to or greater than the threshold, determine that the medication determination result information is authenticated when the reliability is equal to or greater than the threshold, and determine that the medication determination result information is unauthenticated when the reliability is less than (or equal to or less than) the threshold.

According to various embodiments, the local device (e.g., the wearable device 20 and/or the user terminal 30) may provide information and/or authentication marks about a medication on the execution screen of the application (e.g., the first application 900 and the second application 1000) based on whether to authenticate the medication determination result information. The information about the medication may include at least one of whether or not a pre-set medication goal (e.g., the number of medications, the medication cycle, and the medication time) is achieved, the number of medications, the medication time, or the medication object (e.g., the type of medicine) (or the type of medication (e.g., the inhaler, the pill, etc.). For example, the execution screen 1000 of the second application of the user terminal may include information about a medication per year/month (e.g., whether or not a medication goal per day) as illustrated in 5600a of FIG. 56 or may include information about a medication per week (e.g., the number of medication per day per week) as illustrated in 5600b of FIG. 56 and information about a medication per day (e.g., the medication time, the type of medication) as illustrated in 5600c of FIG. 56 based on the medication determination result information of the server 10. In this case, as described above, the authentication mark 5601 may be provided on the execution screen 1000 of the second application in a form associated with the information 5600 about the medication corresponding to the medication determination result information authenticated by the server 10. Alternatively, the authentication mark 5601 may not be assigned to the unauthenticated medication determination result information as illustrated in 5730 of FIG. 57. Accordingly, the user may recognize the reliability of the information 5600 about the automatically recorded medication and double check the contents of the information 5600 about the medication to improve the accuracy of the medication record.

According to various embodiments, the authentication mark for the medication information 5600 may be implemented to be variously given for each case. In an embodiment, as shown in FIG. 58, if the medication performance image is captured by the camera of the wearable device, and the reliability of the medication determination result information is equal to or higher than a pre-set value (completion of medication determination), and the medication performance for the pre-set medication target, the first authentication mark 5800a may be given. In addition, in the case of incomplete medication performance (e.g., uncaptured camera, uncompleted medication determination, and not medication target), different types of unauthenticated marks 5800b, 5800c, and 5800d may be given. Accordingly, depending on the type of the unauthenticated mark for the recorded medication information, the user may identify the insufficient medication information (e.g., medication time, medication target, and so on) for the corresponding medication record, and may input the insufficient medication information with hand.

According to various embodiments, the health management system 1 (e.g., the server 10) may provide an alarm in operation 5513 when the reliability of the medication determination is less than or equal to the threshold (e) (5509-N), provide a handwritten medication record based on the template designated in operation 5515, and record the medication and control the authentication mark to be unassigned in operation 5517. For example, referring to FIG. 56, if information about the medication determination result of the reliability of the user's local device (e.g., the user terminal 30 and/or the wearable device 20) is identified less than or equal to a pre-set value, the user's local device (e.g., the user terminal 30 and/or the wearable device 20) may provide an alarm 5710 about the reliability of the medication determination result. The local device (e.g., the user terminal 30 and/or the wearable device 20) may provide an input interface 5720 for inputting information about the medication by hand based on receiving a user's input (e.g., an alarm screen touch, handwritten input menu touch) associated with the alarm 5710. The input interface 5720 may include an input graphic object (e.g., a text field, a file attachment object) for inputting information about the medication according to the type of the medication. In this case, the input interface 5720 may be implemented to include information about a specific type of medication already automatically input (e.g., medication time) and to receive information about insufficient medication (e.g., whether the medication is actually given, a medication target, a medication quantity, and a medication cycle) manually input based on the input graphic object. For example, the medication time may be automatically input at the time when activation data is received in the wearable device 20, the time when the medication execution image is captured by the wearable device 20 is completed, or the time when the medication determination result information is acquired by the server 10.

According to various embodiments, the health management system 1 (e.g., the server 10) may provide an execution screen for hand-held medication recording through a local device (e.g., the wearable device 20 and/or the user terminal 30) separately from the photographing of the camera of the wearable device 20, and if information on medication execution is input through the execution screen, the health management system 1 may record information on medication execution, but control the medication execution to be unauthenticated (e.g., an authentication mark is not given).

### 5.1.2. Medication Remind

According to various embodiments, the health management system 1 may automatically record the results of the performance of the medication performed by the user, and provide a continuous alarm even when the user does not perform the medication, so that the user may perform the medication without missing.

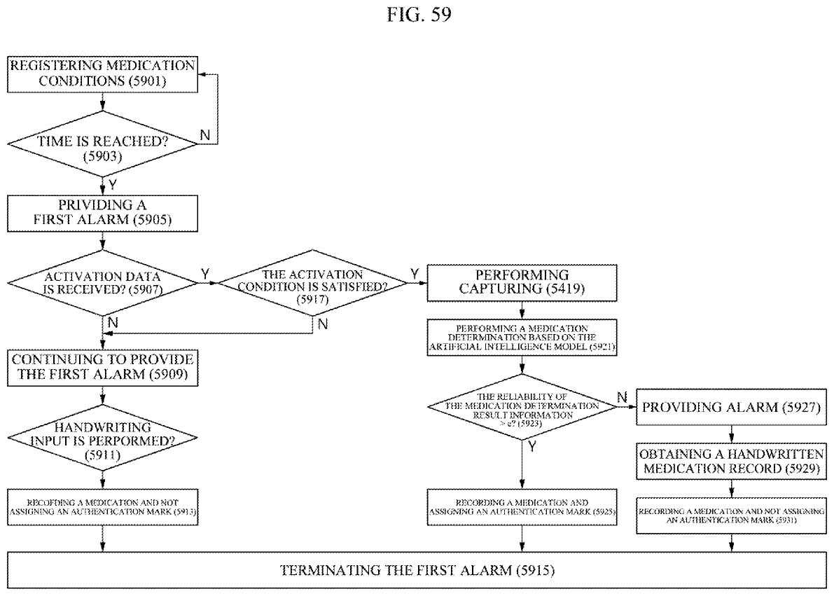

FIG. 59 is a flowchart illustrating an example of an operation of the medication reminder of the health management system 1 according to various embodiments. Operations may be performed regardless of the order of the operations shown and/or described, and more operations may be performed and/or fewer operations may be performed.

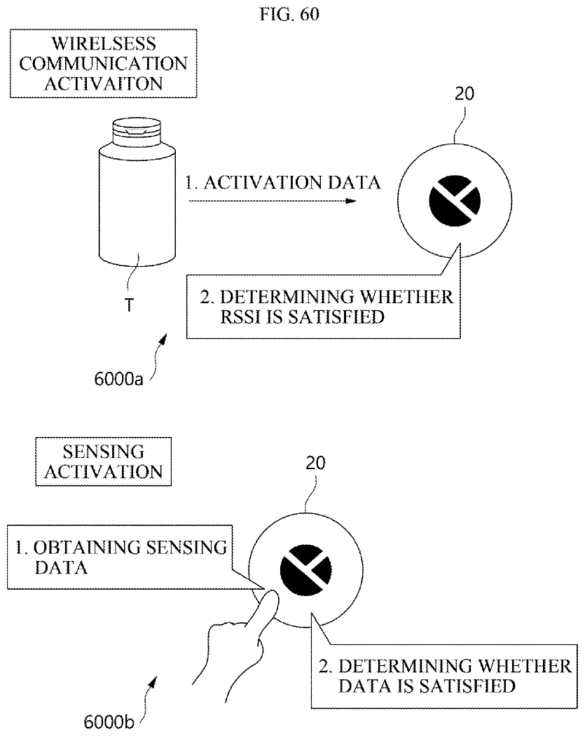

FIG. 60 is a diagram illustrating an example of an activation data for the medication reminder of the health management system 1 according to various embodiments.

According to various embodiments, the health management system 1 (e.g., the server 10) may register the medication condition in operation 5901, and determine whether the medication time is reached in operation 5903. For example, the medication condition may include at least one of the medication time, the medication boost, or the medication quantity (e.g., the number of pills, the injection amount, and the number of injections) for a specific medication target. The local device (e.g., the wearable device 20, the user terminal 30) of the health management system 1 may acquire and store the medication condition for a specific medication target of the user, and transmit the medication condition to the server 10 so that the medication condition is also stored in the form associated with the user in the server 10. After the registration of the medication condition, the health management system 1 (e.g., the server 10, the wearable device 20, the user terminal 30) may determine whether the medication time is reached based on the registered medication condition.

According to various embodiments, the health management system 1 may automatically register a medication condition. For example, when a medication target is registered by the user through a local device (e.g., the wearable device 20 or the user terminal 30), the server 10 may automatically register a medication condition corresponding to the medication target. For example, in the case of a specific type of pill, a medication cycle and/or a medication number of the pill predetermined may be registered as a medication condition.

According to various embodiments, the health management system 1 may manually register a medication condition. As described above, the user may not only input by hand but also may register based on information identified based on a capturing of a prescription using a local device (e.g., the wearable device 20 or the user terminal 30).

According to various embodiments, when the medication time is reached (5903-Y), the health management system 1 (e.g., the wearable device 20 or the user terminal 30) may provide a first alarm for reminding a medication in operation 5905. For example, the first alarm may be provided in a visual, audible, and/or tactile form. For example, the local device (e.g., the wearable device 20 or the user terminal 30) may display a pop-up message for reminding the medication on the display and/or output vibration (or sound). The provision of the first alarm may be maintained for a specific time and/or performed at a specified cycle, but is not limited to the examples described.

According to various embodiments, the health management system 1 (e.g., the wearable device 20 or the user terminal 30) may determine whether to acquire activation data in operation 5907 and continue to provide the first alarm in operation 5909 when the activation data is not acquired (5905-N). For example, when the activation data is not received by the wearable device 20, the wearable device 20 may continue to provide the first alarm and/or may inform the state of un-reception of the activation data from the wearable device 20 to the user terminal 30 to continue to provide the first alarm. The health management system 1 (e.g., the wearable device 20) may determine whether handwriting input is performed in operation 5909, and when the handwriting input is performed (5909-Y), record a medication and not grant an authentication mark in operation 5911, and terminate the first alarm in operation 5913. For example, the handwriting input may be performed as described above, and when information on the medication is input by the handwriting input, the provision of the first alarm may be terminated.

According to various embodiments, the health management system 1 (e.g., the wearable device 20) may determine whether the activation condition is satisfied in operation 5917 when the activation data is acquired (5905-Y), and perform capturing in operation 5919 when the activation condition is satisfied (5917-Y). Referring to FIG. 60, the determination of whether the activation condition is satisfied may include a determination of whether the RSSI of the activation data satisfies a specific condition (e.g., above a threshold) as shown in 6000a of FIG. 60 and/or a determination of whether the user input satisfies a specific condition as shown in 6000b of FIG. 60.

According to various embodiments, the health management system 1 (e.g., the server 10) may perform a medication determination based on the artificial intelligence model in operation 5921, and determine whether the reliability of the medication determination result information exceeds the threshold value (c) in operation 5923. According to various embodiments, the health management system 1 (e.g., the server 10) may assign a medication record and an authentication mark in operation 5923 when the reliability exceeds the threshold value (c) (5921-Y), and control the first alarm to be terminated in operation 5913.

According to various embodiments, the health management system 1 (e.g., the server 10) may provide a second alarm in operation 5925 when the reliability does not exceed the threshold value (e) (5921-N), obtain a handwritten medication record based on a designated template in operation 5927, perform a medication record in operation 5929 but not assign an authentication mark, and control the first alarm to be terminated in operation 5913. Since the second alarm is an alarm described in “5.1.1 medication record and alarm” described above, redundant descriptions are omitted.

### 5.1.3. Multiple Medication Prevention Alarm

According to various embodiments, the health management system 1 may quickly provide an alarm so that the medication performed by the user is not performed once more.

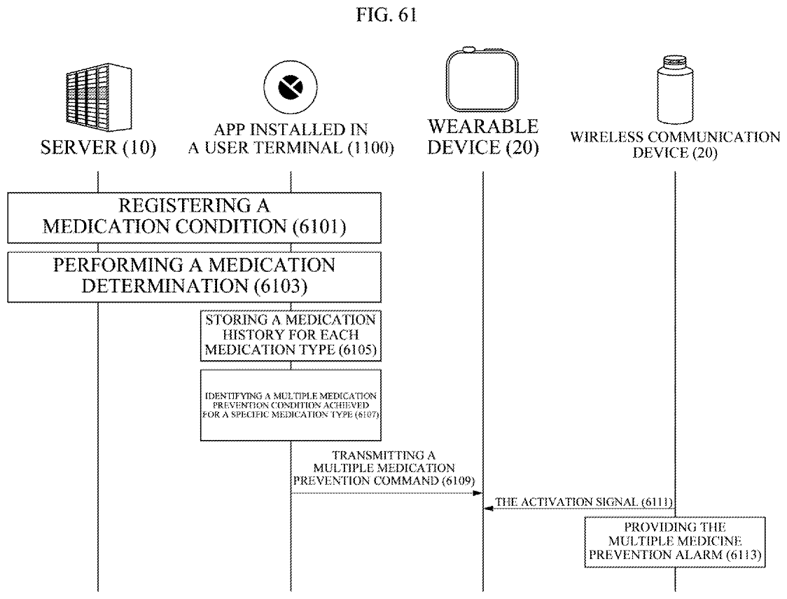

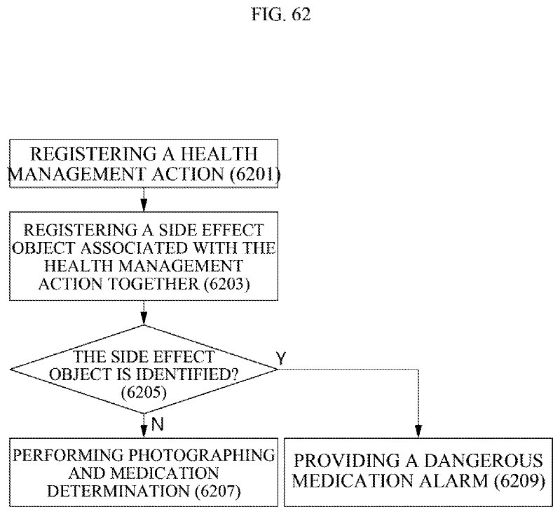

FIG. 61 is a flowchart illustrating an example of an operation for multiple medication alarm of the health management system 1 according to various embodiments. Operations may be performed regardless of the order of the operations shown and/or described, and more operations may be performed and/or fewer operations may be performed.

According to various embodiments, the health management system 1 (e.g., the server 10 and the user terminal 30) may register a medication condition in operation 6101, and perform a medication determination in operation 6103.

According to various embodiments, the health management system 1 (e.g., the server 10 and the user terminal 30) may store a medication history for each medication type in operation 6105, identify a multiple medication prevention condition achieved for a specific medication type in operation 6107, and transmit a multiple medication prevention command to the wearable device 20 in operation 6109. For example, the server 10 may identify the multiple medication prevention condition achieved based on determining whether the medication condition of the specific medication is achieved. For example, the server 10 may identify that the multiple medication prevention condition is achieved when it is determined that the specific medication has been performed at a specific time point at which the specific medication should be performed. For another example, the server 10 may identify that the multiple medication prevention condition is achieved when it is determined that the number of times of the specific medication has been performed.

According to various embodiments, when the activation signal is received in operation 6111, the health management system 1 (e.g., the wearable device 20) may provide the overlapping medicine prevention alarm in operation 6113. For example, the wearable device may provide the overlapping medicine prevention alarm when the activation signal is received based on receiving the overlapping medicine command for a specific medicine. In addition, the wearable device 20 may refrain from activating the camera along with the multiple medication prevention alarm.

According to various embodiments, the wearable device 20 may acquire identification information associated with the specific medicine included in the received activation signal as at least a part of the operation of providing the overlapping medicine alarm, and determine that the multiple medication prevention should be performed based on comparing the identification information with the medicine target where the overlapping medicine prevention condition is achieved. However, the examples are not limited, and may be controlled to provide the multiple medication prevention alarm when the activation signal is simply received.

### 5.1.4. Risk Medication Alarm

According to various embodiments, when it is determined that a user's dangerous medications are performed, the health management system 1 may quickly provide an alarm for them. F

IG 62 is a flowchart illustrating an example of an operation for an alarm of a dangerous medications of the health management system 1 according to various embodiments. Operations may be performed regardless of the order of the operations shown and/or described, and more operations may be performed and/or fewer operations may be performed.

According to various embodiments, the health management system 1 (e.g., the server 10) may register a health management action in operation 6201 and register a side effect object associated with the health management action together in operation 6203. For example, when the performance of a specific medication object is registered, the server 10 may register a medication object in a side effect relationship with the specific medication object.

According to various embodiments, the health management system 1 may determine whether the side effect object is identified in operation 6206, when the side effect object is not identified (6206-N), perform photographing and medication determination in operation 6207, and when the side effect object is identified (6206-Y), provide a dangerous medication alarm in operation 6209. When the medication object in the side effect relationship is identified based on the image captured by the wearable device 20, the server 10 may control the local device (e.g., the wearable device 20, and/or the user terminal 30) to provide the dangerous medication alarm.

### 5.2. Life Log Association

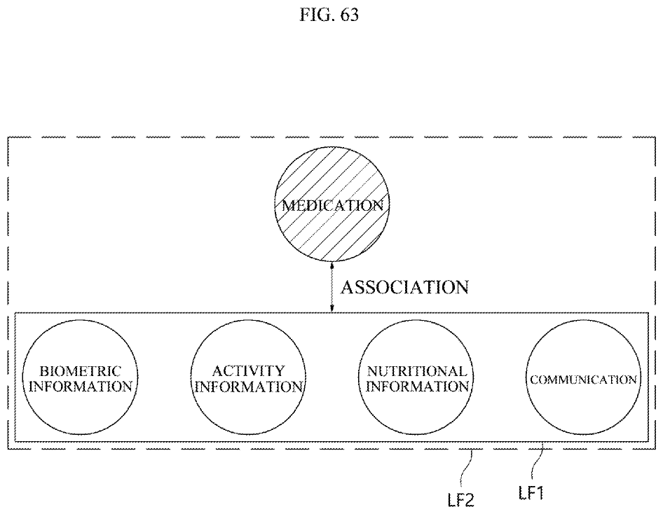

FIG. 63 is a diagram illustrating the operation of linking the life log of the health management system 1 according to various embodiments.

According to various embodiments, the health management system 1 may be implemented to provide a service that linked various kinds of life logs (LF1) (e.g., biometric information, activity information, nutritional information, communication) acquired by sensors of the existing wearable device 20 together with the medication behavior (LF2) identified by the health management system 1. Accordingly, the medication behavior may be extended to the management target of the life log.

In this case, referring to “3. add-on wearable device”, the health management behavior (LF2) may be identified by the first wearable device 2000a and the life log (LF1) may be identified by the second wearable device 2000b, but not limited to the examples described, but may all be identified by a single wearable device 20.

### 5.2.1. Examples of Life Log

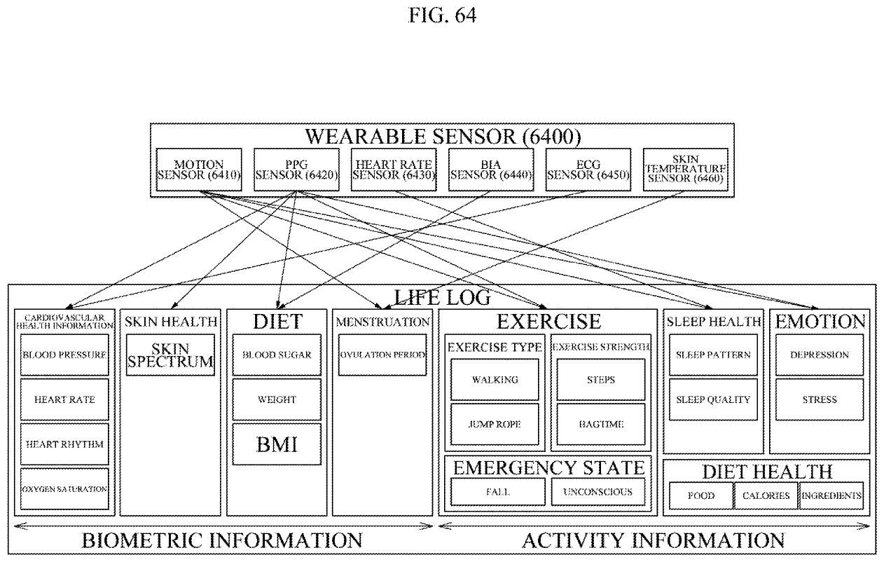

FIG. 64 is a diagram illustrating an example of a life log (LF1) that can be identified using a sensor 6400 of a wearable device 20 according to various embodiments.

According to various embodiments, referring to FIG. 64, the sensor 6400 of the wearable device 20 may include at least one of a motion sensor, a PPG sensor, a heart rate sensor, a BIA sensor, an ECG sensor, or a skin temperature sensor, and is not limited to the illustrated and/or described examples, and may include more sensors and/or fewer sensors. Hereinafter, an example of a life log (LF) obtained based on a value identified using the sensor 6400 will be described according to various embodiments.

According to various embodiments, referring to FIG. 64, the wearable device 20 may obtain information about cardiovascular health such as blood pressure, heart rate, heart rhythm, and oxygen saturation and/or diet-related information such as blood sugar, body weight, and BMI, based on at least one of a PPG value identified using the PPG sensor 6420 or a BIA value identified using the BIA sensor.

According to various embodiments, referring to FIG. 64, the wearable device 20 may obtain information about skin health such as skin spectrum based on a PPG value identified using the PPG sensor 6420.

According to various embodiments, referring to FIG. 64, the wearable device 20 may obtain physiological information such as a ovulation period based on a PPG value identified using the PPG sensor 6420 or a skin temperature value identified using the skin temperature sensor 6460.

According to various embodiments, referring to FIG. 64, the wearable device 20 may obtain information about exercise types and/or exercise strength based on at least one of a PPG value identified using the PPG sensor 6420 or a motion value identified using the motion sensor 6410.

According to various embodiments, referring to FIG. 64, the wearable device 20 may acquire information about sleep health such as sleep pattern, sleep quality, etc. and/or information about emotion such as depression, stress, etc. based on at least one of a motion value identified using the motion sensor 6410 or a heart rate value identified using the heart rate sensor 6430.

### 5.2.2. Medication Correlation Providing Operation

According to various embodiments, the health management system 1 (e.g., the server 10) may obtain a direct correlation between the lifelog (e.g., the lifelog LF of FIG. 52) identified using the wearable device 20 and the result information of the judgment of the medication, and provide information on the obtained correlation.

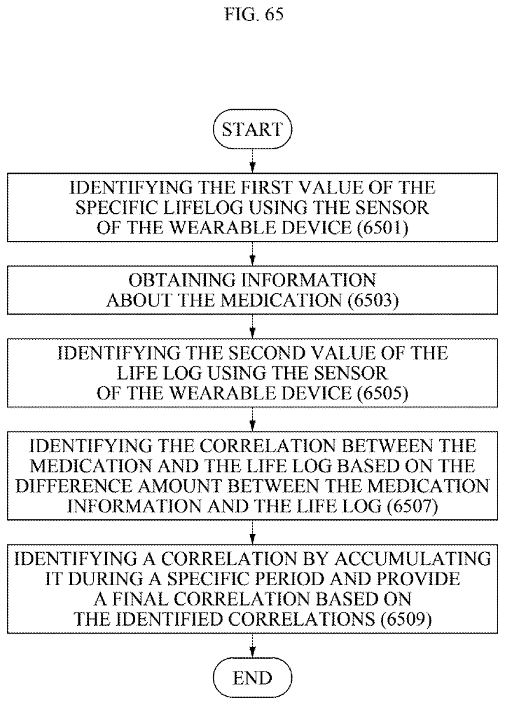

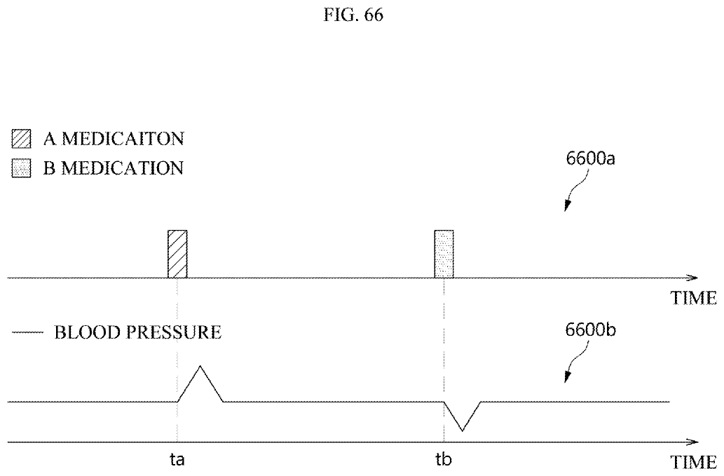

FIG. 65 is a flowchart illustrating an example of an operation of providing medication correlation of the health management system 1 according to various embodiments. Operations may be performed regardless of the order of the operations shown and/or described, and more operations may be performed and/or fewer operations may be performed. Hereinafter, FIG. 65 will be further described with reference to FIG. 66.

FIG. 66 is a diagram illustrating an example of an operation for identifying medication correlation of the health management system 1 according to various embodiments. It will be apparent to those skilled in the art that the 6600a of FIG. 66 may represent the medication target performed over time, and the 6600b of FIG. 66 may represent the value of the specific lifelog (e.g., blood pressure) measured over time, but the medication correlation measurement operation for various types of lifelogs may be performed without being limited to the illustrated example.

According to various embodiments, the health management system 1 (e.g., the server 10) may identify the first value of the specific lifelog using the sensor 6400 of the wearable device 20 in operation 6501. For example, referring to FIG. 54, the server 10 may obtain the first value of the specific lifelog (e.g., blood pressure) identified using the sensor 6400 of the wearable device 20 from the wearable device 20 before the medication ta and th of the user.

According to various embodiments, the health management system 1 (e.g., the server 10) may obtain information about the medication in operation 6503. For example, referring to FIG. 54, the server 10 may receive the medication performance image received from the wearable device 20 at the time of ta and th of the medication performance of the user, and based on the analysis of the medication performance image, the server 10 may identify the information (e.g., the type of medication, the number of times of medication) about the medication together with the medication determination result information.

According to various embodiments, the health management system 1 (e.g., the server 10) may identify the second value of the life log using the sensor 6400 of the wearable device in operation 6505. For example, referring to FIG. 54, the server 10 may obtain the second value of the identified specific life log (e.g., blood pressure) from the wearable device 20 after the time of ta and th of the medication performance of the user using the sensor 6400 of the wearable device 20.

According to various embodiments, the health management system 1 (e.g., the server 10) may identify the correlation between the medication and the life log based on the difference amount between the medication information and the life log in operation 6507. For example, the server 10 may identify the difference value between the first value and the second value for the specific life log, and may measure the correlation between the identified difference value and the information (e.g., the type of medication (or medication target), the amount of medication) about the medication. For example, referring to Equation 1 below, the server 10 may measure the correlation between the specific medication type and the specific life log based on the division of the difference value (d) by the medication amount (m), but is not limited to the examples described, and when the medication amount is not identified, the result value of reflecting a predetermined coefficient in the difference value may be identified as the medication correlation for the specific medication type.

$$Correlation between the specific medication type and the specific life log = the difference value ( d ) / the medication amount ( m ) \qquad [ Equation 1 ]$$

Based on the medication correlation, when the user performs a particular type of medication later, the value of the particular type of life log (e.g., blood pressure) may be predicted after medication performance based on the medication correlation. In this case, as shown in FIG. 66, when the second value is greater than the first value, the particular type of medication (e.g., A medication) may be identified as having a medication correlation in the first direction (e.g., positive) with respect to the particular life log (e.g., blood pressure), and when the second value is less than the first value, the particular type of medication (e.g., B medication) may be identified as having a medication correlation in the second direction (e.g., negative) with respect to the particular life log (e.g., blood pressure).

Meanwhile, the server 10 may further identify the medication amount for the medication object as at least a part of the operation of identifying the medication correlation between the medication object (e.g., the type of medication) and the life log. In other words, the server 10 may calculate the medication correlation between the life log and the medication object for each medication amount of the medication object. The medication correlation may be differently calculated according to the medication amount, and accordingly, the medication correlation between the particular medication and the life log may have a positive value or a negative value.

According to various embodiments, the wearable device 20 may be configured to sense the first life log using at least one sensor at the time when the activation signal is received based on the reception of the activation signal, and to sense the second life log using at least one sensor after a pre-set time elapses from the time when the activation signal is received (or the time when the image capturing by the camera is terminated).

According to various embodiments, the health management system 1 (e.g., the server 10) may be implemented to measure a plurality of medication correlations during a pre-set period of time and to identify a final medication correlation that may become a representative value based on the measured plurality of medication correlations. For example, the pre-set period of time may be determined by the user or may be preset by the server 10. The pre-set period of time may be determined according to the type of medication to be measured and/or the type of life log. The server 10 may calculate an average of values of the plurality of medication correlations collected during the pre-set period of time or may calculate a representative value for the plurality of medication correlations based on various types of algorithms that are not limited to the examples described. Accordingly, the personalized medication correlation for the user of the wearable device 20 may be identified. That is, the medication correlation of the first user and the medication correlation of the second user calculated by the server 10 may be calculated differently.

According to various embodiments, in operation 6509, the health management system 1 (e.g., the server 10) may identify a correlation by accumulating it during a specific period and provide a final correlation based on the identified correlations. For example, the server 10 may be implemented to measure a plurality of medication correlations during a pre-set period and to identify the final medication correlation as a representative value based on the measured plurality of medication correlations. For example, the pre-set period may be determined by the user or may be preset by the server 10. The pre-set period may be determined according to the type of medication to be measured and/or the type of lifelog. The server 10 may calculate an average of values of the plurality of medication correlations collected during the pre-set period or may calculate a representative value for the plurality of medication correlations based on various types of algorithms that are not limited to the examples described. Accordingly, the personalized medication correlation for the user of the wearable device 20 may be identified. That is, the medication correlation of the user of the first wearable device and the medication correlation of the user of the second wearable device calculated by the server 10 may be calculated differently.

### 5.2.3. Chain Correlation Providing Operation

According to various embodiments, the health management system 1 (e.g., the server 10) may obtain a first correlation between different lifelogs (e.g., the lifelog LF of FIG. 52) identified using the wearable device 20 and a second correlation between the lifelog and the result of the judgment of the medication, and provide information on the linkage correlation based on the first correlation and the second correlation.

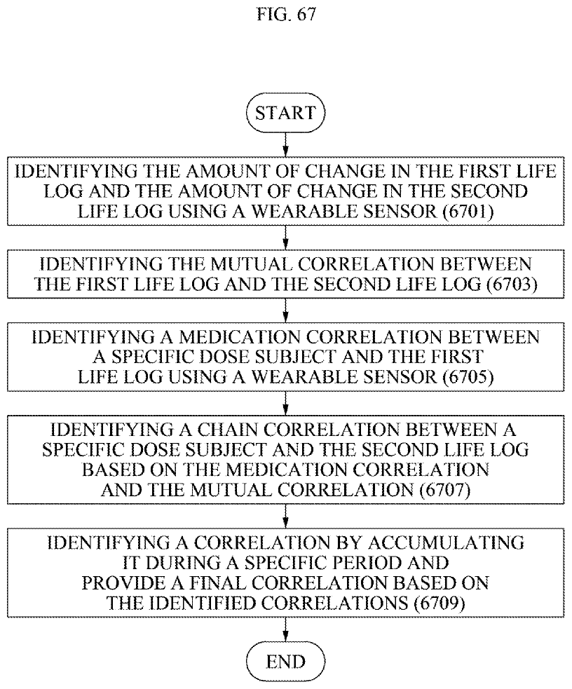

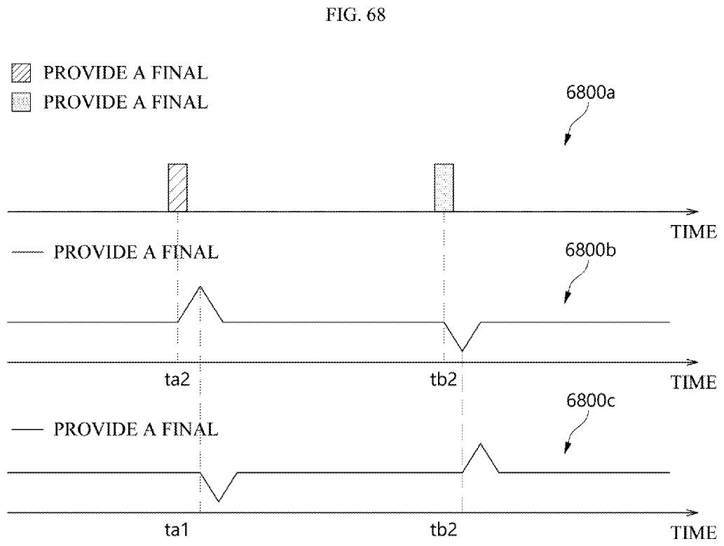

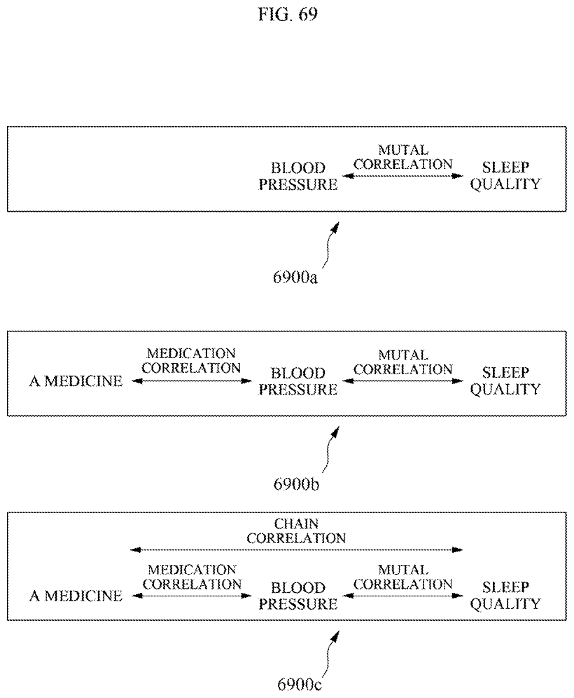

FIG. 67 is a flowchart illustrating an example of an operation of providing the linkage correlation of the health management system 1 according to various embodiments. Operations may be performed regardless of the order of the operations shown and/or described, and more operations may be performed and/or fewer operations may be performed. Hereinafter, FIG. 67 will be further described with reference to FIGS. 68 to 69.

FIG. 68 is a diagram illustrating an example of an operation for identifying the linkage correlation of the health management system 1 according to various embodiments. 6800a of FIG. 68 may represent the medication target performed over time, and 6800b of FIGS. 68 and 609b may represent values of different lifelogs (e.g., blood pressure, sleep quality) measured over time, but they are not limited to the type of the lifelog shown. FIG. 69 is a diagram illustrating an example of the linkage correlation according to various embodiments.

According to various embodiments, the health management system 1 (e.g., the server 10) may identify the amount of change in the first life log and the amount of change in the second life log using a wearable sensor (e.g., the sensor 6400 of the wearable device 20) in operation 6701, and may identify the mutual correlation between the first life log and the second life log in operation 6703. For example, referring to 6900a of FIG. 69, the mutual correlation may indicate a correlation between different types of life logs (e.g., blood pressure and sleep quality). For example, referring to FIG. 68, the server 10 may acquire a plurality of life logs (e.g., blood pressure and sleep quality) based on values identified using at least one sensor 6400 of the wearable device 20. The server 10 may identify the change in the values of the plurality of acquired life logs (e.g., blood pressure and sleep quality), and may identify the mutual correlation based on the change in the identified values. For example, the server 10 may acquire a first value of another second life log (e.g., sleep quality) before the time (ta1, tb1) and a second value of the second life log (e.g., sleep quality) after the time (ta1, tb1) based on the time (ta1, tb1) at which the value of the first life log (e.g., blood pressure) has changed (or changed), and may identify a difference value d1 between the first value and the second value. As shown in Equation 2 below, the server 10 may calculate a correlation between the first life log (e.g., blood pressure) and the second life log (e.g., quality of sleep) based on a difference value d1 of the second life log (e.g., quality of sleep) and the change amount d2 of the first life log (e.g., blood pressure) and.

$$Correlation between the first life log and the second life log = d 1 / d 2 \qquad [ Equation 2 ]$$

Although not limited to that described in Equation 2, Equation 2 may further include a predetermined coefficient, detailed descriptions thereof will be omitted. Meanwhile, the correlation of the first life log with respect to the second life log (e.g., quality of sleep) may be inferred as d2/d1.

According to various embodiments, in operation 6705, the health management system 1 (e.g., the server 10) may identify a medication correlation between a specific dose subject (e.g., A dose, B dose) and the first life log (e.g., blood pressure) using a wearable sensor (e.g., the sensor 6400 of the wearable device 20). For example, referring to FIG. 68, the server 10 may obtain the medication correlation between the dose type (or the dose subject) and the life log described in the medication correlation providing operation “5.2.2. The medication correlation between the dose type (or the dose subject) and the life log may be obtained. Meanwhile, without limitation to the described example, the server 10 may obtain the medication correlation between the dose evaluation result (e.g., A dose, B dose) and the second life log (e.g., quality of sleep).

According to various embodiments, in operation 6707, the health management system 1 (e.g., the server 10) may identify a chain correlation between a specific dose subject and the second life log based on the medication correlation and the mutual correlation. For example, the server 10 may identify the chain correlation between the dose subject and the second life log based on the above-described medication correlation between the dose subject (e.g., A dose, B dose) and the first life log and the mutual correlation between the first life log and the second life log.

According to various embodiments, in operation 6709, the health management system 1 (e.g., the server 10) may identify a correlation by accumulating it during a specific period and provide a final correlation based on the identified correlations. For example, the server 10 may measure a plurality of medication correlations and a plurality of mutual correlations during a pre-set period and identify a plurality of chain correlations based on the measured correlations (e.g., medication correlations, mutual correlations). The server 10 may be implemented to identify the final medication correlation as a representative value among the correlations (e.g., medication correlations, mutual correlations, chain correlations) identified during the pre-set period. Since the operation of identifying the representative value may be performed as in operation 6509 described above, redundant descriptions are omitted.

### 5.2.4. Service Providing Operation Based on Correlation

According to various embodiments, the health management system 1 (e.g., the server 10) may provide at least one service for managing a life log based on a medication based on the above-described correlations (e.g., medication correlation, mutual correlation, and chain correlation).

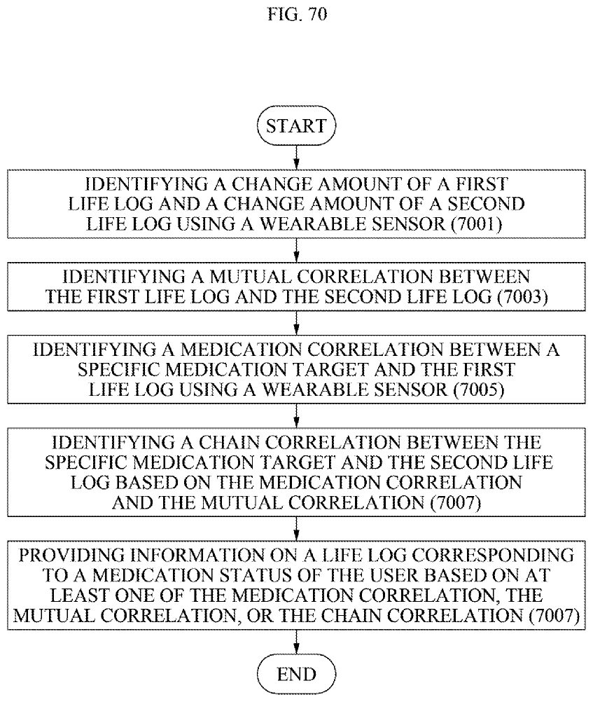

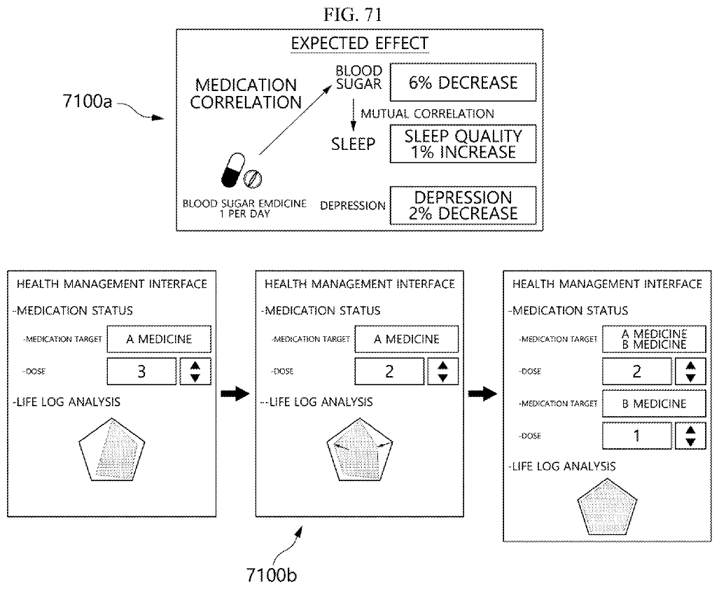

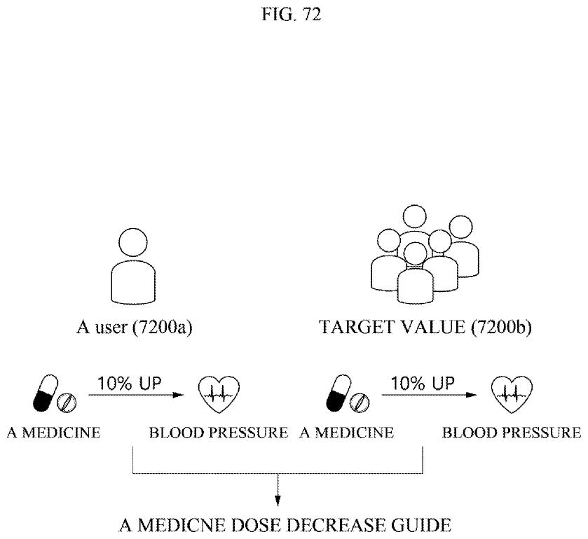

FIG. 70 is a flowchart illustrating an example of an operation of providing a correlation associated with the health management system 1 according to various embodiments. Operations may be performed regardless of the order of the operations shown and/or described, and more operations may be performed and/or fewer operations may be performed. Hereinafter, FIG. 70 will be further described with reference to FIGS. 71 to 72.

FIG. 71 is a diagram illustrating examples of an operation of providing a service for managing a life log of a user based on a correlation of the health management system 1 according to various embodiments. FIG. 72 is a diagram illustrating another example of an operation of providing a service for managing a life log of a user based on a correlation of the health management system 1 according to various embodiments.

According to various embodiments, the health management system (e.g., the server 10) may identify a change amount of a first life log and a change amount of a second life log using a wearable sensor (e.g., the sensor 6400 of the wearable device 20) in operation 7001, identify a mutual correlation between the first life log and the second life log in operation 7003, identify a medication correlation between a specific medication target and the first life log using a wearable sensor (e.g., the sensor 6400 of the wearable device 20) in operation 7005, and identify a chain correlation between the specific medication target and the second life log based on the medication correlation and the mutual correlation in operation 7007.

According to various embodiments, in operation 7009, the health management system (e.g., the server 10) may provide information on a life log corresponding to a medication status of the user based on at least one of the medication correlation, the mutual correlation, or the chain correlation.

In an embodiment, the server 10 may be implemented to provide a change amount of at least one life log expected (or predicted) when a specific medication is performed based on at least one of the above-described correlations (e.g., the medication correlation, the mutual correlation, or the chain correlation). For example, as shown in 7100a of FIG. 71, the server 10 may control an execution screen including the change amounts of the expected life logs when a specific medication (e.g., taking a blood pressure medicine) is displayed on a local device (e.g., the wearable device 20, the user terminal 30). For example, the server 10 may provide an execution screen for receiving information on a specific medication to the local device (e.g., the wearable device 20, the user terminal 30), and receive execution of the specific medication (e.g., a blood pressure medicine) through the execution screen. The server 10 may control an execution screen including a change amount of a first life log (e.g., blood pressure) calculated based on a medication correlation with a first life log (e.g., blood pressure) associated with a specific medication (e.g., taking a blood pressure medicine) and a change amount of a second life log (e.g., quality of sleep) identified based on a mutual correlation between the first life log (e.g., blood pressure) and the second life log (e.g., quality of sleep). Here, the change amount of the second life log (e.g., quality of sleep) may be provided based on a chain correlation calculated between the specific medication and the second life log, without being limited to the described example.

In another embodiment, the server 10 may be implemented to provide the amount of change in the life log based on another medication information (e.g., dose, dose cycle, and the like) input together with the medication target (or medication type). For example, as shown in 7100b of FIG. 71, the server 10 may control an execution screen including the amount of change between a specific medication target (e.g., A medicine) and at least one life log to be displayed on a local device (e.g., the wearable device 20, the user terminal 30). In this case, the server 10 may provide the amount of change in the life log based on the medication correlation between the specific medication target (e.g., A medicine) and at least one life log and the specific medication target (e.g., A medicine). For example, the amount of change in the life log may be proportional to the medication, but it is not limited to the example described, and in the case of the specific medication, the correlation between the life log and the medication correlation may not be proportional as the correlation is set to a value in the opposite direction (e.g., change from a negative value to a positive value). In this case, the server 10 may control the value of the life log that is sequentially changed according to the change in the specific medication target (e.g., A medicine) to be provided on the execution screen. In addition, the server 10 may calculate the amount of change in the life log according to the specific medication target (e.g., A medicine) as well as the amount of change in the life log according to another additional medication target (e.g., B medicine), and provide a complex amount of change in the life log.

In another embodiment, the server 10 may identify a target dose for a specific medication set based on accumulated correlations (e.g., medication correlation, mutual correlation, and chain correlation) for each of the plurality of users, and provide the identified target dose to the user. For example, as shown in 7200b of FIG. 72, the server 10 may identify an average medication correlation (e.g., 8% blood pressure increase at dose) based on medication correlations between a specific dose (e.g., A medicine) and a life log (e.g., blood pressure) for each of the plurality of users. As shown in 7200a of FIG. 72, the server 10 may provide a dose control guide when there is a difference between a medication correlation and an average medication correlation between a specific dose (e.g., A medicine) and a life log (e.g., blood pressure) of a specific user (e.g., A user). For example, when the medication correlation of the specific user (e.g., A user) has a higher positive value than the average medication correlation, a dose reduction guide may be provided, and when the medication correlation of the specific user (e.g., A user) has a lower positive value than the average medication correlation, a dose increase guide may be provided. It is obvious to those skilled in the art that when the medication correlation is all negative values, the opposite guide may be provided.

### 5.2.5. Digital Medicine

According to various embodiments, the health management system 1 may be used as a digital therapeutic agent that determines diseases currently held by a user using the wearable device 20 and provides a software-based treatment solution for the determined diseases.

As a disease that can be determined through the health management system 1, for example, Parkinson's disease, insomnia, and schizophrenia may be present. Hereinafter, a method of utilizing the above-described diseases as a digital therapeutic agent will be described as an example. However, the above-listed diseases are exemplary, and are not necessarily limited thereto, and all diseases that can be determined through the wearable device may be targeted.

First, Parkinson's disease is characterized by a decrease in cognitive ability and problem-solving ability, and a handshake symptom appears along with hallucination symptoms, insomnia is characterized by a lack of sleep or a deep sleep caused by excessive stress, and schizophrenia is a biological disease caused by abnormality in the brain, and show symptoms of hearing loss, delusions, and language disabilities. That is, since each disease exhibits various symptoms and characteristics for each disease, it is possible to determine the disease using these characteristics.

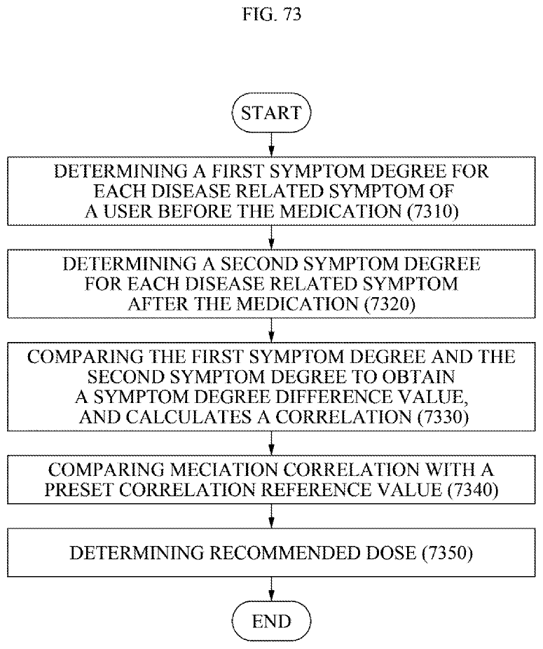

FIG. 73 is a flowchart illustrating a method of operating a digital therapeutic agent in a health management system according to some embodiments of the present disclosure. Referring to FIG. 73, the health management system I determines a first symptom degree for each disease related symptom of a user before the medication (S7310), and determines a second symptom degree for each disease related symptom after the medication (S7320). Here, each of the first symptom degree and the second symptom degree may be a symptom degree measured based on a hand image of a user acquired using a camera of the wearable device 20, and the related symptom may include a representative symptom that is a representative feature appearing in each of various diseases and side effects that are additional symptoms separately represented from the representative symptom.

Thereafter, the health management system 1 compares the first symptom degree and the second symptom degree to obtain a symptom degree difference value, and calculates a correlation (S7330). The calculated correlation is compared with a preset correlation reference value (S7340), and the health management system 1 determines a recommended medication amount according to the comparison result (S7350) and guides the determined recommended medication amount to the user, thereby providing a digital treatment solution.

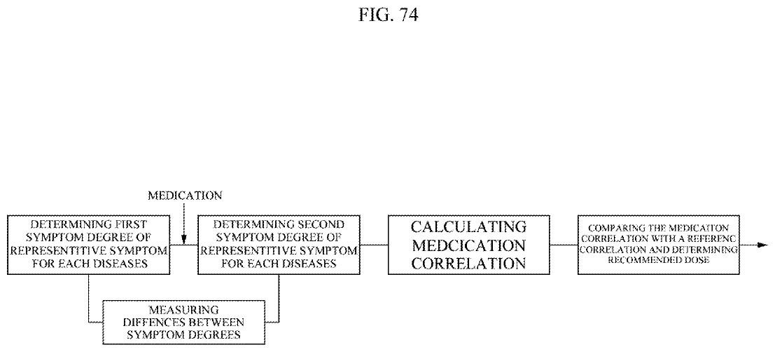

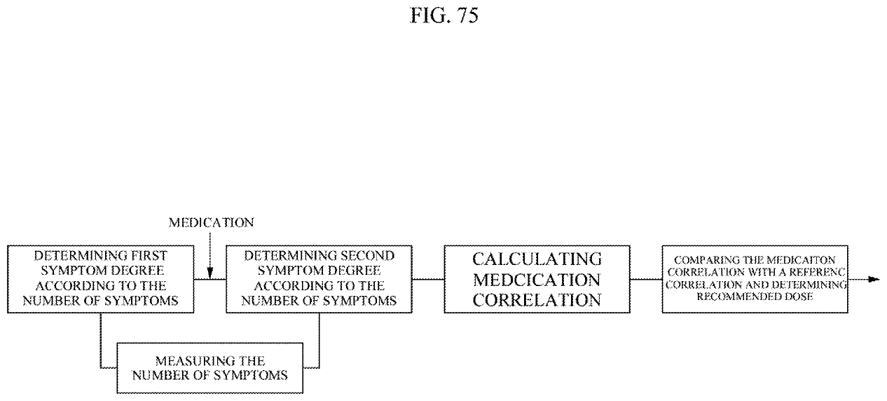

FIG. 74 is a diagram illustrating a method of providing a medication amount according to a medication after the symptom degree in some embodiments of the present disclosure, and FIG. 75 is a diagram illustrating a method of providing a medication amount according to a medication after the symptom degree in other embodiments of the present disclosure.

First, referring to FIG. 74, the health management system I determines a first symptom degree for each disease representative symptom, and determines a second symptom degree for a medication after the representative symptom. Here, the expressed representative symptoms are medical and physical features frequently expressed in the corresponding disease, and as described above, may be different for each disease, and the first symptom degree and the second symptom degree for the representative symptoms may be different by drugs absorbed by the body after the medication is performed.

For example, in the case of Parkinson's disease, since hand tremor is a representative symptom, the degree of the representative symptom may be the number of hand tremors occurring during a predetermined time (e.g., 120 times per minute), and in the case of insomnia, since the decline in heart beat caused by the shortage of sleep is a representative symptom, the degree of the representative symptom may be the heart rate during a predetermined time, and in the case of schizophrenia, since language disorder is a representative symptom, the degree of the representative symptom may be the response rate (fire rate) to a personalized question.

Thereafter, the health management system 1 calculates the first medication correlation according to the ratio of the difference between the first degree of symptom and the second degree of symptom and the dose. The first medication correlation is the correlation between the dose of a specific medicine and the symptoms caused by the dose, and may be calculated by [Equation 3] below.

$$The first medication correlation = {k}_{1} \vert \frac{the second degree of symptom - the first degree of symptom}{dose} \vert \qquad [ Equation 3 ]$$

For example, if the first symptom, which is the number of handshakes in Parkinson's disease, is 120 times per minute, the second symptom is 60 times per minute, and the dose is 60 mg, the first medication correlation may be 1 (assuming k1 is a proportional constant and k1=1). Here, the higher the difference between the first symptom degree and the second symptom degree, the higher the first medication correlation, and the lower the dose. In other words, the greater the difference between the first symptom and the second symptom, the higher the medication effect, and it means that the smaller the dosage amount has a higher effect even with a smaller drug dose. Accordingly, the first medication correlation is proportional to the difference between the first symptom and the second symptom, and is inversely proportional to the dose.

The health management system 1 determines the recommended dose by comparing the calculated first medication correlation with a preset first reference correlation (w). Here, the preset first reference correlation (w) may be an average medication correlation of patients with a corresponding disease, or may be a randomly set target medication correlation.

In various embodiments, the health management system 1 may determine a dose that is increased than the current dose as the recommended dose if the calculated first medication correlation is smaller than the first reference correlation (w), determine a dose that is the same as the current dose as the recommended dose if the calculated first medication correlation is the same as the first reference correlation (w), and determine a dose that is decreased than the current dose as the recommended dose if the calculated first medication correlation is greater than the first reference correlation (w).

In addition, in various embodiments, the health management system 1 may specifically determine an increase or decrease in the recommended dose based on the first medication correlation, the preset first reference correlation (w), and the current dose.

The increase or decrease in the recommended dose may be calculated by the following Equation (4).

$$The increase or decrease in the recommended dose = {k}_{2} \vert \frac{w - the medication correlation}{the current dose} \vert \qquad [ Equation 4 ]$$

For example, when the first reference correlation (w) preset in Parkinson's disease is 15, the first medication correlation of the calculated user is 10, and the current dose is 5, (assuming that k2 is a proportional constant and k2=1), the increase/decrease amount may be (15−10)/5=1. Therefore, the final recommended dose may be 6 with 1 increased from the current dose 5.

As described above, the health management system 1 of the present disclosure may provide a digital treatment solution for a specific disease by individually determining the symptom degree of the disease of the user, calculating a first medication correlation, and providing a recommended dose.

Meanwhile, in various embodiments, the symptom degree may be determined based on the number of symptoms expressed for each disease. Referring to FIG. 75, symptoms that may occur in Parkinson's disease include tremor, stiffness, slowing (slow movement), a seismic, autonomous nervous system abnormalities, and sensory abnormalities, and the symptom degree may be determined based on the number of expressed symptoms.

The occurrence of symptoms may be determined by comparing each symptom with a reference value, and for example, in the case of tremor, if it is equal to or higher than 7 Hz, it may be determined to develop tremor symptoms, in the case of stiffness, if there is no movement for a predetermined time (e.g., 10 seconds), it may be determined to develop stiffness, in the case of slow movement, if it is a speaking speed or walking speed equal to or lower than a predetermined speed, it may be determined to develop slow movement symptoms, and in the case of a posture or higher, it may be determined to develop a posture or higher symptoms based on the angle of the head or the waist.

The health management system 1 determines the first symptom by counting the number of pre-medication developed symptoms, and determines the second symptom by counting the number of post-medication developed symptoms. At this time, the symptom may be determined by applying a weight to the representative symptoms for each disease (e.g., counting twice when the representative symptoms are developed).

The health management system 1 calculates a second medication correlation, which is a correlation between a dose and the number of expressed symptoms, using the first symptom degree and the second symptom degree. The second medication correlation may be calculated based on the ratio of the first symptom degree and the second symptom degree, and specifically, may be calculated by Equation (5) below.

$$The second medication correlation = {k}_{3} \vert \frac{the first symptom degree}{the second symptom degree} \vert \qquad [ Equation 5 ]$$

Here, k3 means a proportional constant, and the second medication correlation has a high value if the number of second expression symptoms is small compared to the number of first expression symptoms, and thus the higher the value of the second medication correlation, the better the efficacy of the corresponding dose.

Thereafter, the health management system 1 compares the calculated second medication correlation with the preset second reference correlation (r) to determine the recommended dose. The preset second reference correlation (r) may be arbitrarily set target reference ratio.

For example, if the second medication correlation is smaller than the preset second reference correlation, it means that the efficacy of the drug in the dose is small or not active, and thus the health management system 1 may determine the recommended dose by increasing the dose cycle, the dose capacity, and the number of doses. On the other hand, if the second medication correlation is significantly greater than the preset second reference correlation, it may be determined that the recommendation is replaced with a drug different from the drug currently in use.

If the second medication correlation is the same as the preset second reference correlation, it means that the efficacy of the drug in the dose is properly active, and thus the health management system 1 may determine the recommended dose by maintaining the dose cycle, the dose capacity, and the number of doses.

If the second medication correlation is greater than the preset second reference correlation, it means that the efficacy of the drug in the dose is large, and thus the health management system 1 may determine the recommended dose by decreasing the dose cycle, the dose capacity, and the number of doses.

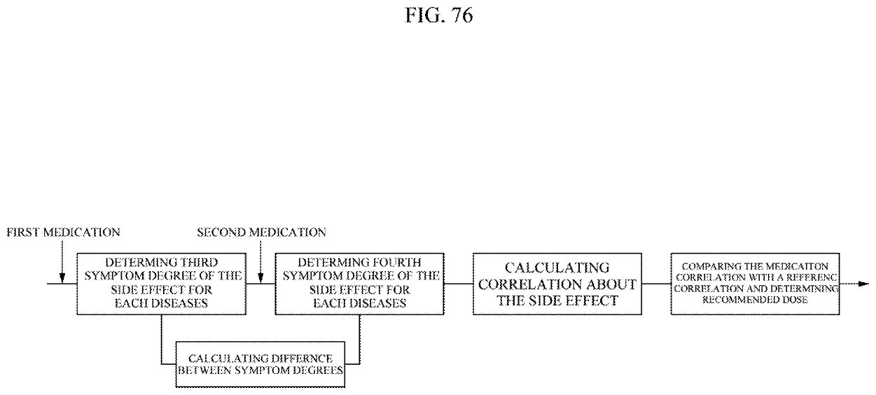

FIG. 76 is a diagram illustrating a method of providing a dose according to side effects after a dose according to some embodiments of the present disclosure.

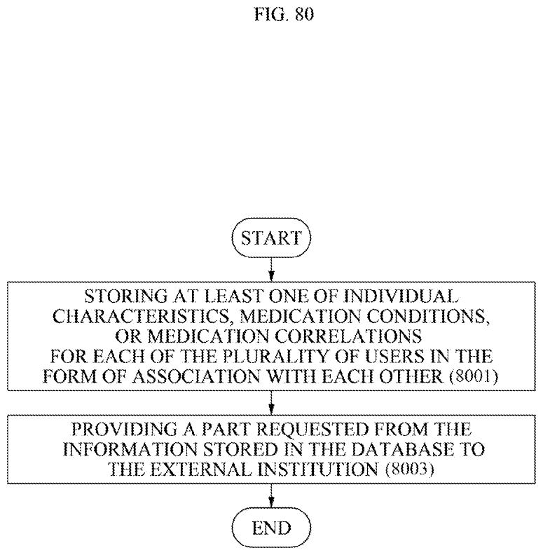

In various embodiments of the present disclosure, as shown in FIG. 80, the symptom degree may be determined based on the degree of side effects occurring to a user for each disease. For example, side effects that may occur in Parkinson's disease include dyspepsia, side effects that may occur in insomnia include memory impairment, and side effects that may occur in schizophrenia include sleepiness or weight gain. The symptom degree may be determined based on at least one of the degree of occurrence of side effects, the number of occurrences and the duration of side effects, and the health management system 1 may determine the symptom degree for each disease by including various sensors (e.g., body fat detection sensors, sensor capsules, sleepiness detection sensors, heart rate detection sensors, and the like) that may sense various symptoms and output them as a quantitative value.

The health management system I determines the side effect symptoms for each taking action through a plurality of doses of a user. In detail, the health management system 1 may determine a third symptom degree, which is the degree of occurrence of side effects for each disease according to the first dose after the first dose, and determine a fourth symptom degree, which is the degree of occurrence of side effects for each disease according to the second dose after the second dose. In this case, the second dose time point may be a time point after a considerable time has elapsed so that the efficacy of the first dose is sufficiently expressed after the first dose.

The health management system 1 calculates a third medication correlation, which is a correlation between a dose and side effects of a specific drug, based on the third symptom degree, the fourth symptom degree, the first dose and the second dose. Specifically, the third medication correlation may be calculated by Equation 6 below.

$$The third medication correlation = {k}_{4} \vert \frac{the fourth symptom degree - the third symptom degree}{the second dose - the first dose} \vert \qquad [ Equation 6 ]$$

For example, in schizophrenia, if the third symptom degree, which is the heart rate, is 110 times per minute, the fourth symptom degree is 130 times per minute, and the first dose is 20 mg and the second dose is 30 mg, the third medication correlation may be 2 (k4 is a proportional constant, assuming k4=1). Here, the third medication correlation has a higher value as the difference between the third symptom degree and the fourth symptom degree is larger, and the higher value as the difference between the first dose and the second dose is smaller. That is, the third medication correlation is higher as the difference between the third symptom degree and the fourth symptom degree is larger, and the third medication correlation is higher as the difference between the first dose and the second dose is smaller, so that the possibility of a side effect caused by the corresponding drug dose is high.

The health management system 1 determines the recommended dose by comparing the calculated third medication correlation with the preset third reference correlation (u). Here, the third reference correlation (u) may be a medication correlation including the maximum allowable side effect symptom degree of the corresponding disease, or a randomly set target side effect correlation.

In an embodiment, the health management system 1 may determine the recommended dose by increasing the amount of the total dose based on the calculated third medication correlation larger than the third reference correlation (u), determine the recommended dose by increasing the amount of the total dose based on the calculated third medication correlation equal to the third reference correlation (u), and determine the recommended dose by decreasing the amount of the total dose based on the calculated third medication correlation smaller than the third reference correlation (u).

Meanwhile, in various embodiments, the health management system 1 may calculate the general medication correlation by considering both the amount of symptoms for representative symptoms and the amount of symptoms for side effects for each disease, which are the above embodiments, and determine the recommended dose based on the calculated general medication correlation. The calculation of the general medication correlation and the determination of the recommended dose based on the representative symptoms and the side effect symptoms may be combined in various methods and forms, and detailed descriptions are omitted here.

As described above, the present invention has been described with reference to the illustrated drawings, but the present invention is not limited by the embodiments and drawings disclosed in this specification, and it is obvious that various modifications can be made by those skilled in the art within the scope of the technical idea of the present invention. In addition, even if the effects of the present invention are not explicitly described while describing the embodiments of the present invention, it is natural that the effects predictable by the corresponding configurations should also be recognized.

### 6. Manager Scenario

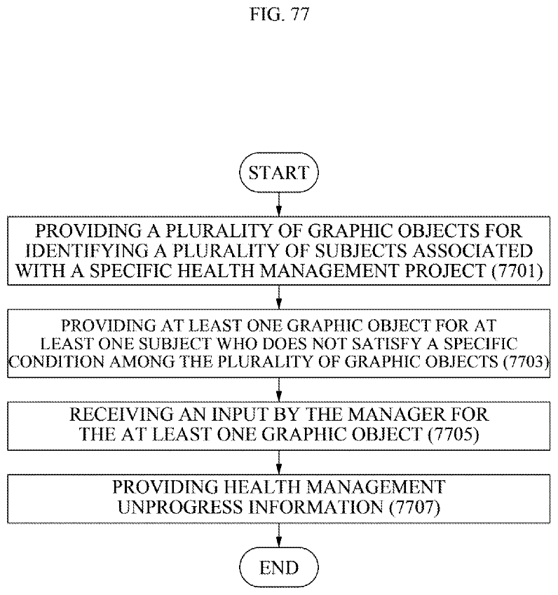

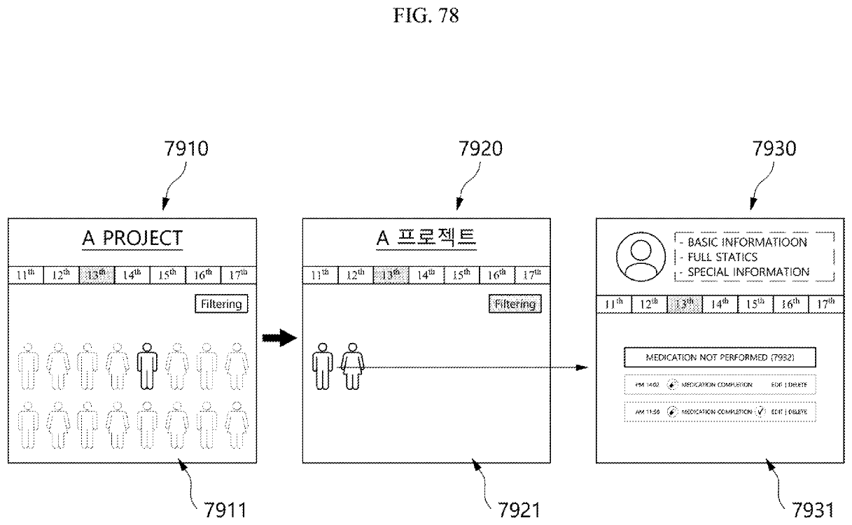

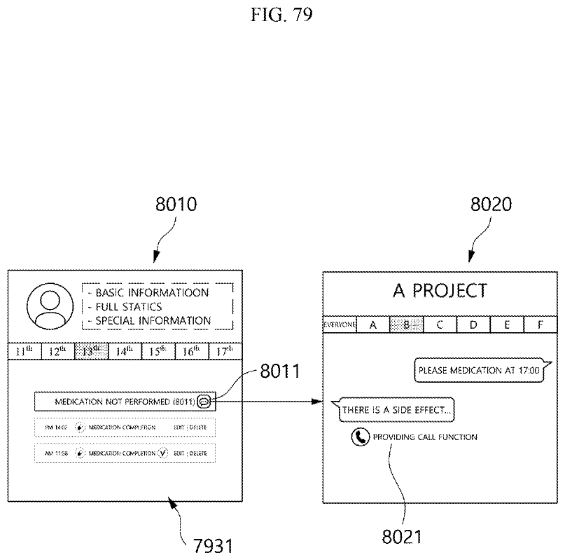

FIG. 77 is a flowchart illustrating an example of an operation of providing an execution screen for a manager for managing a medication of a user of a health management system 1 according to various embodiments. Operations may be performed regardless of the order of the illustrated and/or described operations, and more operations may be performed and/or fewer operations may be performed. Hereinafter, FIG. 77 will be further described with reference to FIGS. 78 to 79.

FIG. 78 is a diagram illustrating an example of an execution screen for a manager for managing a medication of a user provided from a health management system 1 according to various embodiments. FIG. 79 is a diagram illustrating an example of an execution screen for a manager for managing a medication of a user provided from a health management system 1 according to various embodiments.

According to various embodiments, in operation 7701, the health management system 1 (e.g., the server 10) may provide a plurality of graphic objects for identifying a plurality of subjects associated with a specific health management project. For example, when the manager's user terminal 30 for managing users using the server 10 is connected, the server 10 may provide an execution screen for managing a medication of a plurality of users associated with the manager. The manager may be a clinical tester managing medication performers, medical staff performers and family performers, and may be a user managing a medication status of medication performers using the health management system 9. For example, the server may provide a plurality of icons 7811 corresponding to a plurality of medication performers associated with (i.e., managed) the manager to the user terminal 30. The plurality of icons 7811 may be provided in various shapes rather than a human shape, and may be implemented by a menu in a drop-down format.

According to various embodiments, the server 10 may provide a plurality of icons 7811 for each date in the execution screen 7810, but is not limited to the examples described and/or illustrated, and the icons 7811 may be provided for each larger unit of time (e.g., monthly, weekly, and/or smaller unit of time), not for each date.

According to various embodiments, the health management system 1 (e.g., the server 10) may provide at least one graphic object for at least one subject who does not satisfy a specific condition (e.g., a medication condition) among the plurality of graphic objects in operation 7703, and receive an input by the manager for the at least one graphic object in operation 7705. For example, when the filtering function is executed by the manager, the server may filter at least one icon 7821 of medication performers who do not satisfy the specific medication condition among the plurality of icons 7811, and control the execution screen 7820 including the at least one icon 7821 to be provided to the user terminal 30 of the manager. The specific medication condition includes the number of times of medication for each date, a pre-set medication time, and a medication amount, and accordingly, when the number of times of medication designated for a specific date is not satisfied, a medication determination is not performed at a pre-set medication time, or a medication amount is not satisfied, the specific medication condition may be identified as unsatisfied. When the at least one graphic object 7821 is selected, the server 10 may control the execution screen 7830 including the medication determination result information 7831 for a specific user to be provided to the user terminal 30.

According to various embodiments, in operation 7707, the health management system 1 (e.g., the server 10) may provide health management unprogress information. For example, the server 10 may provide medication determination result information that lacks medication conditions on the execution screen 7830 including the medication information 7831, as shown in FIG. 78. For example, as shown in 7832 of FIG. 78, information about a pre-set medication time in which medication determination result information is not generated may be provided. In addition, the medication determination result information may be generated, but the medication conditions are not satisfied (e.g., insufficient medication amount), and/or the medication determination result information having a low reliability may be visually highlighted. Referring to FIG. 79, the server 10 may provide a function for communication with a user who lacks the medication condition. For example, as shown in 7910 of FIG. 79, a graphic object 7911 (e.g., an icon) for activating a communication function may be provided in a form associated with the medication determination result information in which the medication conditions are not satisfied, and when the graphic object 7911 is selected, an execution screen for sending/receiving a message to/from the medication performer may be provided, and/or an icon 7021 for performing an additional call may be provided. In this case, when the graphic object 7911 (e.g., an icon) for activating the communication function is selected, a message including a specific phrase (e.g., a medication query for a time when the medication conditions are not satisfied), and/or a message for activating a specific function (e.g., an alarm) may be automatically provided to the local device (e.g., the wearable device 20 or the user terminal 30) of the medication performer.

### 7. Business Model

### 7.1 Datamining

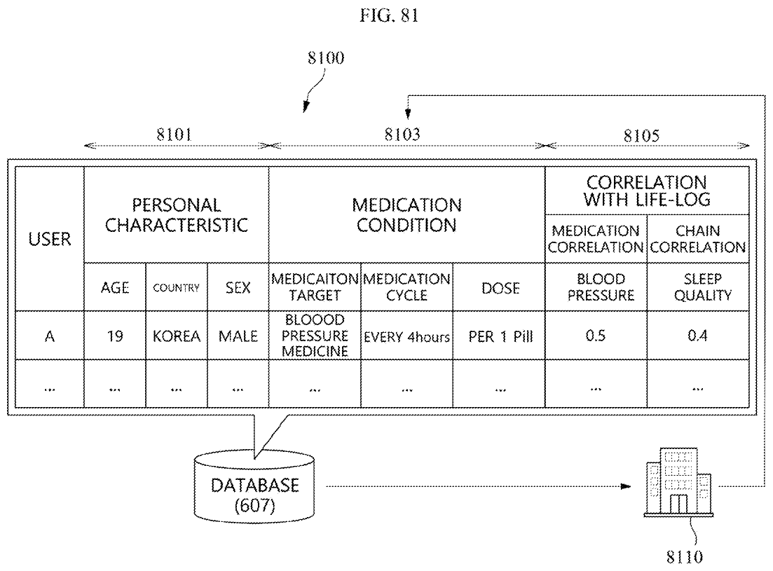

FIG. 80 is a flowchart illustrating an example of an operation for managing information of a health management system 1 and providing information to another external server according to various embodiments. Operations may be performed regardless of the order of the operations shown and/or described, and more operations may be performed and/or fewer operations may be performed. Hereinafter, FIG. 80 will be further described with reference to FIG. 81.

FIG. 81 is a diagram illustrating an example of a data mining and providing method of a health management system 1 according to various embodiments.

According to various embodiments, the health management system 1 (e.g., the server 10) may store at least one of individual characteristics, medication conditions, or medication correlations for each of the plurality of users in the form of association with each other in operation 8001, and provide a part requested from the information stored in the database to the external institution in operation 8003. Referring to FIG. 81, the server 10 may store information 8100 associated with each of the plurality of users using the health management system 1 in the database 507. The information 8100 may include information on individual characteristics 8101, information on medication conditions 8103, and/or information on the life log correlation 8105 in the form of association with the identification information (e.g., name, ID) of a specific user. The respective information 8101, 8103, and 8105 is the same as described above, and thus redundant descriptions are omitted. The server 10 may provide at least a part requested from the external server 8110 from the information 8101, 8103, and 8105 to the external server 8110. The external server 8110 may be a server such as a hospital, a public institution, and a company that needs to analyze the life log.

### 7.2. Medical Incentive

### 7.2.1. Basic Operation

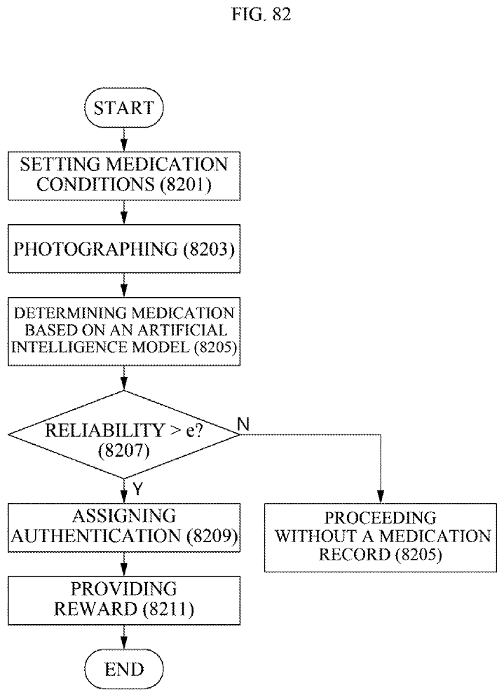

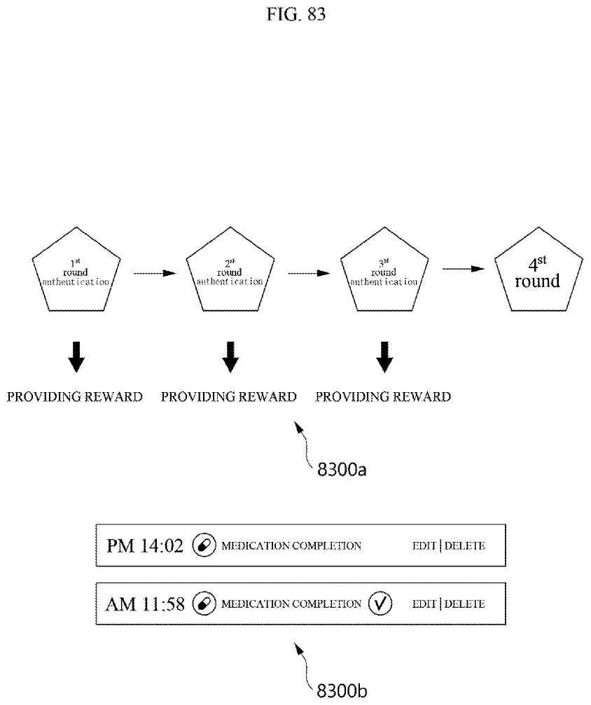

FIG. 82 is a flowchart illustrating an example of an operation for providing medication authentication and reward for a user of the health management system 1 according to various embodiments. Operations may be performed regardless of the order of the illustrated and/or described operations, and more operations may be performed and/or fewer operations may be performed. Hereinafter, FIG. 82 will be further described with reference to FIG. 83.

FIG. 83 is a diagram illustrating an example of medication authentication and reward authorization of the health management system 1 according to various embodiments.

According to various embodiments, the health management system 1 may set medication conditions in operation 8201, and determine medication based on an artificial intelligence model in operation 8205 when photographing is performed in operation 8203, and determine whether the confidence level for the medication determination result is greater than the threshold (e) in operation 8207.

According to various embodiments, when the confidence level is greater than the threshold (c), the health management system 1 may assign authentication in operation 8209 and provide reward in operation 8211. For example, referring to 8300a and 8300b of FIG. 83, when the confidence level for the medication determination result is greater than the threshold, the server 10 may identify that a specific user performed medication management using the health management system 1 and provide reward along with the assignment of medication authentication marks to the specific user. In other words, reward may be provided for the medication determination result to which the above-described medication authentication marks are assigned. The reward may be used to purchase products (e.g., health functional foods) provided by the health management system 1 and/or may be used.

According to various embodiments, when the confidence level is less than the threshold (e), the health management system 1 may proceed without a medication record in operation 8213. Meanwhile, the medication record proceeding without means that the medication information is insufficiently recorded while a record is performed on the medication judgment result, and accordingly, the medication record is performed, and as described above, the medication authentication mark may be unassigned.

Meanwhile, it is obvious to those skilled in the art that the incentive as described above may be granted to various health management actions (e.g., diet management) other than medication management.

### 7.2.1.1. Alarm Operation

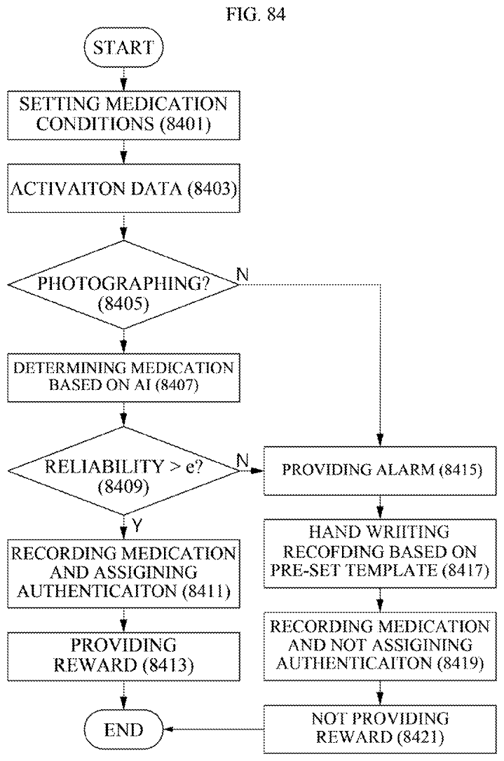

FIG. 84 is a flowchart illustrating an example of an operation for providing medication authentication and reward for a user of the health management system 1 according to various embodiments. Operations may be performed regardless of the order of the operations shown and/or described, and more operations may be performed and/or fewer operations may be performed.

According to various embodiments, the health management system 1 may set medication conditions in operation 8401, receive activation data in operation 8403, determine whether photographing is performed in operation 8405, determine medication based on an artificial intelligence model in operation 8407 when photographing is performed (8405-y), and determine whether the reliability of the medication determination result is greater than the threshold (e) in operation 8407.

According to various embodiments, the health management system 1 may assign medication records and medication authentication marks in operation 8411 when the reliability is greater than the threshold (c) (8409-Y), and provide reward in operation 8413. According to various embodiments, the health management system 1 may provide an alarm in operation 8415 when photographing is not performed (8405-N) or the reliability of the medication determination is less than the threshold (8409-N), record handwritten medication based on a template designated in operation 8417, perform medication records in operation 8419, and not assign authentication marks, and not provide reward in operation 8421. That is, the server 10 may not assign reward to the medication determination result in which the authentication mark is not assigned, thereby enhancing the medication management utilization using the health management system 1 by the user of the health management system 1. Meanwhile, the medication records may be performed based on a separate handwriting input of the user without photographing using the wearable device 20 and the medication authentication marks may be not assigned and reward may be not provided.

### 7.2.2. Reward According to the Judgment of the Medications by a Legitimate User

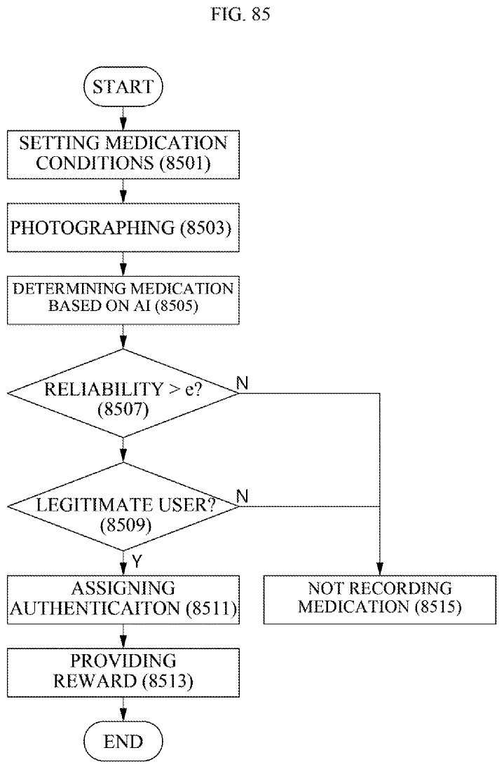

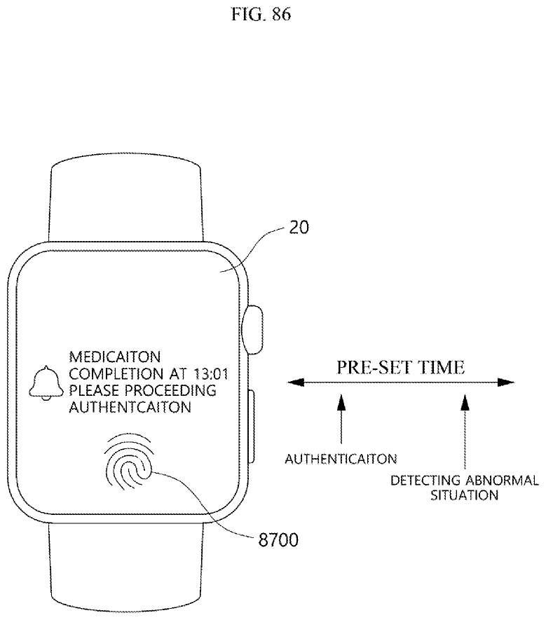

FIG. 85 is a flowchart illustrating an example of an operation for providing a reward based on the determination and rewarding of the medicine of the medical care in the health management system 1 according to various embodiments. Operations may be performed regardless of the order of the operations shown and/or described, and more operations may be performed and/or fewer operations may be performed. Hereinafter, FIG. 85 will be further described with reference to FIG. 86.

FIG. 86 is a diagram illustrating an example of a reward assignment and a determination of the medicine of the medical care in the health management system 1 according to various embodiments.

According to various embodiments, the health management system 1 may set a medicine condition in operation 8601, when photographing is performed in operation 8603, determine a medicine based on an artificial intelligence model in operation 8605, and determine whether the reliability of the medicine determination result is greater than the threshold (c) in operation 8607.

According to various embodiments, when the reliability is greater than the threshold (c), the health management system 1 may determine whether the medicine of the medical care is performed by the user in operation 8609. For example, when the medicine determination result is obtained, the wearable device 20 (or the user terminal 30) may determine whether a medicine is performed by the user of the medical care based on the obtaining of information for authentication of the user. For example, referring to FIG. 86, when the medicine determination result information is obtained, the wearable device 20 may request fingerprint information for user authentication to be obtained, and may identify whether the user of the medical care is by obtaining the fingerprint information. At this time, If authentication information is not received within a predetermined time or an abnormal gesture such as wearing after removal of the wearable device 20 is detected from the request time of the authentication information, the wearable device 20 may determine that the user is not the legitimate user.

According to various embodiments, when it is determined that the health management system 1 performs a medication by a medical user (8609-Y), the health management system 1 may grant authentication in operation 8611, and provide reward in operation 8613. In addition, according to various embodiments, the health management system 1 may not perform a medication record in operation 8615 when the reliability is less than or equal to the threshold value (c) (8607-N), or when it is not a medical user (8609-N). Meanwhile, the medication record may not be performed, but the medication authentication mark may not be granted as described above.

1. A device for medication determination, including:
a sub device including a first fastening structure configured to be detachable from a main body, the sub device being configured to be operated independently from the main body,
wherein the sub device includes:
at least one processor;
a short range communication circuit; and
a camera disposed to an external surface of the sub device;
wherein, while the sub device is coupled to the main body, the at least one processor is configured to:
establish, through the short range communication circuit, a communication connection with the main body,
capture an image related to a medication performed by a user using the camera, and
provide the image cause the main body to obtain a result information of the medication determination.

2. The device of claim 1, wherein the sub device further includes housing, and a flexible substrate disposed in the housing,
wherein the at least one processor, the short range communication circuit, and the camera is electrically connected through the flexible substrate.

3. The device of claim 2, wherein, when the sub device and the main device are combined and worn on the user's wrist, the camera is directed to photograph the user's hand.

4. The device of claim 1, wherein the at least one processor is configured to:
receive a request for a communication connection from the main device through the short-range communication circuit, and
establish a communication connection with the main device based on the received request.

5. The device of claim 4, wherein the at least one processor is configured to:
obtain identification information about a wireless communication device physically connected to a target object for medication from the main device through the short-range communication circuit, and
based on the identification information, obtain a signal containing activation data broadcast from the wireless communication device, set to activate the camera based on the activation data.

6. The device of claim 5, wherein the at least one processor is configured to:
establish a communication connection with the wireless communication device through the short range communication circuit, based on the identification information, and
obtain the signal including the activation data based on an established communication connection with the wireless communication device.

7. The device of claim 5, wherein the at least one processor is configured to:
through the short-range communication circuit, scan a signal containing activation data broadcast from the wireless communication device based on the identification information, and
based on the scan, obtain the signal including the activation data.

8. The device of claim 5, wherein the at least one processor is configured to:
transmit the captured image to the outside based on activation of the camera through the short range communication circuit,
wherein, the main device is configured to obtain result information of the medication determination, based on the transmitted image being analyzed by at least one artificial intelligence model of the server.

9. An operation method of device for medication determination including a main device and a sub device configured to be detachable from the main body, comprising:
establishing, through the short range communication circuit, a communication connection with the main body;
capturing an image related to a medication performed by a user using the camera, and
providing the image cause the main body to obtain a result information of the medication determination.

10. The operation method of claim 9, wherein the sub device further includes housing, and a flexible substrate disposed in the housing,
wherein the at least one processor, the short range communication circuit, and the camera is electrically connected through the flexible substrate.

11. The operation method of claim 10, wherein, when the sub device and the main device are combined and worn on the user's wrist, the camera is directed to photograph the user's hand.

12. The operation method of claim 9, further comprising:
receiving a request for a communication connection from the main device through the short-range communication circuit, and
establishing a communication connection with the main device based on the received request.

13. The operation method of claim 12, further comprising:
obtaining identification information about a wireless communication device physically connected to a target object for medication from the main device through the short-range communication circuit, and
based on the identification information, obtaining a signal containing activation data broadcast from the wireless communication device, set to activate the camera based on the activation data.

14. The operation method of claim 13, further comprising:
establishing a communication connection with the wireless communication device through the short range communication circuit, based on the identification information; and
obtaining the signal including the activation data based on an established communication connection with the wireless communication device.

15. The operation method of claim 13, further comprising:
through the short-range communication circuit, scanning a signal containing activation data broadcast from the wireless communication device based on the identification information, and
based on the scan, obtaining the signal including the activation data.
